(19) United States
(12) Patent Application Publication
(10) Pub. No.: US 2024/0029403 A1
(43) Pub. Date: Jan. 25, 2024

(54) AUTO HIGH CONTENT SCREENING USING ARTIFICIAL INTELLIGENCE FOR DRUG COMPOUND DEVELOPMENT

(71) Applicant: ViQi, Inc., Santa Barbara, CA (US)

(72) Inventors: Ilya Goldberg, Santa Barbara, CA (US); Christian A. Lang, Santa Barbara, CA (US); Dmitry Fedorov, Santa Barbara, CA (US); Kristian Kvilekval, Santa Barbara, CA (US); Katherine Yeung, Santa Barbara, CA (US); Henry Rupert Dodkins, Santa Barbara, CA (US); Teresa Findley, Santa Barbara, CA (US)

(21) Appl. No.: 18/215,158

(22) Filed: Jun. 27, 2023

Related U.S. Application Data
(60) Provisional application No. 63/355,667, filed on Jun. 27, 2022.

(51) Int. Cl. G06V 10/764; G06T 7/00; G06V 10/82; G06V 10/84; G06V 10/25

(52) U.S. Cl. CPC G06V 10/764; G06T 7/0012; G06V 10/82; G06V 10/84; G06V 10/25

(57) ABSTRACT

Methods and systems for machine learning are disclosed for automated high content screening of drug compounds. Functions in one method include, receiving an assay layout; receiving images of a plurality of wells in one or more plates; training binary AI models based on the positive phenotype controls versus negative control to generate probabilities of an input image being the positive control to which the binary AI models were trained; training an all-control AI model based on all of the positive phenotype controls and the negative control to generate a set of probabilities of an input image being one of the positive phenotype controls or the negative control; and generating one or more visual representations of the set of probabilities to evaluate performance of the trained all-control AI model and the binary AI models.

### CROSS-REFERENCE TO RELATED APPLICATIONS

This patent application claims the benefit of U.S. Provisional Patent Application No. 63/355,667 titled AUTO HIGH CONTENT SCREENING USING ARTIFICIAL INTELLIGENCE FOR DRUG COMPOUND DEVELOPMENT filed on Jun. 27, 2022, by inventors Ilya Goldberg et al., incorporated by reference for all intents and purposes. This patent application incorporates by reference U.S. patent application Ser. No. 17/650,067 titled MACHINE LEARNING FOR EARLY DETECTION OF CELLULAR MORPHOLOGICAL CHANGES filed on Feb. 4, 2022, by inventors Ilya Goldberg et al. for all intents and purposes. U.S. patent application No. 17/650,067 claims the benefit of U.S. Provisional Patent Application No. 63/228,093 titled MACHINE LEARNING FOR EARLY DETECTION OF CELLULAR MORPHOLOGICAL CHANGES filed on Jul. 31, 2021 by inventors Ilya Goldberg et al.; and also claims the benefit of U.S. Provisional Patent Application No. 63/146,541 titled MACHINE LEARNING FOR EARLY DETECTION OF CELLULAR MORPHOLOGICAL CHANGES filed on Feb. 5, 2021 by inventors Ilya Goldberg et al., both of which are incorporated herein by reference for all intents and purposes.

### FIELD

The disclosed embodiments relate generally to machine learning applied to high content screening of biological cells in drug assays for drug discovery and drug development.

### BACKGROUND

Various chemicals combined in compounds are often screened by drug or pharmaceutical companies to develop new drugs to treat various issues of biological cells found in various lifeforms, such as plants, animals, and human beings. The drug development process is a lengthy process with multiple steps or phases.

According to the United States (U.S.) Food and Drug Administration (FDA), the steps, stages, or phases are discovery and development, preclinical research, clinical research, FDA review, and FDA post-market safety monitoring. Each of these steps, stages, or phases can take years. In the drug discovery and development phase, thousands of drug compounds may be potential candidates for development of a medical treatment. Early testing is performed on the thousands of drug compounds to narrow down the number of drug compounds that look promising for further study.

Experiments can be run on drug compounds to gather information on: 1) How it is absorbed, distributed, metabolized, and excreted; 2) Its potential benefits and mechanisms of action; 3) The best dosage; 4) The best way to give the drug (such as by mouth or injection); 5) Side effects or adverse events that can often be referred to as toxicity; 6) How it affects different groups of people (such as by gender, race, or ethnicity) differently; 7) How it interacts with other drugs and treatments; and 8) Its effectiveness as compared with similar drugs.

Manually testing thousands of drug compounds takes considerable time and effort. Some drug compounds may be overlooked to reduce the number of drug compounds that undergo early testing to reduce the time and effort involved. Some testing or experiments of the drug compounds may be improperly overlooked or skipped during early testing in order to reduce the time and effort. It is desirable to reduce the time and manual effort in a different manner in order to get early results of drug compound effectiveness for a wide range of drug compounds.

Observing changes over an extended period of time in biological cells subject to a drug compound is difficult to do with the human eye, even when aided by a microscope. The changes to biological cells from a drug compound can be so subtle and can occur amidst a noisy environment such that the changes in the biological cells can be overlooked by a person studying hundreds of cells under a microscope. It is desirable to improve the capture and analysis of changes in numerous biological cells to drug compounds over periods of time.

### BRIEF SUMMARY

The embodiments are summarized by the claims that follow below and are incorporated herein by reference.

In some aspects, the techniques described herein relate to a method for drug discovery assays using one or more artificial intelligence (AI) models, the method including: receiving an assay layout defining one or more positive phenotype controls, at least one negative control, a plurality of drug compounds, a plurality of drug concentrations and their replicates in a plurality of wells of one or more plates, wherein the plurality of wells in the one or more plates to receive biological cells, drug compounds at specified concentrations, drug solvents, and/or carriers; receiving one or more images of each of the plurality of wells in the one or more plates, wherein each image includes a plurality of tiles or one or more sub-image regions; training one or more binary AI models based on the one or more positive phenotype controls versus negative control to generate probabilities of an input image being the positive control to which the one or more binary AI models were trained; training an all-control AI model based on all of the one or more positive phenotype controls and the at least one negative control to generate a set of probabilities of an input image being one of the one or more positive phenotype controls or the at least one negative control; and generating one or more visual representations of the set of probabilities to evaluate performance of the trained all-control AI model and the one or more binary AI models.

In some aspects, the techniques described herein relate to a system with one or more artificial intelligence (AI) models for drug design assays using machine learning, the system including: a first storage device storing one or more captured images captured at a subcellular resolution, each captured image capturing a plurality of biological cells treated with one or more known compounds over one or more concentrations; a computer system in communication with the first storage device, the computer system including a processor and a second storage device storing instructions for execution by the processor; a plurality of imaging artificial intelligence (AI) models stored in the second storage device for use by the processor, the plurality of imaging AI models including one or more imaging AI models to be trained to compare each concentration of each drug compound to target phenotypes of biological cells as defined by positive controls differentiating it from a negative control, one image AI model to be trained to distinguish all of the positive controls and the negative control from each other; one image AI model per drug compound to be trained to distinguish the concentrations of each drug compound to detect any concentration dependent phenotype for each drug compound independently of the target phenotypes, and one image AI model to compare all drug induced phenotypes to each other to detect phenotypic similarity between drug compounds; wherein the plurality of imaging AI models are used with instructions executed by the processor to process the one or more captured images stored in the first storage device to generate probabilities representing a mapping between cell observations of cells captured in the images and drug compound effectiveness for each trained AI model.

In some aspects, the techniques described herein relate to an apparatus including: an output device configured to display an AutoHCS report, the AutoHCS report including: for an all-control AI model over all controls, a similarity matrix with values illustrating similarities between all of the controls, a confusion matrix with values illustrating mistakes that the all-control AI model makes in classification over all samples, a per-image confusion matrix with values illustrating mistakes that the all-control AI model makes in classification aggregated per image, and a dendrogram illustrating the similarities between all of the controls; for a binary AI model per positive control of a set of one or more positive controls, a confusion matrix with values illustrating false positives, false negatives, true positives, and true negatives aggregated per image, a confusion matrix with values illustrating false positives, false negatives, true positives, and true negatives per sample, and a similarity matrix with values illustrating the similarities between the positive control and the negative control; per drug compound of one or more drug compounds, a violin plot of a self AI model illustrating any dose dependent phenotypes of the drug on the cells independently of a set of one or more positive controls and at least one negative control, one or more violin plots for each binary AI model illustrating similarity of the drug compound to each positive control, and a dendrogram for each drug compound concentration illustrating the similarity between each drug concentration and the set of positive and negative controls; and for an all-compound AI model, an all-drug compound dendrogram illustrating similarities of phenotypes in the cells induced by the drug compounds.

In some aspects, the techniques described herein relate to a system for drug discovery assays using one or more artificial intelligence (AI) models, the system including: a first plurality of input neural networks (1310) in an input layer (1301) to receive a plurality of pixels representing sub-image tiles of each well of a standard titer plate in a drug screening; a plurality of layers (1302) of a plurality of computational neural networks (1301) in communication with the first and second plurality of input neural networks (1310) of the input layer (1301) to receive the plurality of data inputs defining the assay layout and the plurality of images for each well of the standard title plate, the plurality of computational neural networks (1301) to analyze the plurality of sub-image tiles of each well for effectiveness of each different concentration of each different drug compound against a target phenotype of the biological cell exposed to each concentration of each different drug compound; a plurality of trained weights coupled to the plurality of computational neural networks to classify objects in the sub-image tiles and to detect the target phenotype of the biological cell in the plurality of sub-image tiles of each well; and a first plurality of outputs neural networks (1320) in an output layer (1303) in communication with a last layer (1315) of the plurality of layers (1302) of the plurality of computational neural networks (1301), the output layer (1303) receiving output data from the last layer (1315) of the plurality of computational neural networks (1301), the first plurality of output neural networks (1320) to generate probability data representing prediction of observable effect of each different concentration of each different drug compound causing the target phenotype of the biological cell being detected within the plurality of sample images for each well.

In some aspects, the techniques described herein relate to a flexible system for analyzing image-based assays, the flexible system including one or more artificial intelligence models (600) to provide the most accurate output results for a given monetary budget for analyzing images captured from the image based assays, the one or more artificial intelligence models trained on controls, drug compounds, and concentrations from an assay layout (602B) and a plurality of pixels representing sub-image tiles of the images (602A) captured from the image based assays, and once trained, the one or more artificial intelligence models (600) generate probabilities of output classes based on the images captured from the image based assays, the one or more artificial intelligence models (600) selected from the group consisting of a convolutional neural network (CNN) artificial intelligence (AI) image model (632), the convolutional neural network (CNN) artificial intelligence (AI) image model (632) including trained weights (1318) to generate the probabilities of the output classes based on the images captured from the image-based assays, and a feature-based image classifier AI model (634) to receive the plurality of pixels representing the sub-image tiles of each well of a standard titer plate in the drug screening, the feature-based image classifier AI model (634) to convert pixels into a feature vector that is then used by the feature-based image classifier AI model to classify objects in the sub-image tiles and generate the probabilities of the output classes based on the images captured from the image based assays; and an output report generator (638) in communication with one or more artificial intelligence models (600) to receive the probability outputs the from one or more artificial intelligence models (600), the output report generator (638) to generate a report (604) based on the images captured from the image-based assay.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1A is a block diagram of a client-server computer system with multiple client computers communicating with one or more computer servers in a server center (or the cloud) over a computer network, such as a wide area network of the internet, and the services offered.

FIG. 1B illustrates a block diagram of a biological cell analysis system implementing an early detection system for cellular phenotypical changes.

FIG. 2A illustrates a user interface window generated by the system including a slide viewer illustrating a brightfield image of biological cells (center) and annotations (right panel) of some of the cells.

FIG. 2B illustrates the detail of the right-side panel of the user interface shown in FIG. 2A.

FIG. 3 illustrates a user interface window generated by the system with an example output of machine learning classification of classified cells for detecting phenotypical changes in brightfield images.

FIG. 4A illustrates another user interface viewer window provided by the system.

FIG. 4B illustrates the viewer window of brightfield image of cells overlaid with the classification results from classification algorithms using a generated AI model.

FIG. 5A illustrates assay workflow diagrams, including a machine learning/model training phase with brightfield images of cells with known cell conditions and a diagnostic (inference) phase with cell classification of dosed cells to be tested for their cell condition by an AI model.

FIG. 5B illustrates a workflow diagram of the two phases, training, and classification for AI powered cell assays according to the disclosed embodiments.

FIG. 5C illustrates a workflow diagram and timeline in the generation of a report from images using one or more AI models in an AI platform.

FIG. 5D illustrates image capture of digital images of wells in a plate with biological cells and the formation of rectangular tiles of pixels from a digital image.

FIG. 6A illustrates a block diagram overview of an auto high content screening (AutoHCS or AHCS) system.

FIG. 6B illustrates components of an AutoHCS report generated by the auto high content screening system shown in FIG. 6A.

FIG. 6C illustrates a conceptual block diagram of the AI technologies that can be used for the ACHS system.

FIGS. 7A-7B illustrate examples of assay plate layouts part of a graphical user interfaces that is input into the AHCS system shown in FIG. 6A.

FIG. 7C illustrates a graphical user interface to receive an assay layout input from a user for which the input images have been captured.

FIG. 7D illustrates a conceptual diagram of an input file with an assay layout that can be read into the AHCS system shown in FIG. 6A.

FIG. 7E illustrates a conceptual diagram of a spreadsheet input associating sample photographs with the assay layout for N plates being analyzed.

FIG. 8A illustrates training of control AI models and a multi-class all-control AI model to generate output results.

FIGS. 8B-8D illustrates examples of the output results from the control models shown in FIG. 8A.

FIG. 9A illustrates training of per-compound concentration AI models and an all-compound concentration AI model.

FIG. 9B illustrates training of positive phenotype controls and binary AI models for generating results of a given drug compound for each phenotype being analyzed.

FIG. 9C illustrates training of an all-compound AI model for generating results to compare each of the drug compounds against each other and a negative control.

FIGS. 10A-10C illustrate example graphical output results to score drug compounds after the AI models are trained.

FIG. 11 (FIGS. 11A-1, 11A-2, 11B-1, 11B-2, 11B-3, 11C, 11D) illustrate an example report of graphical results including control AI performance scores for three positive controls; compound concentration scores for three different drug compounds; and raw score data to provide backup to the plurality of matrices, graphs, charts, and diagrams.

FIG. 12A illustrates a conceptual block diagram of a convolutional neural network system to provide the deep learning to analyze the tile images of the assay wells and generate the desired output results.

FIG. 12B illustrates a conceptual block diagram of a feature based AI system.

FIG. 13A illustrates a conceptual block diagram of an artificial neural network system to provide the deep learning to analyze the tile images of the assay wells and generate the desired output results.

FIG. 13B illustrates a block diagram of an artificial neuron for the neural network system shown in FIG. 12B.

FIG. 14 illustrates a block diagram of a client-server computer system with multiple client computers communicating with one or more computer servers in a server center (or the cloud) over a computer network, such as a wide area network of the internet.

FIG. 15 illustrates a block diagram of a computer system for use as a server computer and client computers (devices) in the system shown in FIG. 14.

### DETAILED DESCRIPTION

In the following detailed description of the disclosed embodiments, numerous specific details are set forth in order to provide a thorough understanding. However, it will be obvious to one skilled in the art that the disclosed embodiments may be practiced without these specific details. In other instances, well known methods, procedures, components, and subsystems have not been described in detail so as not to unnecessarily obscure aspects of the disclosed embodiments.

The disclosed embodiments include a method, apparatus and system for auto high content screening using artificial intelligence for drug compound development.

The initial phases of the drug discovery process can be broadly described as target discovery, lead identification, and lead optimization. Typically, lead identification requires screening through hundreds of thousands of candidate compounds using a selective, highly automated, and robust assay, which can be reliably performed millions of times.

These types of assays have traditionally been developed as a biochemical reaction in vitro that results in a simple, easily detectable readout like a color change, when a drug has the desired effect on its molecular target (usually a specific protein, or other cellular component). The initial stage of the discovery process was thus primarily concerned with identifying the molecular target, and establishing a cheap reliable assay for the desired effect.

In High Content Screening (HCS; or High Content Analysis, HCA or cellomics), the target for the screen is a cellular phenotype of clinical interest, not necessarily a pre-identified molecular target. This redirects the target discovery stage to identifying robust repeatable cellular phenotypes rather than molecular targets. This broadens the number of potential molecular targets for the desired clinical outcome, and allows assessing drug effects directly in vivo, but with the tradeoff that the assay is no longer reducible to a color change, and instead requires acquiring and processing images of cells with a microscope to identify the phenotype of interest.

That is, high content screening is a type of phenotypic screen conducted on cells that are exposed to various drug or chemical compounds. Whole cells or components of cells are analyzed. Hundreds or thousands of drug compounds are tested in parallel for their activity in one or more biological assays. The biological assays are looking for complex cellular phenotypes as output results. For example, phenotypic changes can include increases or decreases in the production of cellular products such as proteins and/or changes in the morphology (visual appearance) of the cell from the drug compounds. Hence HCA typically involves automated microscopy and image analysis

In high content screening, the biological cells are first dosed with the various concentrations of drug compounds being tested. After a period of time, the structures and molecular components of the cells are analyzed to see if the drug compounds were effective in the desired phenotypes expected. The cells can be marked or tagged with fluorescent markers. In other cases, florescent markers are not needed. Changes in cell phenotype can be measured using automated image analysis techniques. With different absorption and emission maxima for various fluorescent markers, a plurality can be conjugated with cells and different cell components can be measured in parallel. With high content images, changes at a subcellular level (e.g., cytoplasm, nucleus, and other organelles) can be analyzed. Therefore, a large number of data points can be collected per cell. In addition to fluorescent labeling, label free assays can be used. Brightfield images can be captured from the cells and analyzed for high content screening.

The disclosed embodiments can speed drug compound development using machine learning to develop a rapid automated HCS for identifying drug compounds with desired effects on biological cells. Machine learning can detect cell morphologies or phenotypes of biological cells induced by the drug compounds, even when these cell changes cannot be detected by human observers. The use of brightfield microscopy obviates cell fixation and staining which allows the technique to be more easily automated as well as making it faster and more cost effective. Besides brightfield images, other digital images of affected cells can be used, such as darkfield images from darkfield microscopy, phase contrast images from phase contrast microscopy, and differential interference contrast (DIC) images from DIC microscopy.

The principal advantage of using machine learning for these types of screens is that the target phenotypes are used to train the AI by example rather than the traditional technique of describing the target phenotypes using prior image processing algorithms. Another advantage of using AIs is that they can recognize phenotypic differences that humans cannot observe directly. It is not necessary for a human to confirm that a positive control has exhibited a phenotype by direct observation.

In other disclosed embodiments, cloud-based image processing and machine learning can bring automated high content screening (HCS) to the drug development process to increase the pace of development of novel drug compounds. The ability to detect changes in single cells and differentiate from other types of cellular changes can dramatically alter the time and effort currently required for HCS of drug compounds.

HCS has proven invaluable to early drug discovery programs. The disclosed embodiments can improve on traditional HCS by leveraging automation and artificial intelligence to produce rapid turn-around and screening of potential drub compounds.

The disclosed embodiments provide automated high-content screening (HCS) platforms and thus more easily fit into existing drug discovery pipelines. Given the significant cost of drug development, additional subscription fees for a machine learning platform are negligible.

The disclosed embodiments use artificial intelligence (machine learning) to recognize patterns without the need for human intervention. The broad principals disclosed can be applied to HCS and HCA assays as detailed below. Equally, the disclosed embodiments are also applicable to drug dosage response of cells, cell toxicity, and detection of live/dead cell detection with or without the use of fluorescence labelling.

The disclosed embodiments use machine learning to accelerate identification of desired phenotypes by various drug compounds. The disclosed embodiments take a novel approach to HCS by training modern artificial intelligence (AI) models to identify compounds with a desired phenotypical effect on biological cells, and score and report those morphological effects.

The AI models disclosed identify cells based on morphological or phenological changes in the cells. The AI models process images of cells in biological samples and can also be referred to herein as imaging artificial intelligence (AI) models. The use of AI obviates the need for several slow elements of the assay protocol, resulting in increased throughput for evaluating potential drugs. AI can facilitate automation of high-content screening (HCS) platforms and thus more easily fit into existing drug discovery pipelines.

Modern artificial intelligence models (AIs) have not been previously used to identify and score affected cells. The disclosed embodiments can use stained or unstained cellular images with AI models to discern differences in cells and tissues that cannot be differentiated by highly trained human observers. Artificial intelligence models (AIs) can readily differentiate sub-cellular patterns that even trained human experts could not with immuno-fluorescence assays on individual cells. Human observers cannot do so without labels or stains. Moreover, human observers cannot quantify the similarities between phenotypes of a large quantity of compounds and concentrations and positive control phenotypes. The disclosed embodiments can provide crucial research tools to drug development experts to more quickly develop drugs.

Generally, the disclosed embodiments perform and automated HCS by training AI models to recognize cells with similar morphologies to control cells, score the similarities, and report the results, without requiring a significant amount of capital investment for custom software development or pattern recognition algorithms, microscopy image format readers, scalable slide viewers, data processing for machine learning, model evaluation and sharing. Some of the disclosed embodiments include a web-based interface to minimize time-consuming installation and configuration of custom software and hardware.

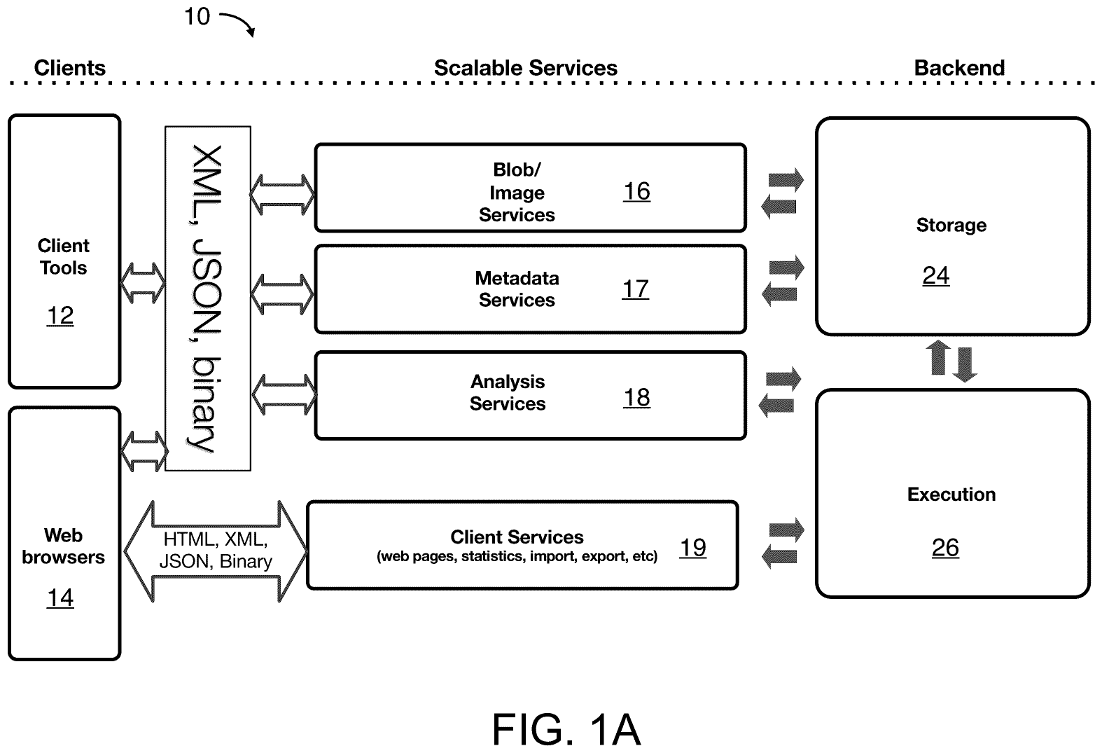

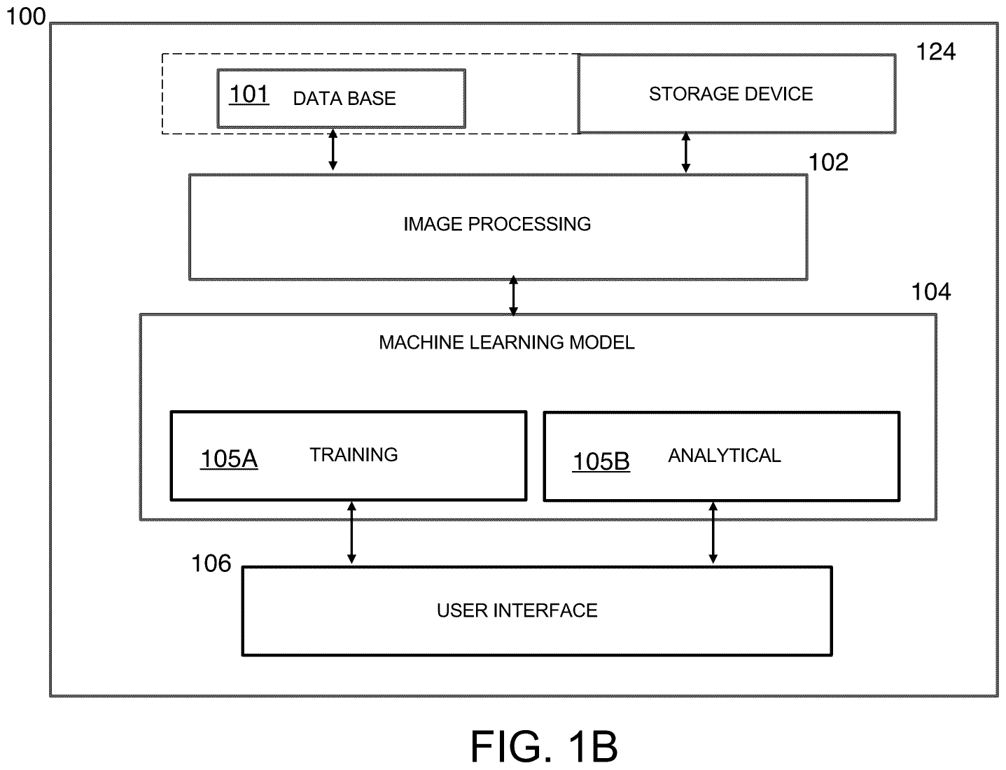

Referring now to FIG. 1A, a web-based scalable image analysis platform 10 is shown. The web-based scalable image analysis platform 10 is usable for a variety of image management and analysis tasks in life sciences. A vast majority of images processed by the web-based scalable image analysis platform are high resolution microscopy images.

The web-based scalable image analysis platform 10, modeled after a BisQue system, can be used to analyze biological cells. BisQue is an open-source image database and analysis system built for biologists working with digital artifacts as a fundamental tool for gathering evidence. The system is built to manage and analyze both data artifacts and metadata for large-scale datasets. BisQue has gained increasing adoption by several large bio-imaging labs world-wide. For example, the CyVerse cyber-infrastructure serves thousands of users using BisQue for its imaging needs.

The web-based scalable image analysis platform 10, being a cloud-based platform, is built with a scalable service-oriented architecture. The web-based scalable image analysis platform 10 provides access to services through web browsers 14 and client tools 12. The web browsers 14 access client services 19 including web pages with viewers and statistics and can be used for general control, such as file import and file export.

Web browsers 14 and client tools 12 can interface to a plurality of micro-services 16-18, each performing a specialized task. A web browser can also act as a client tool (JavaScript) and interface to the microservices 16-18 (e.g., running analysis modules from a webpage). A metadata service 17 allows metadata to be stored in storage 24 and queried as nested tag/value documents. Examples for metadata include sample preparation, experimental conditions, imaging parameters, and results. A blob and image service 16 provides access to binary datasets such as images and provides methods to perform pixel operations for example. An analysis service 18 allows running complex analysis modules written in many languages (e.g., Python, MATLAB). These analysis modules of the analysis service 18 typically interact with the other services (e.g., to fetch image tiles or metadata records) during computation. The microservices 16-18 and the client services 19 can interface to the backend services of storage services 24 associated with storage devices and execution services 26 associated with computer processors or compute clusters.

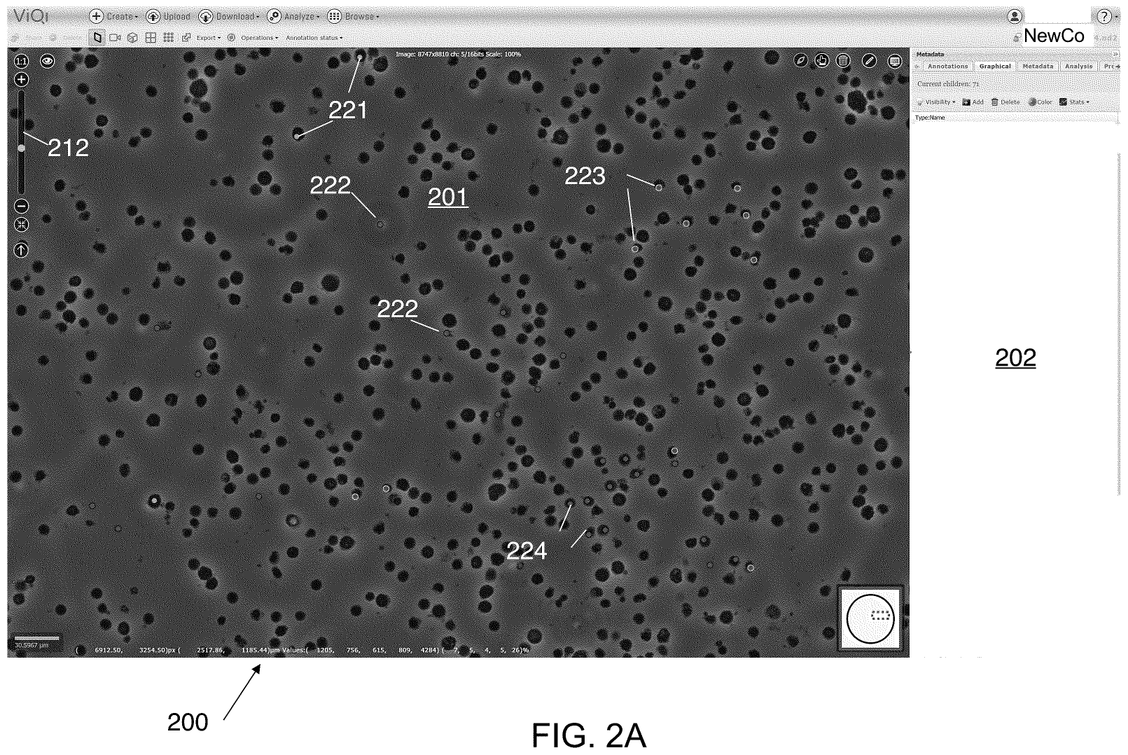

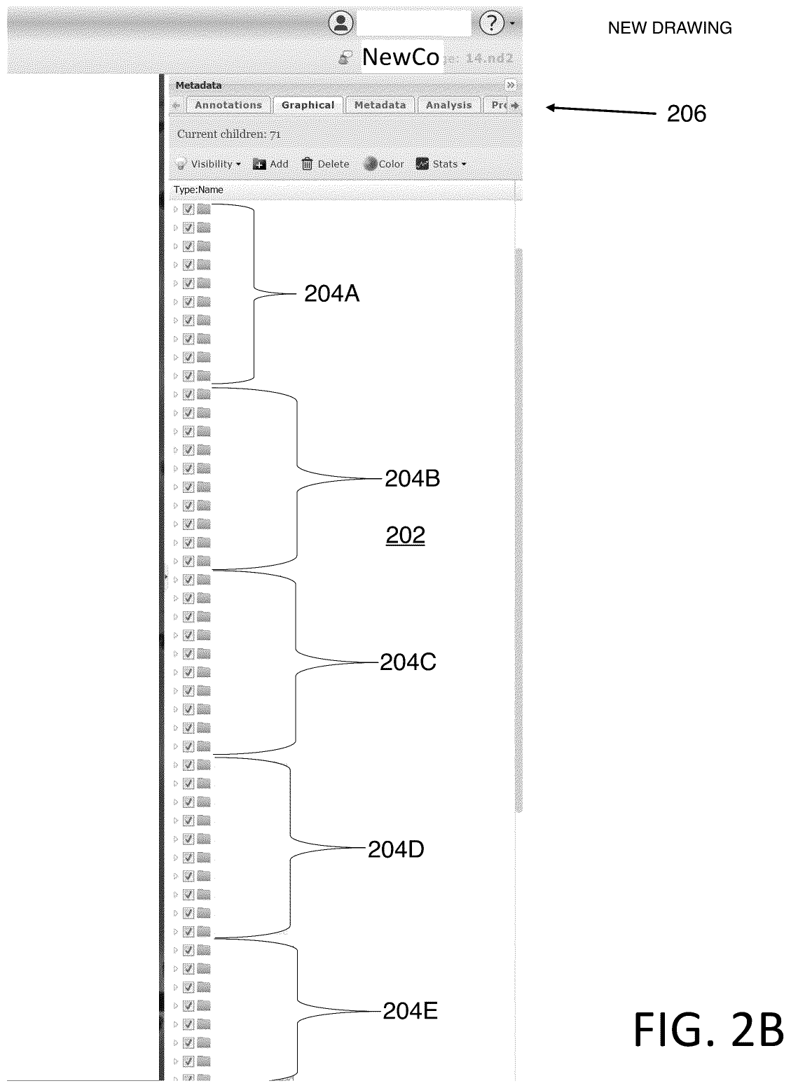

The platform supports all popular microscopy image formats (including channels and time series). The platform has a slide viewer that allows visualization and annotation of images of any size, with multiple channels and of any bit depth. FIG. 2A shows a user interface 200 with an example high-resolution image of a portion of a biological sample in a viewer window 201. The example high-resolution image is a five (5) channel, sixteen (16) data bit-width image taken from a high resolution digital microscope or other high resolution digital imaging device (digital imager). In one embodiment, the digital imaging device is an imaging robot that performs robotic microscopy. Additional robots, such as a fluid handling robot, can be used to process the plates for imaging in order to provide a more automated process.

A major criteria of image acquisition and analysis is the primary acquisition and timely transfer of images for cloud processing. The high resolution images tend to be large and difficult to transfer by email. The majority of users can utilize internet transfer mechanisms to upload images either directly from the acquisition device or from local network-attached-storage (NAS).

Optimally raw image data, experimental metadata including cell-line, and per well data such as compound concentration and replicates are needed for an accurate analysis. Ideally, this functionality would be integrated into the acquisition pipeline through close integration into the software controlling the imaging, but these devices may have incompatible or prohibitive security, operational or hardware requirements.

For instances where there can be incompatibilities or other issues with integrated acquisition of data, custom software can be used to interrogate the client software for the needed data. Toolkits and services can be deployed locally to automatically and securely transfer images and metadata as acquired. In some cases, if direct access to the acquisition machine and its raw image data is not possible, raw image data can be manually or automatically imported or exported. A toolkit can be structured so that collection of raw image data can be imported with multiple strategies: from application database, from filesystem export, etc. Experimental meta-data is usually not reported by imaging devices unless they are part of a LIMS (laboratory information management system). The meta data is acquired with the raw image data.

Experimental layouts are represented on a grid using a spreadsheet. Spreadsheet templates for experimental layouts can be provided to clients to facilitate the uniform importation of data. Parsers and web user interface (UI) components can also be used on user-provided spreadsheets. Alternatively, images and metadata can be transferred directly through the web browser via a proprietary web portal. In the event of limited or no connectivity due to poor infrastructure, regulatory, or security requirements, local storage and processing can be made available for large-scale users.

Referring now to FIG. 1B, a high-level block diagram of a biological cell analysis system 100 is shown that utilizes machine learning to detect early-stage cellular morphological changes. The biological cell analysis system 100 includes an image processing system 102, a machine learning (AI) model 104 that can be either trained or used for classification/analysis, and a user interface 106. The machine learning (AI) model 104 can be trained for use with one or more machine learning algorithms (including image processing algorithms) and then used with the one or more machine learning algorithms for classification/analysis of objects within images of biological samples (e.g., treated or untreated), including biological cells and their sub-cellular structure.

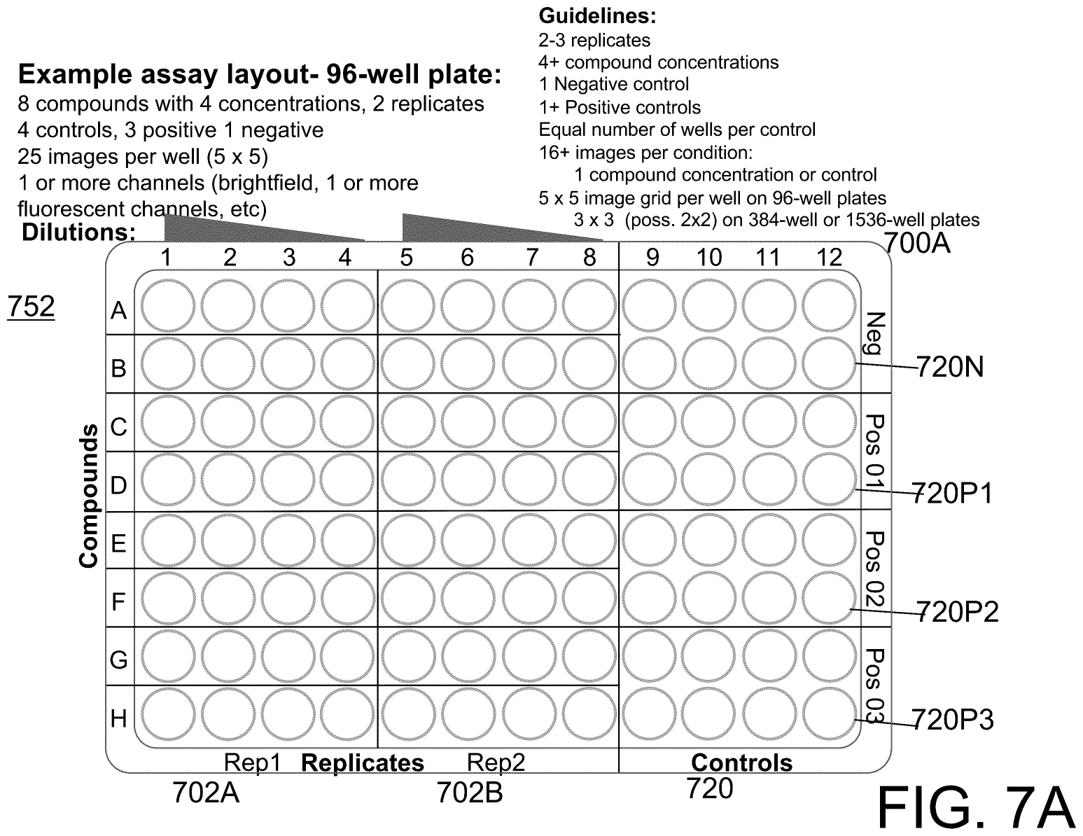

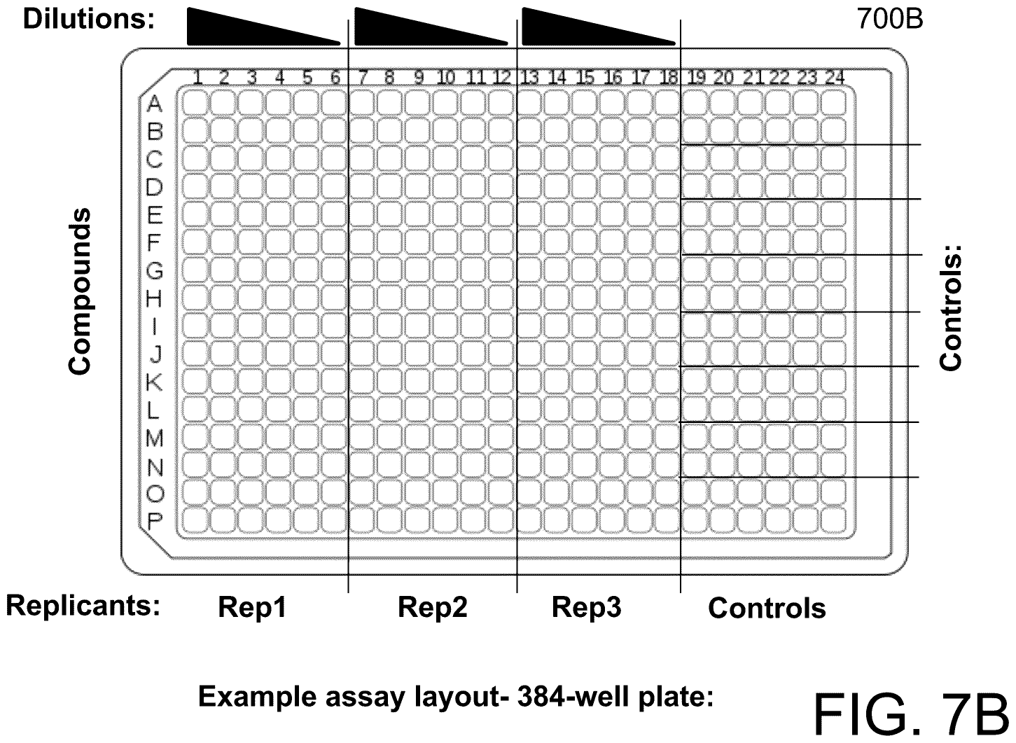

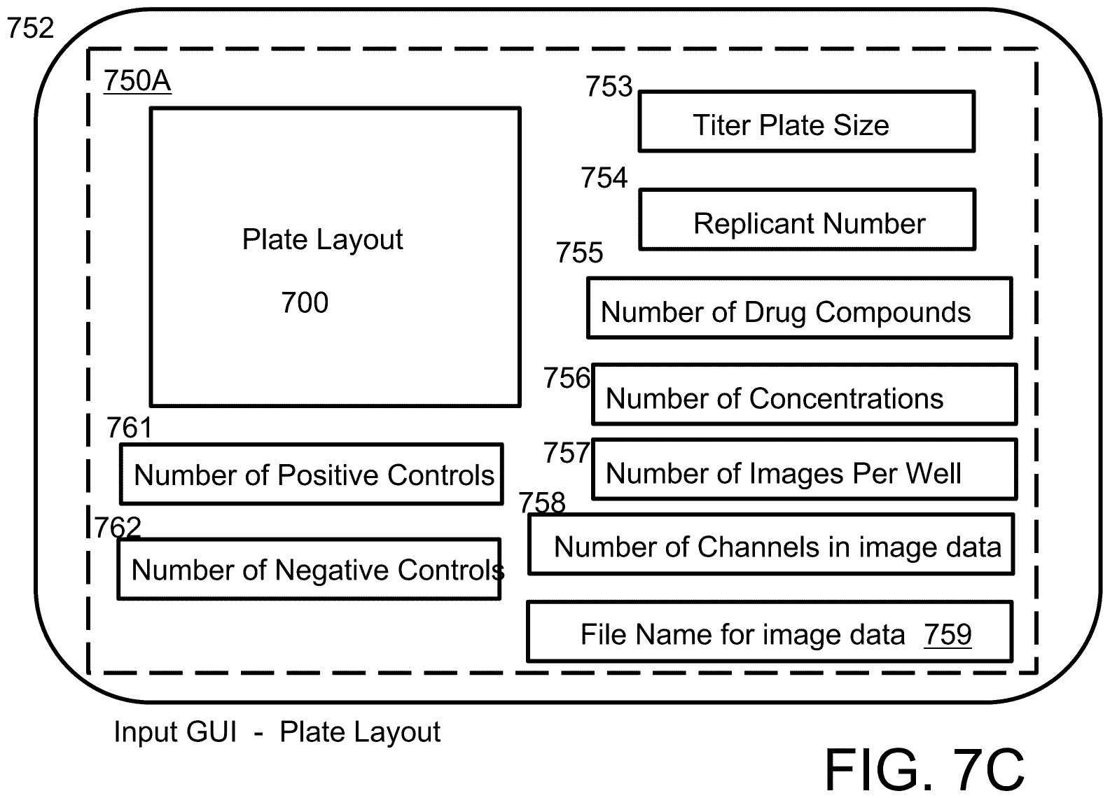

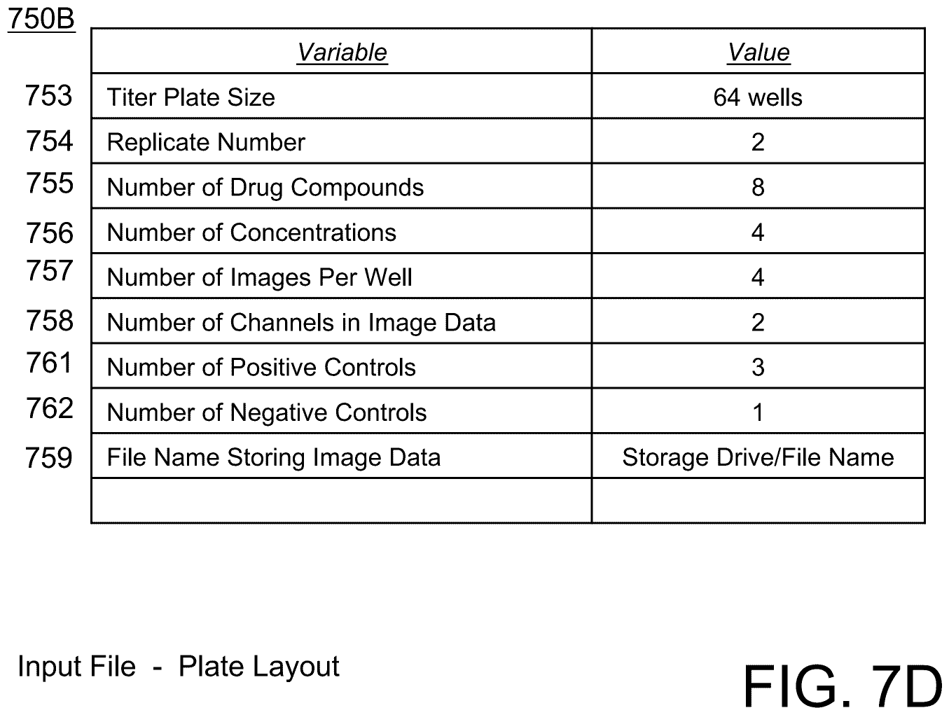

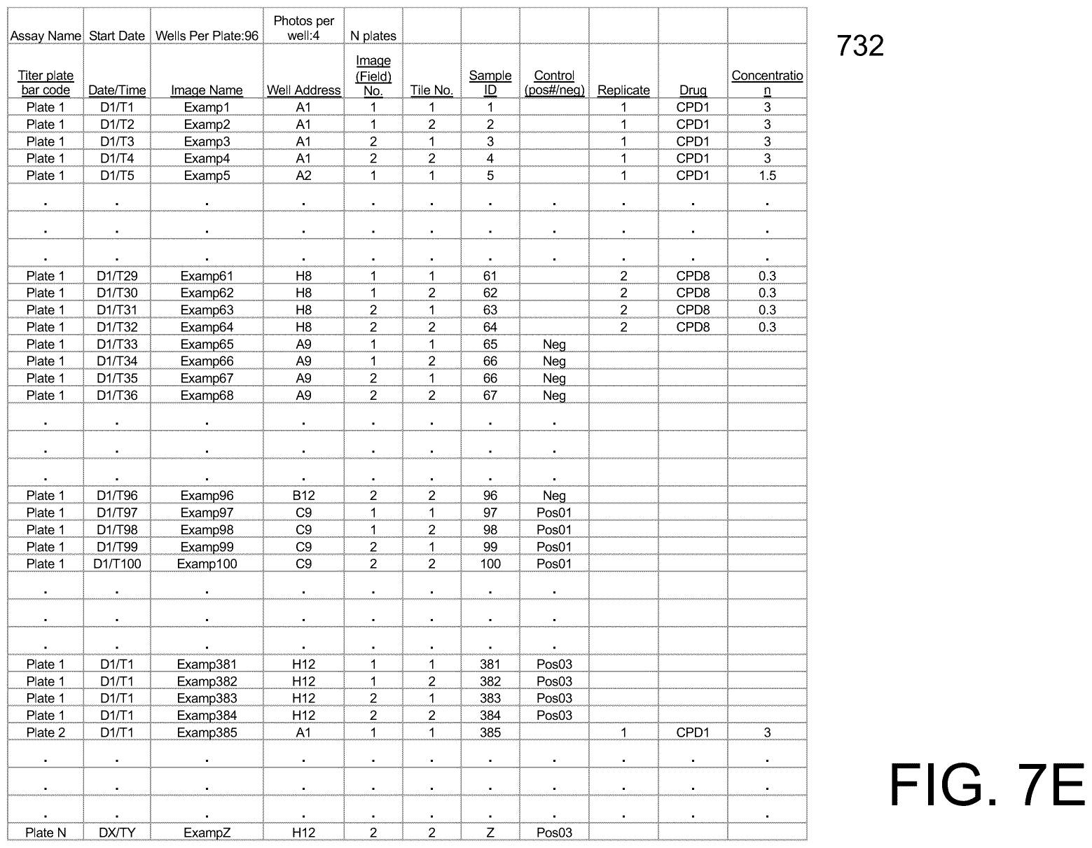

The image processing system 102 can read digital images of cells stored in a storage device 124 and associated metadata from a database 101. The raw image data and associated metadata can be respectively transferred from a user's storage device/database via the internet into the storage device 124 and the database 101 for cloud image processing. In one embodiment the database 101 is stored in the storage device 124 with the images while in another embodiment, the database 101 is stored in another storage device (e.g., memory 720, SSD 730, Disk Drive 740 shown in FIG. 7) separate from the storage device 124 storing the raw image data. In other cases, the database 101 and the storage device 124 can be local and local processing by one or more local processors with local software can be utilized for clients having sufficient hardware and volume of stored assays.

Once the image data and experimental metadata are available in the database 101, the image processing system 102 can be used to process raw image data. The raw image data can be processed to obtain specific image information (e.g., resolution, size, background filtering, etc.) The image processing system 102 can generate feature vectors from the captured images of the biological cells. The feature vectors can be used to train the machine learning (AI) model 104.

The machine learning (AI) model 104 can be trained to provide useful and accurate information about the biological cells and other objects within a biological sample. In one embodiment, the machine learning (AI) model 104 is used with one or more classifier algorithms to classify biological cells and to detect morphological changes of the classified biological cells over time. The biological cells can be dosed with one or more drug compounds and different concentrations with targeted phenotypes in the biological cells with images captured over one or more time periods. One or more AI models 104 can be used to generate a set of probabilities associated with the targeted phenotypes from the captured images. One or more visual representations can be generated from the set of probabilities and displayed to a user.

Once the image data is processed by the image processing system 102, the image data can be used to train an AI model 104 in a training mode 105A. The AI model 104 can be validated with additional image data that is excluded from the training process. If the AI model 104 has been previously trained (pretrained), it can be used in an analytical mode 105B to screen a plurality of drug compounds for one or more effects of interest observable as morphological or phenotypical changes to the cell(s). In the analytical mode 105B, images of biological cell assays can be screened to recognize biological cells based on target phenotypes. The phenotypical effects of the plurality of drug compounds on the imaged cells at differing concentrations are compared to the target phenotypes exhibited by positive controls. Scores are generated quantifying the similarity of the drug compound effect to the target phenotype.

A user can interact with the system 100 through the user interface 106. The user interface 106 can be used to build one or more AI models for a new sample of biological cells. The user interface 106 can be used to seed the recognition/classification of one or more objects in the new sample. The user interface 106 can receive the report generated by the use of the AI model 104 and algorithms in analyzing the one or more images of a biological sample. The report and analytical results can be viewed in various windows generated by the user interface 106 on a display device.

FIG. 2A illustrates a user interface 200 with a display window 201 displaying a brightfield image of biological cells in the brightfield channel. A number of objects 221, 222, 223, and 224 that are associated with the biological cells shown in FIG. 2A. The objects can be classified with different tags or labels. FIG. 2B illustrates the right panel 202 of annotations of the different tags or labels for the objects 221, 222, 223, and 224 associated with the biological cells. Different colored circles on a few objects recognized by a user within the image are tagged and labeled as shown in FIG. 2A-2B during training. Thereafter, similar objects can be recognized/classified by machine learning, tagged or labeled in the image with annotations (type of object, location) added as part of the metadata 206 of an image. The tagged or labeled objects in the image can be colored differently, such as by different colored circles, and can be overlaid onto the objects (e.g., cells, debris) such as shown in the image of FIG. 2A. The different colors can be used to emphasize the annotation and provide information about the tagged object, such as a cell or a subcellular component (e.g., nucleus, lysosomes, peroxisomes, mitochondria, endoplasmic reticulum, golgi apparatus) and its state (live, dead, treated, untreated, etc.).

As shown in FIG. 2A, the annotations associated with the objects 221-224 with tags or labels can include Debris 204A in a first color, CellX_live 204B in a second color, CellX_dead 204C in a third color, CellY_live 204D in a fourth color, and CellY_dead 204E in a fifth color, for example. The annotations shown in FIG. 2B can be colored to match that of the colored circles overlaid onto the objects (e.g., cells, debris) shown in the image of FIG. 2A. The tags or labels on the objects 221-224 and associated annotations, are used to train one or more AI models of supervised machine learning algorithms so that the same classes of objects can be recognized throughout the image at different locations.

In FIG. 2A, a slider 212 can be selected by a user input device and adjusted up or down to magnify or demagnify the selected portion of the brightfield image of the sample displayed in the viewer window 201 of the user interface 200. An associated up button can be pressed by the user input device to zoom in on the sample portion displayed in the viewer window 201. An associated down button can be pressed by a user input device to zoom out on the sample portion displayed in the viewer window 201.

### Machine Learning Service

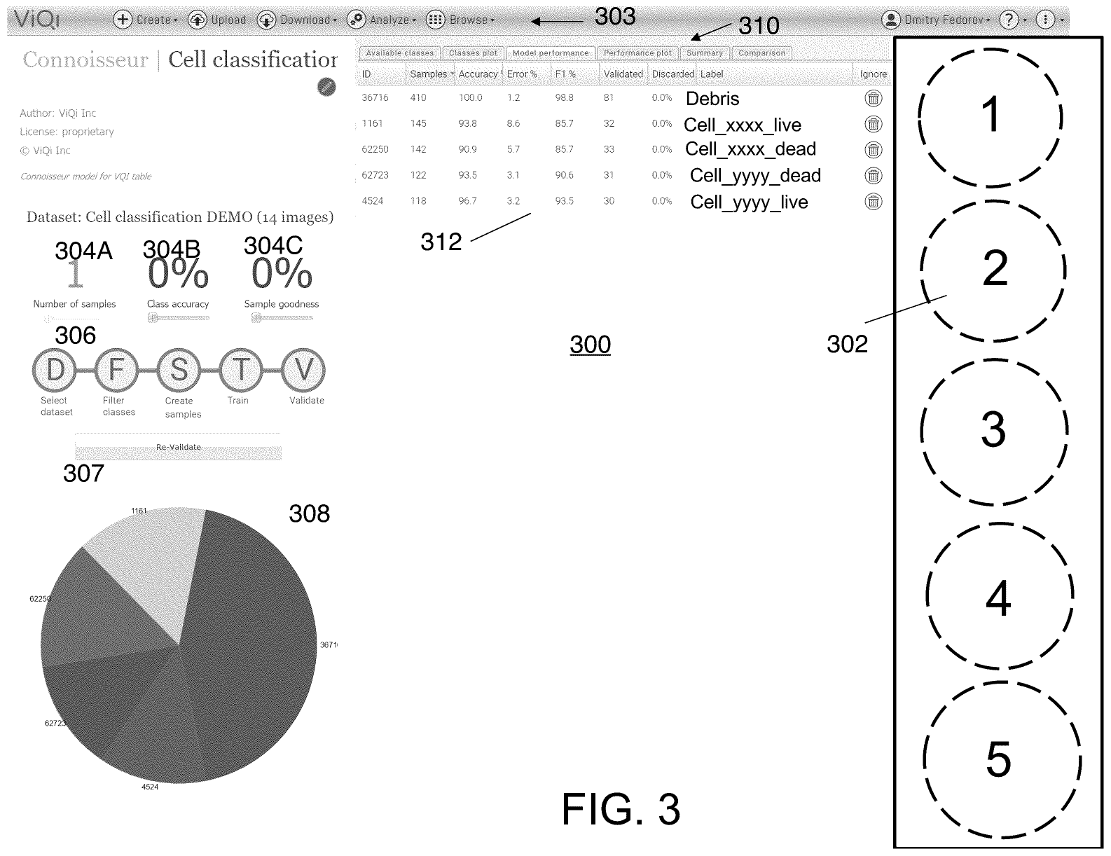

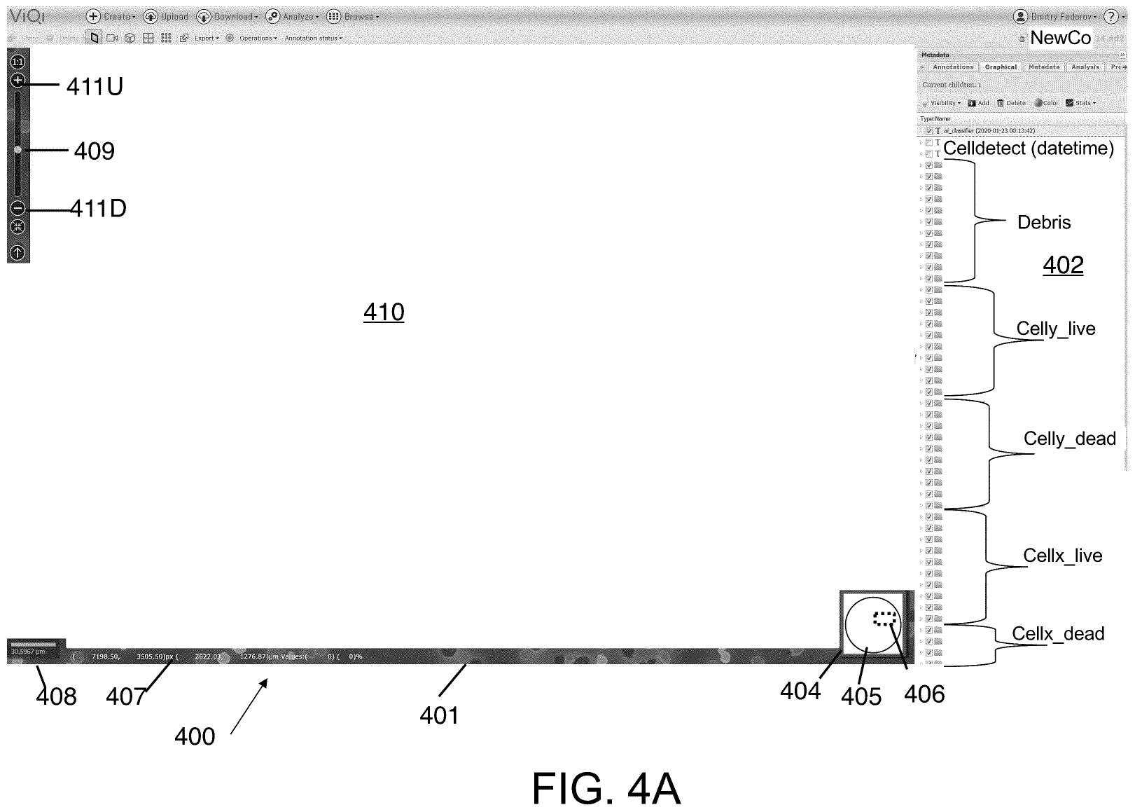

Referring now to FIG. 3, the web-based scalable image analysis platform 10 shown in FIG. 1A, includes a machine learning service as part of the Analysis Services 18 to build AI models and classify cells and other objects. FIG. 3 illustrates a deep learning model builder 300 as part of the platform. An example imaging AI cell classification model shown in FIG. 3 is used for classifying live/dead cells in a sample of cells. The model builder 300 illustrates a sample 302 (sample two of one through five) of a training dataset that is shown in the right side of the frame. A row of control buttons 303 at the top of each page, including Create, Upload, Download, Analyze, and Browse, can be used for creation, uploading, downloading, analyzing, and browsing.

A slider and display 304A can be used to filter out classes based on minimum number of samples per class. A slider and display 304B can be used to select a minimum accuracy required for a class to be used in classification. A slider and display 304C can be used to select a goodness threshold under which classification results for individual samples will be discarded during classification. A progress bar 306 of the steps in building the AI model is provided, including selecting the dataset, selecting the filter classes, creating samples, training of AI model, and validating the AI model. The user interface guides the user through each of these steps of the process, shown by the progress bar 306. If something changes in one or more of the settings, a revalidate button 307 is provided to re-validate a previously trained and validated AI model. The builder includes a pie chart 308 that visibly shows, with different colors, the size of the different object classes found in the training dataset.

The builder 300 further shows a plurality of tabs 310 that can be selected to show information about the AI builder including available classes of object that can be recognized, a plot of the classes of objects used during model building, model performance table (shown), a performance plot of objects, a summary table, and a comparison table. The selected tab (model performance) shows a model performance table 312 of the object classes recognized by the AI model for the selected training dataset. It is information used during training of the AI model. For example, in a row of the table 312 for the class of Cell_xxxx_live cells having ID 1161, the AI model and classifier included 145 cells from the training dataset, resulting in a 93.8 percent accuracy, an 8.6 percent error rate, an 85.7 percent F1% rate (a metric quantifying the prediction accuracy), with 32 cells from the training dataset used for validation, and 0.0 percent of the cells discarded during training.

The machine learning service allows a user to build machine learning models on large-scale training data collected from images and metadata with a variety of features and learning frameworks. The generated models can be used to predict properties over large image collections and visualize the results as overlays for easy digestion by scientific users.

Some phenotypic events are not visible to human observers. Membrane-bound structures associated with viral infection can be seen by electron microscopy and they are of sufficient size to have a readout in visible light, but they have not been observed manually with light microscopes. In contrast, AI models have been trained to observe many phenotypic effects in cells using brightfield microscopy that are invisible to human observers. It has been demonstrated that AI models can be trained to detect subtle morphological changes in treated cells that are invisible or otherwise unremarked by human observers viewing the same or similar images.

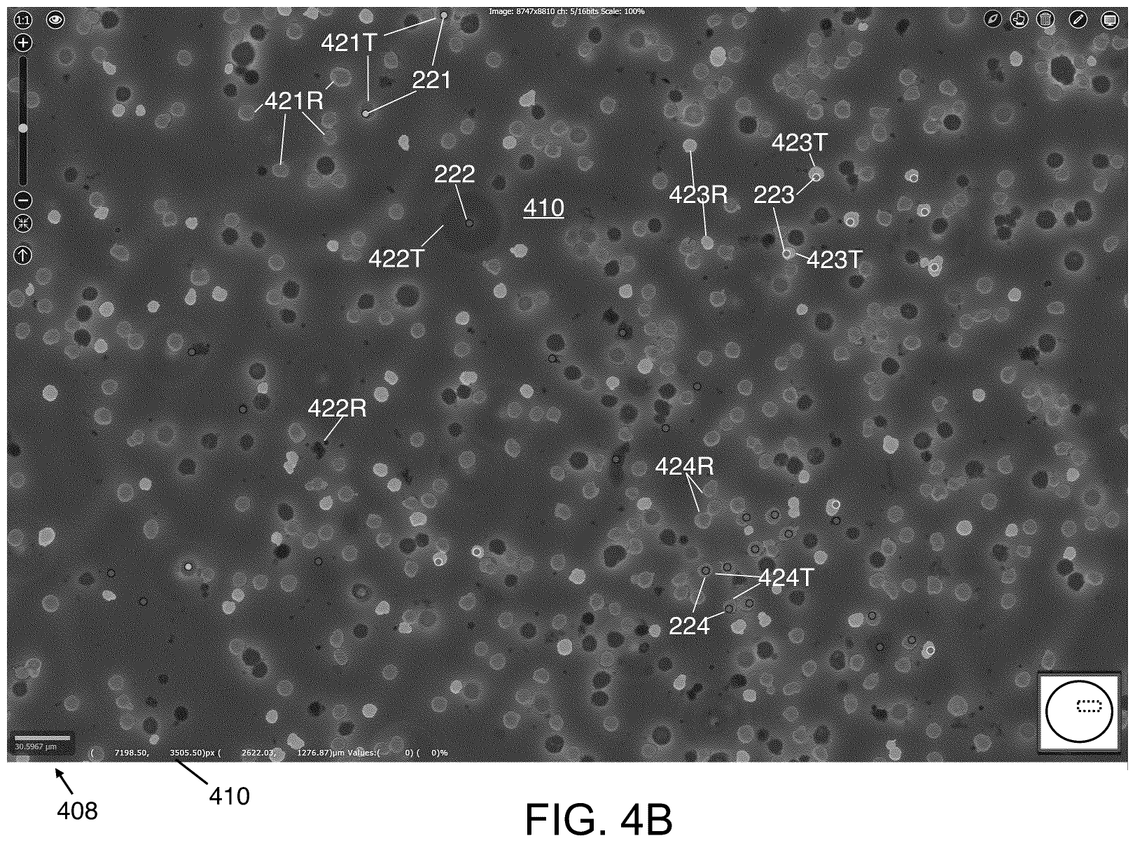

FIGS. 4A-4B show a user interface window 400 with an example resultant output of a machine learning classification with classified (e.g., live/dead) cells and other objects recognized in brightfield images and color coded using an object outline overlay. In FIG. 2A, tags or labels 221-224 were added on top of objects and associated annotations were created. In FIG. 4B, these tags and labels 221-224 are used to train one or more AI models of supervised machine learning algorithms to train on the training objects 421T,422T,423T,424T. With these trained AI models, the supervised machine learning algorithms can recognize the same classes of objects as recognized objects 421R,422R,423R,424R throughout the image at different locations during analysis after training. Besides tags or labels added by a user, AI models can be trained using fluorescent tags (fluorescent protein genes, fluorescently labeled antibodies, etc.) to mark cells of interest.

Multi-channel imaging with a fluorescence channel and a brightfield channel can be used to train AI models. One or more fluorescent probes can be used to mark specific phenotypes, like nuclear entry of a specific receptor, or fluorescent probes can be used to highlight the gross morphology of cellular structures and subcellular organelles. Likewise, brightfield channel can be used alone or with fluorescence channels to probe changes in gross morphology of the cells.

In FIGS. 4A-4B, the viewer overlays the classification results on top of the image as color-coded masked regions. For example, the color red can be overlaid onto certain cells to indicate the dead cells to the user. The color yellow, for example, can be overlaid onto certain cells to indicate live cells. Other colors can be used instead and other cell states can be indicated by more colors as well. The visualizer (platform slide viewer) allows for smooth navigation of large images, even with millions of such regions.

As shown in FIG. 4A, the user interface window 400 includes a viewer window 401 and a side bar 402. In the viewer window 401, a magnified portion 410 of the sample is shown. The viewer window 401 includes an overview window 404 of the sample in a well 405. The magnified portion 410 of the sample shown in the viewer window 401, is indicated in the overview 404 by a rectangle 406 in the well 405. Magnification information 407 is overlaid onto the portion 410 in the viewer window 401. A scale 408 is overlaid onto the portion 410 displayed in the viewer window 401. Magnification adjustment controls 409,411U,411D are overlaid on the portion 410 to allow adjustment of the portion 410 of the sample displayed in the viewer window 401. A slider 409 can be selected by a user input device and adjusted up or down to magnify or demagnify the portion 410 of the sample displayed. An up button 411U can be pressed by the user input device to zoom in on the sample portion displayed in the viewer window 401. A down button 411D can be pressed by a user input device to zoom out on the sample portion displayed in the viewer window 401.

After a screen is run with the AI model on one or more images of cells, the side bar 402 can indicate the various objects (e.g., treated cells, untreated cells, subcells) recognized and displayed in the viewer window 401. The side bar can also indicate the AI model that is used to generate the objects and the date and time the analysis is run with the AI model. The side bar can also indicate the user provided metadata that was used to train the AI model.A

The magnified portion 410 of the sample shown by the viewer 401 is illustrated in FIG. 4B. Over that of the viewer window of FIG. 2A, objects are detected, classified and shown by the different colors after an analysis.

In addition to the above-mentioned features, the web based platform has built-in support for sharing and collaborating among scientific teams. Because it is substantially web-based, no complex software installations are required to get started analyzing biological cells. The capabilities of the web-based scalable image platform 10 make it easy to use the system for the early detection of changes in cellular structure.

The disclosed embodiments train imaging artificial intelligence models (AIs) to classify phenotypic effects on individual, labelled or unlabeled cells treated with a drug compound. This allows screening to be conducted in a highly automated and cost effective manner, potentially shortening the drug discovery process while saving the user a substantial sum of money.

In addition to the innovative machine learning aspect, the developed models are shareable worldwide using a web-based platform. This is innovative as model sharing in the past meant setting up complex software systems to read such models and then performing classifications based on them. By using a web-based platform, sharing of models merely requires a web-browser. Efficient sharing of models among scientific teams is crucial for the rapid development of urgently needed drug treatments.

### Training and Classification Workflow

One main objective of the embodiments is to reduce the time to obtain screening results of drug compounds by the use of machine learning.

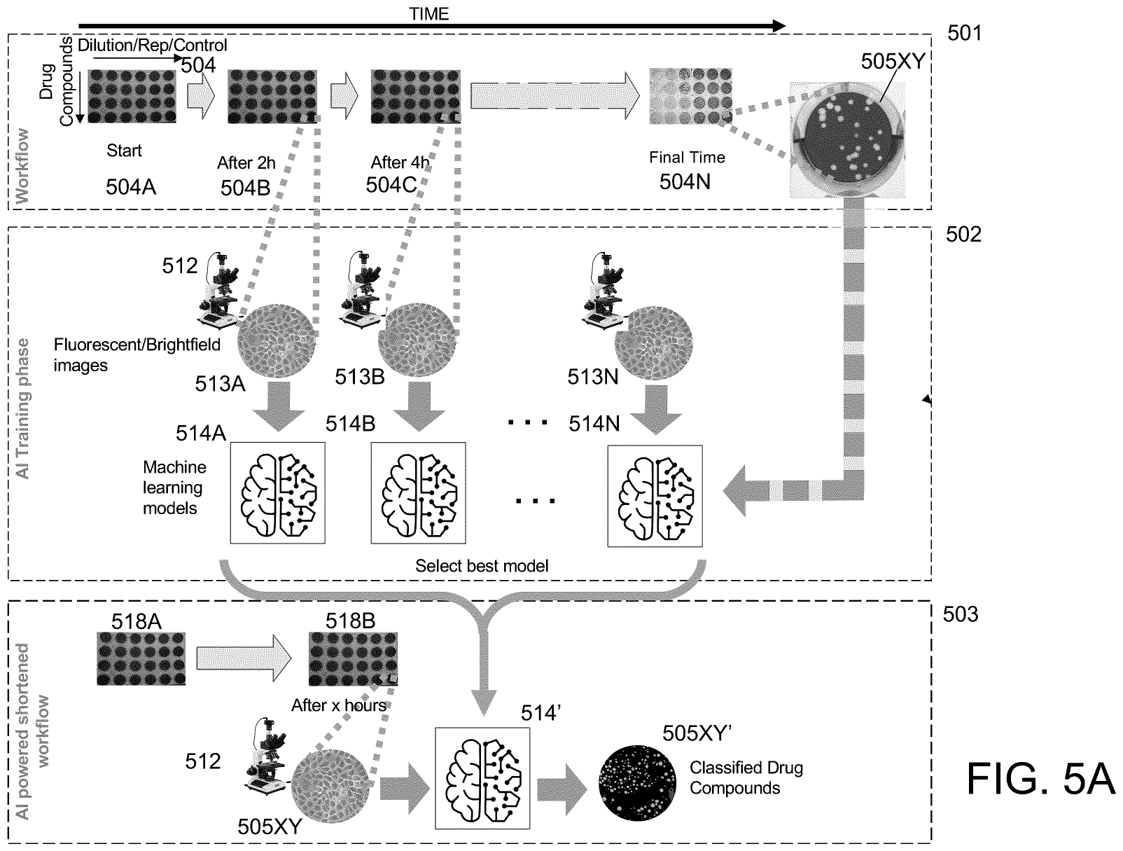

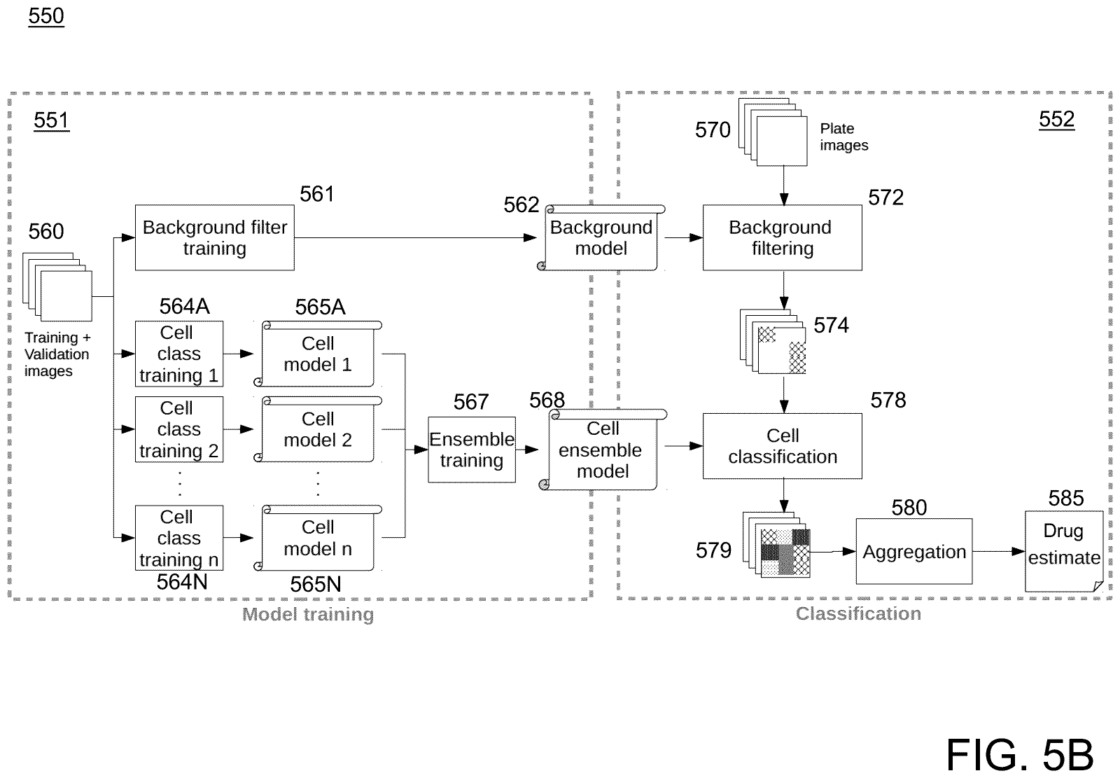

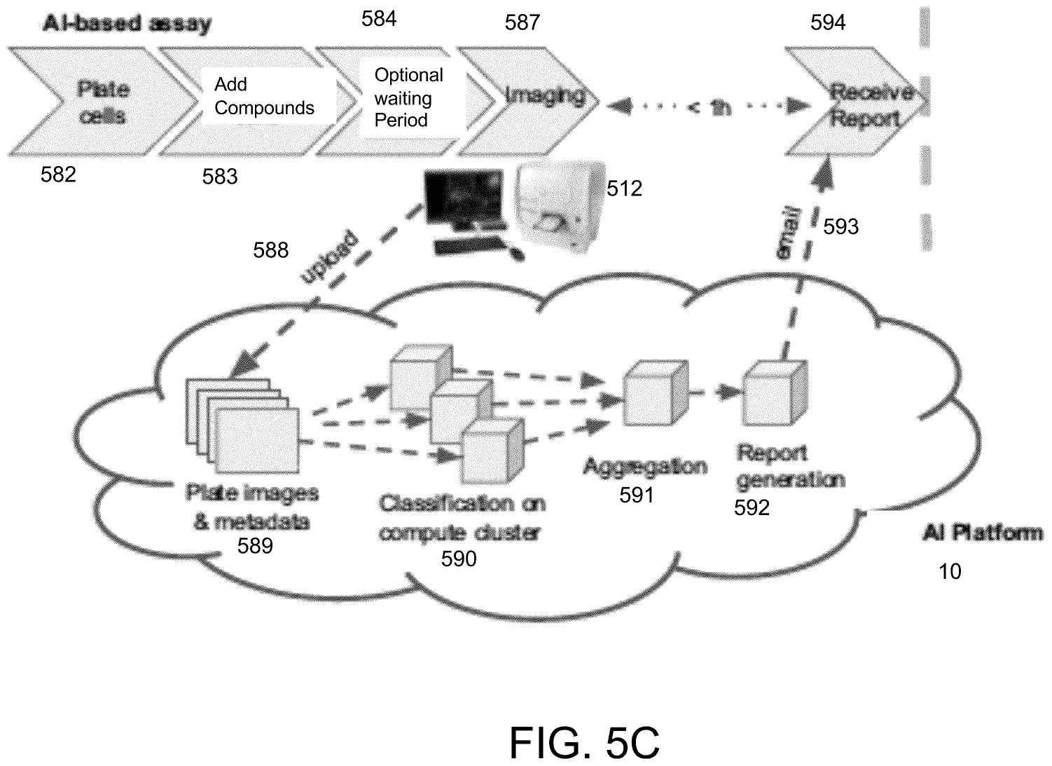

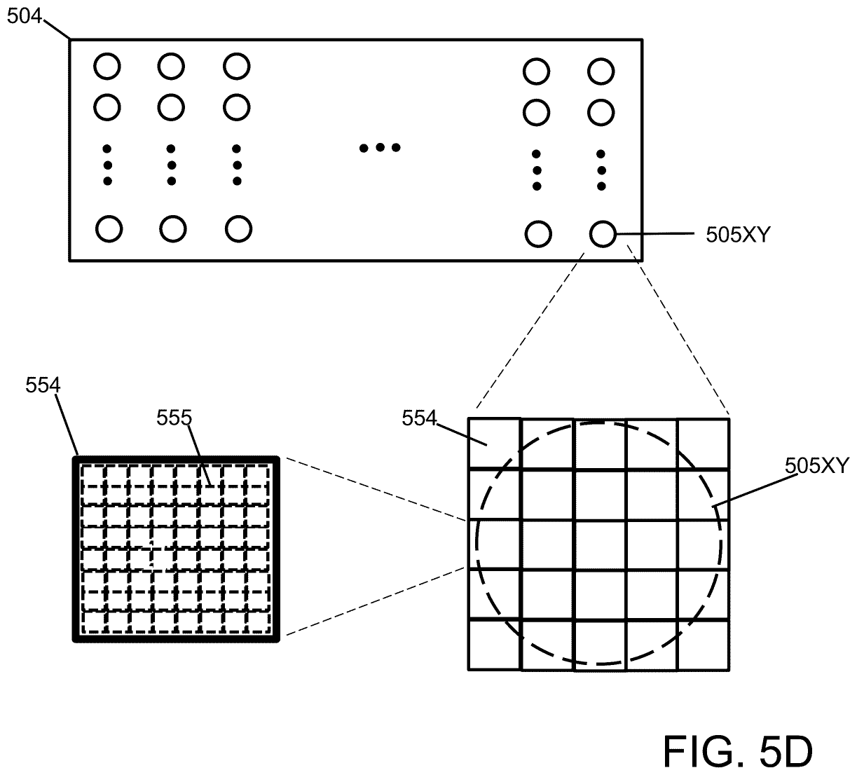

Referring now to FIG. 5A, panels 501,502503 show assay workflow of a titer plate and its training to shorten drug compound screening time. Panels 502-503 (bottom two panels) of FIG. 5A show an artificial intelligence (AI) enhanced workflow with training and classification.

Training of AI models can use images of wells of cells dosed with various drug compounds and various concentrations. A subset of wells are dosed for positive controls. Another subset of wells have no drug compound of any dosage to provide negative controls. The images of the control wells serve as the positive and negative classes for training the AI model. These images can be captured at different times after the dosing of the cells with the drug compound.

An array of a plurality of incubated wells 505XY in a titer plate 504 containing biological cells. The plurality of wells 505XY can be in a titer tray (titer plate) 504 and organized by column X and row Y. A serial dilution of drug compounds can be placed in certain wells in columns along a row. Each row can have a different drug compound. The serial dilution of drug compound in the wells can be repeated in a plurality rows to provide a replicant. After dosing the cells in the wells with the desired compounds, images of each well can be captured at one or more points in time 504A-504N as a chemical reaction is allowed to occur. High content digital images 513A-513N of each well in the tray 504 is captured by an image capture device 512, such as a microscope or automated high resolution plate imager at the desired periods of time (hours, days). A final set of images 513N can be captured of the plurality of wells 505XY by the image capture device 512. The plurality of images 513A-513N of the wells in the tray at each period can be used to train machine learning models 514A-514N. After verification, one or more of the machine learning models 514A-514N can be selected as the analysis AI models 514′ and used to classify cells in one or more wells 505XY of a titer tray (titer plate) 504 that are dosed with different concentrations of various drug compounds.

The disclosed embodiments use machine learning techniques to reduce the time screening drug compounds. In panel 502 of FIG. 5A, during a training phase, brightfield microscopy images 513A-513N can be taken at one or more time points. Images of fluorescent labels can also be captured with a different image channel of the imaging device if fluorescent dyes are used to mark cells. These brightfield images contain thousands to millions of cells that can be used to train machine learning models 514A-514N (optionally including the final assay result as training data).

Panel 503 of FIG. 5A illustrates an AI powered prediction phase. A monolayer of cells is plated into wells 505XY of the imaging plate (tray) 504 and dosed with drug compounds to form dosed cells. With a plurality of wells of dosed cells in the plate or tray, an incubation period 581A to 518B can occur. The dosed cells are incubated for a few X hours (e.g., such as between a range of four to forty-eight hours) to predict their targeted phenotype outcomes with the drug compound. During the prediction phase, the best selected model 514′ is then used to classify dosed cells at a time point (e.g., X hours) corresponding to the selected AI model 514′. The time point can be a final time 504N where there are no further changes in the dosed cells occur from the drug compounds. The result is a per-well 505XY′ prediction of the degree of effectivity of the various drug compounds to targeted phenotypes expected in the cells.

To train AI machines to discern dosed cells from non-dosed cells, high-resolution images of dosed cells captured in a brightfield (e.g., phase contrast) channel are collected. Cells in the negative controls that are non-dosed with any drug compound should not have any morphological change so they can be used for comparison.

Referring now to FIG. 5B, a block diagram of the artificial intelligence (AI) workflow (process) 550, including a training workflow (process) 551 and a classification (analytical) workflow (process) 552, for screening for phenotypes of interest in samples of biological cells under test treated with a library of compounds. After images of the biological cell samples are captured, the training workflow 551 generates a background artificial intelligence model 562 and a cell ensemble artificial intelligence model 568 that can be used by the classification workflow 552 to score the library of compounds compared to positive controls in a biological cell sample under test.

The ensemble model is a machine learning technique that combines several base AI models together in order to produce one optimal ensemble AI model. The goal is to find a single model that will best identify the desired phenotype. Rather than making one model, ensemble models take a plurality of models into account, and average those models to produce one final model.

The training and validation images 560 are preferably processed from raw image data to form rectangular image tiles 555 of M by N pixels. The M pixels by N pixels of a tile can number in the range inclusively between thirty-two pixels by thirty-two pixels and the entire pixel width and pixel height of the one or more captured images. For example, a tile can be 128 pixels by 128 pixels or 256 pixels by 256 pixels. FIG. 5D illustrates rectangular or square tiles 555 for the high-resolution digital image 554 of cells in a well 505XY. In an alternative to tiles, cells can be isolated in an image area referred to as an isolated cell image. Although tiles are preferable, the disclosed principles are applicable to both isolated cell images and tile images. The AIs can be trained to recognize phenotypes on a cell-by-cell basis or a tile-by-tile basis.

The resolution of the high resolution images that are taken of the wells can vary with the magnification of the objectives (40× or 20× magnification) of the plate imager. The resolution (tile size) can also affect the classifications provided by the AI models. The wells are tiles (tile size) Down-sampling 40× images to 20× can result in better performance for a classifier despite using one fourth the number of tiles (4 times fewer tiles) for training (and a same-size tiles in each case). Training the AIs with larger tiles (or down-sampled tiles) has the downside of losing resolution. This can be partially alleviated with overlapping tiles when processing the assay, but the ultimate resolution of the assay will be dependent on tile size.

With images obtained using a 20× objective, a tile of 256×256 pixels (256 px) is slightly larger than a typical cell, while a tile of 128×128 pixels (128 px) is smaller than individual cells. Using larger tiles can result in more accurate predictions, despite the 4-fold lower quantity of tiles in training. There are tradeoffs between tile size, resolution and accuracy that can be balanced, so embodiments can interchangeably use both 128 px tiles as well as 256 px tiles.

Blocks 561 and 562 train an AI model to prefilter background from the training and validation images 560. Because of plating limitations, some tiles can contain a background image that can decrease accuracy. To alleviate this problem, a “prefilter” network can be used to eliminate tiles that do not have cellular material. Background filter training is performed at block 561 to produce a background model AI at block 562 which can perform a background filter step at block 572 to discard tiles that have no cellular material. An advantage of the “prefilter” network and background filter step 572, it is not necessary for training and validation images 560 to be of individually isolated cells.

The other workflow path of process 551, involves training one or more AIs to detect morphological changes at the cellular level. Typically, training an artificial intelligence model that can recognize whatever you want it to recognize in images, involves exposing the AI to a dataset of images and telling the AI which subset of those images of what you want it to recognize. For example, to distinguish between dogs, cats, and stop signs, the AI would have to be told which of the images in the data set are dogs, cats, and stop signs.

The AI in the disclosed embodiments, is being trained to compare phenotypes of cells captured in images under the effects of different drug compounds and/or different concentrations of the one or more drug compounds to one or more desired phenotypes exhibited by positive controls. However, unlike in a typical AI training scenario, the phenotype differences may not be apparent to a human observer at this level of magnification and/or time after treatment. Simply put, an AI can distinguish morphological changes in a cell that would elude a human observer even when both are ‘looking” at the same images. Brightfield images, without the use of stains and dyes, contain too much information for a human observer to recognize changes. AIs are better at synthesizing images to recognize the differences. The AI training can be performed in specific ways to train the AI and its models to distinguish what a human observer cannot.

The method of training one or more artificial intelligence (AI) models to score treated cells begins by exposing the AI to a dataset of images of positive and negative controls. Positive controls exhibit target desired phenotypes. However, these desired phenotypes do not necessarily have to be directly observable a human observer. As long as an AI can be trained to differentiate the positive control from the negative control, then the phenotype is sufficiently displayed with the imaging method used. If it cannot be trained, then the effect is either not there, or is too subtle for the AI to recognize.

The use of AIs for interpreting phenotypic changes has the potential to greatly increase the use of brightfield imaging alone to analyze these screens, dramatically reducing the costs of running such screens. As with other approaches, the feasibility of doing the screen using brightfield alone and the applicability of the AI approach in general can be evaluated using the controls alone before proceeding with the entire screen.

Thus, to train the AI used in the disclosed embodiments, the training dataset consists of images of positive and negative control wells. In some embodiments, positive control wells are treated with a compound that will cause the cells to exhibit the desired phenotype. Negative control wells will not exhibit the desired phenotype. In other embodiments, the positive control can exhibit the desired phenotype due to the absence of a treatment. For example, a low-level neurotoxic effect can be induced in all cells in a screen for neuroprotective compounds, with positive controls being either absence of a neurotoxic treatment or its presence along with a neuroprotectant. In general, any phenotypic screen can be arranged in a positive sense to find compounds that induce the target phenotype, or in a negative sense to find compounds that protect cells from an undesirable phenotype.

One method of training an AI models for HCS assays, is to train level-1, individual AIs independently in parallel, with the results brought together to train a level-2 ensemble AI. The ensemble AI model 568 uses a two-layer stacking approach to training. A plurality (e.g., 12) level-1 cell AI models 565A-565N are each trained during cell class training 564A-564N using configuration files specifying starting models and their training parameters. The resultant trained cell AI models 565A-565N are used to generate predictions on the images of the biological cells to drug compounds withheld from the training images. These predictions are used during ensemble training 567 to train the level-2 classifiers, the cell ensemble model 568. The level-2 classifiers are in turn used to make predictions of on the separate validation images of the biological cells.

One or more Convoluted Neural Networks (CNNs) with a plurality of parameters settings for each can be trained on images of dosed cells at step 564A-564N. A plurality of CNN models can be trained, for a plurality of CNN AI models with a plurality of parameters, and assembled together by an ensemble model.

One method of training an AI model for drug development assays, is to train four types of AI models to drug score compounds in the screen: 1) One binary model for each positive control vs. the negative control; 2) one multi-class model on all of the positive and negative controls together; 3) one per-compound AI to evaluate dose-dependent effects for each compound independently of the controls; 4) one all-compound AI using the highest dose of each compound (or lowest effective dose) to compare all of the compound phenotypes to each other. Each image in the screen is scored by each of these 4 types of AIs.

Training sets (controls, compound data for per-compound, and all-compound AIs) are scored using n-fold cross validation (typically 5-fold). In a 5-fold cross-validation the training data is randomly split into 5 equal pools, where 4 of the pools are used to train an AI model, which is used to score the remaining pool. This is repeated round-robin 5 times so that all of the images in the 5 pools are scored. In other cases, a model trained on the control images is used to score images from each of the test compounds.

The desired morphological changes between dosed and undosed cells at the early stages of treatment can be subtle. They may not observable by a human observer. These morphological changes can differ between cell lines. Thus, an AI model trained on one cell line may not be able to differentiate between phenotype differences in a different cell line without further training.

While the AI model and the associated algorithms may not perform a screen for desired phenotypes as accurately on a different cell line, this statement should not be interpreted in the absolute. For closely related cell lines, the morphological changes may be similar enough to get an accurate screen. Although an AI model trained for a specific cell line may not accurately perform a screen on another cell line, principles of the disclosed embodiments are still applicable to all cell lines. In any case, the method of training an AI model for an HCS remains the same across all cell lines and all drug compounds. Thus, the disclosed embodiments can screen all cell lines and all drug compounds with trained AI models.

An alternative approach to machine learning involves feature-based classification using CHARM features (a large feature set used successfully in many cell-based AI applications, constituting more than 4,000 numerical image features). These features can be combined with the automated feature-classifier trainer in the final ensemble stage. The trainer can automatically select a pipeline of feature normalization, scoring, selection and classification algorithms from scikit-learn, and automatically optimizes their parameters. Several variants of this technique can be used to supplement the CNNs.

It is expected that the types of errors made by feature-based classifiers would be quite different than those made by the CNNs, thus potentially boosting the overall accuracy. We will also investigate incorporating existing AutoML packages such as Auto-sklearn and AutoKeras for automating specific level-1 and level-2 classifiers in addition to the previously mentioned fully automated feature classifiers.

Each stage of the training/classification process can be performed as an analysis module in a computer server system cloud platform, and automate the overall workflow composed of constituent software modules on a compute cluster. This can ensure that the data flow of intermediate results of the training/classification workflow are fed correctly into subsequent analyses in an automatic manner, rather than managing data flow manually.

The disclosed embodiments can also provide for storage of trained models as outputs of AI training modules and allows them to serve as inputs in subsequent screening steps. This ensures that the entire dataflow is specified as part of the workflow, which not only aids in organizing various AI models trained using various datasets and parameter sets but prevents inadvertent mistakes that are possible when these flows consist of potentially thousands of manually handled individual files.

An ensemble model can be used with different pretrained neural network architectures. A plurality of trained models can be used for each tile. Different sets may be trained for different tile sizes to find the preferred tile sizes to use. An automated feature classifier trainer tries several different feature selection and scoring techniques coupled to several different classifiers. The automated feature classifier trainer can try a range of appropriate parameters for the AI algorithms and neural networks, select the best-performing model and corresponding parameter set.

Once an ensemble AI model is trained, the ensemble AI model can be validated. The predictions of the AI models can be compared to the results of traditional assays of cells.

With the AI models (background model 562, and cell ensemble model 568) trained, they can be used in a classification process of infected biological cells captured in plate images 570. In process step 552 of the workflow, heretofore unseen plate images 570 (at least by the AIs) are classified with objects of the samples in the wells being recognized and counted. The images are analyzed and predictions are made by the AI model and the associated machine learning algorithms. The plate images 570 may not be used in a training process. The plate images 570 are prefiltered at step 572 by the background model 562 to remove background “noise”, such as any non-cellular material for example, and generate prefiltered images 574. Then, the prefiltered images 574 are analyzed by the ensemble model 568 during the cell (object) classification step 578. This can be performed in parallel on tiles of images. The analyzed images are assigned a score for each tile in the image at process step 579. During an aggregation step 580, the scores are aggregated together or averaged across all the tiles in the image and all the images in a well for a given drug compound concentration. The aggregated score can then be used to predict scores for the effectiveness of a drug compound on phenotypes at block 585.

The analysis or classification process step 552 can be used for HCS of drug compound assays. The AIs are trained and validated.

### AI Based Assay

Referring now to FIG. 5C, a workflow diagram is illustrated for an AI assisted assay. In a first step 582, biological cells are plated into all wells 505XY of a titer plate or tray. In a second step 583, the plated cells in a plurality of wells are dosed with various concentrations of a variety of drug compounds. Wells for negative controls are not dosed.

At step 584, an optional waiting period is used to allow the drug compounds to alter the biological cells. At step 587, the wells of the titer plate or tray are imaged by a high-resolution plate imager (image capture device, imaging device, digital imager, or microscope) 512. The disclosed embodiments can use an automated plate imager capable of producing brightfield images with 20× or 40× magnification. The microscope 512 can be used as well to image the whole surface of a well at high resolution (subcellular resolution) using phase-contrast with 40×-60× resolution. These types of microscopes are commonly available in imaging facilities.

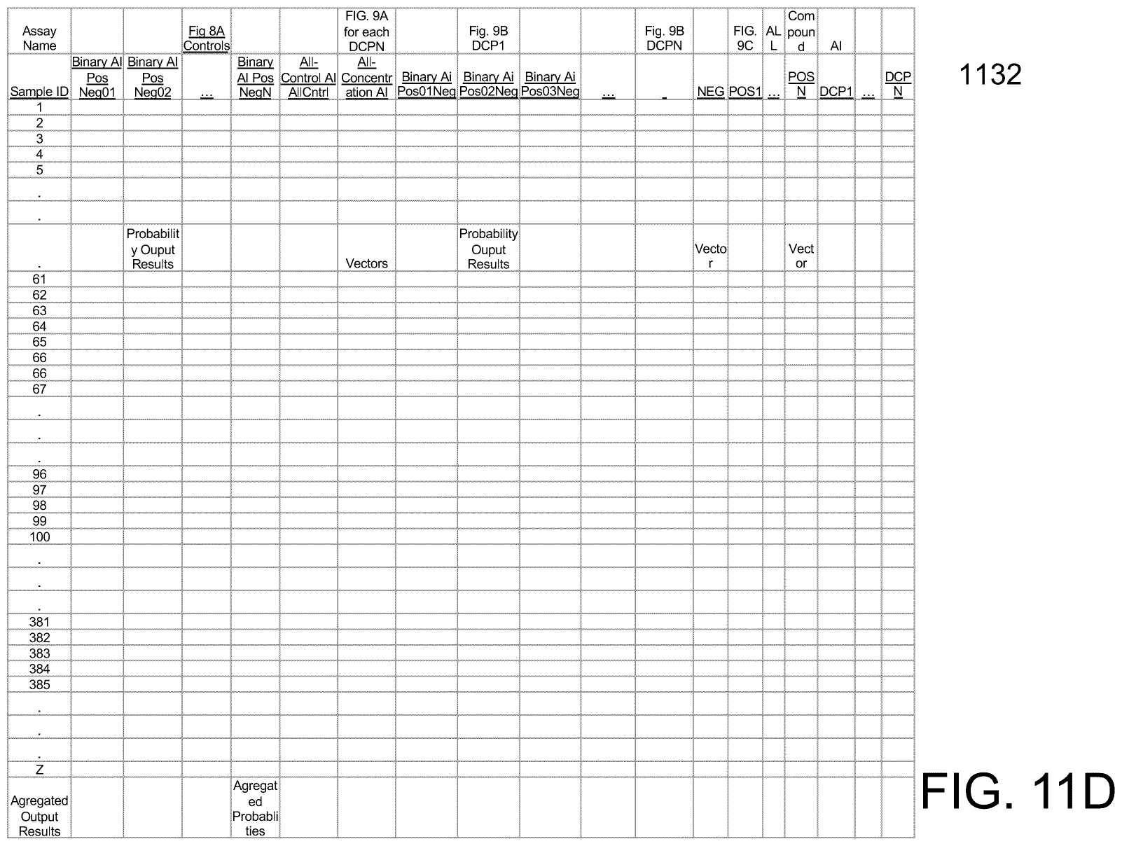

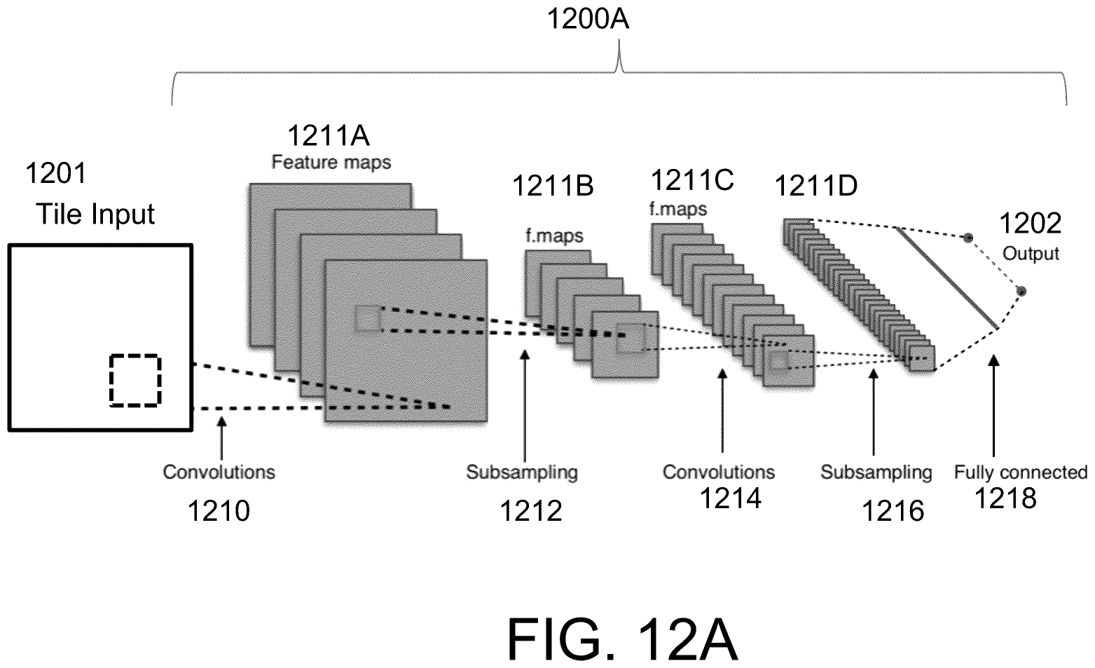

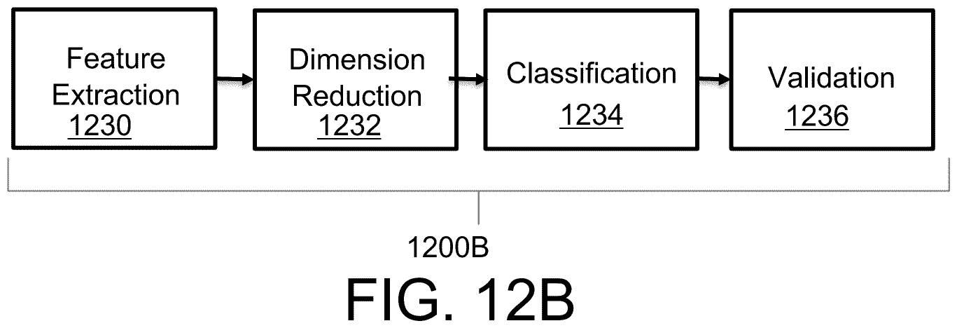

At step 588, the plate images 513A-513N and metadata are then uploaded to a cloud AI platform 10 where the images are processed. The image processing step 589 can include, down-sampling, tiling, filtering background, analysis by artificial intelligence, etc. Image processing 589 and classification steps 590 are preferably performed in the cloud-based (web-based) platform 10. However, clients with weak or no internet, security concerns, sufficient hardware, and volume of assays, can utilize client-side software to locally process their image data and run the AI assisted assays. The processed image tiles are given to the trained AI for a classification or analysis step 590. With image tiles, the classification step 590 can be performed in parallel by multiple processors in a computer cluster, such as provided by a server computer (see server computer 604 in FIG. 12). At step 591, the AI scores for the image tiles and the metadata (annotated objects) can be aggregated together. At step 592, a report regarding the drug compounds and their effectiveness in causing changes to the cells can be generated. At step 593, the report can then be sent back to a client or laboratory, such as by email. At step 594, the report can be reviewed using a user interface with a viewer window.

FIG. 5D illustrates a titer plate or tray 504 with a plurality of wells 505XY arranged in X columns and Y rows. A typical titer plate or tray may have 12 columns and 8 rows for a total of 96 wells. However, other sizes of titer plates or trays can be used, such as those having 384 or 1536 wells with additional rows and columns, that can perform more tests in parallel at the same time and generate additional images.

An imaging device can be used to capture a plurality of high resolution digital images 554 over each of the plurality of wells 505XY in the plate or tray. The digital images 554 of each well can include infected biological cells or cells exposed the various concentrations of drug compounds. In other embodiments with other imaging devices, digital images 554 of a single well, a slide, or a petri dish may be captured with infected biological cells. A digital image 554 of a portion of a well 505XY can be further partitioned into a plurality of tiles 555. Each tile 555 can be rectangular or square with dimensions of M by N pixels. The M pixels by N pixels of a tile can number in the range inclusively between thirty-two pixels by thirty-two pixels and the entire pixel width and pixel height of the one or more captured digital images 554. For example, the dimensions of a tile can be 128×128 pixels or 256×256 pixels. The tiles are preferably confluent with no gaps or overlaps.

Plate readers can be used to capture low resolution color digital images of the wells in the titer plate or tray 504 for high throughput screening (HTS) to detect a color change. In contrast to a plate reader, a high resolution plate imager is used by HCS to capture digital images with high resolution high content data to provide single cell analysis of biological samples. In drug research, the high resolution plate imager can be used to perform High-Content Screening (HCS) on the effects of different molecular compounds (drugs, pharmaceuticals) on cells of a biological sample. High resolution high content data of complex images can be quickly captured with a high resolution plate imager. Unbiased spontaneous phenotyping with intact, fixed, or live cells derived from monolayers to spheroids can be captured with a high resolution plate imager.

Typically, image-based cellular assays are used to screen a library of drug compounds for one or more effects of interest on cells (phenotypes). The compounds are applied to the cells at several concentrations (usually 4-10), typically using microtiter plates with 96, 384 or 1536 wells, with one compound concentration per well. Replicate treatments are performed (usually 2-5) in different wells to ensure the compound effect is replicable. The cells on the plates are typically imaged with an automated microscope (high resolution plate imager) that can image cells in brightfield, and/or one or more fluorescent channels, potentially using multiple focal planes to assemble 3D stacks. The resolution (magnification) employed in these screens depends on the target phenotype, some of which require subcellular resolution of less than one micron per pixel (<1 um/pixel), while others depend on visualizing multicellular structures at lower resolutions.

### Dual Machine Learning

The disclosed embodiments use supervised machine learning, where the AI models are initially trained by human involvement and then used operationally without supervision. The disclosed embodiments can train different AI systems, such as a feature-based AI and a convolutional neural nets (CNNs) AI. It has been shown that feature-based systems can work well with a relatively low number of training samples, and result in artificial intelligence models (AIs) that can discern differences to high accuracy, that are not visible to trained human observers. Similar results have been obtained with CNNs. Due to a much larger parameter space than feature-based artificial intelligence models (AIs), typically CNN artificial intelligence models require more data to train. Data can be reduced to train CNN artificial intelligence models by the use of transfer learning from an unrelated imaging problem, or the use of data augmentation. These techniques have been used successfully on imaging problems in the past, and they are now part of standard toolboxes for training CNN artificial intelligence models. The performance of the various AI models can be compared at different timepoints to determine which provides better results.

Generally, the use of three hundred example images per class has been sufficient to achieve saturation training of feature-based AI models. Generally, image data is annotated into annotated image data. The annotated image data is then collated into collated annotated image data. The collated annotated image data can be used to train artificial intelligence models (AIs). The performance of the AI models can be compared to expected results to validate one or more of the AI models. Once candidate AI models are trained and the accuracy level using the known outcomes of the training set is acceptable, the AI based assay can be validated in its final intended form to see if the accuracy is maintained.

### Platform Extensions

The web based platform was originally based on BisQue. However, web-based platform has been rewritten to make significant improvements over BisQue in many areas including: scalable viewers for data of any size, proprietary storage formats supporting millions of identified objects per image, and scalable deployments. Furthermore, several platform enhancements permit better utilization of resources and faster adoption of novel assays in order to speed the generation of results.

The web-based platform has been designed to support multiple machine learning frameworks for both comparison and future-proofing. Caffe deep learning framework is supported. Added support for both TensorFlow and PyTorch/Caffe2 is provided in order to compare the effect of the framework training on result quality. In order to train quickly on novel datasets, we utilize transfer learning based on each deep learning framework's preconfigured neural networks and datasets. Given the levelling of use that the web based permits, different frameworks can be quickly compared. Different neural network topologies can be further used as data becomes available.

Along with traditional deep-learning, a large number of pixel-based features are used to correlate early detection of viral morphology. Specialized visualization is added to demonstrate how certain features better correlate with early detection of viral damage to cells. This visualization takes the form of a heatmap of correlated sensitive features. The information gleaned from such heatmaps can be useful to researchers in understanding specifics of cell morphology. The viewer user interfaces can be enhanced to visualize the internal AI model metrics such as prediction confidence.

There are a number of other advantages to the embodiments disclosed. There is a faster turn-around time in obtaining results. The analysis of the drug compound assays on cells is more automated and can performed in higher volumes to narrow down drug compounds to undergo more comprehensive clinical drug testing.

### High-Content Screening of Assays for Drug Discovery

The initial phases of the drug discovery process can be broadly described as target discovery, lead identification, and lead optimization. Typically, lead identification requires screening through 100s of thousands of candidate compounds using a selective, highly automated, and robust assay, which can be reliably performed millions of times. These types of assays have traditionally been developed as a biochemical reaction in vitro that results in a simple, easily detectable readout like a color change, when a drug has the desired effect on its molecular target (usually a specific protein, or other cellular component). The initial stage of the discovery process was thus primarily concerned with identifying the molecular target, and establishing a cheap reliable assay for the desired effect.

In High Content Screening (HCS; or High Content Analysis, HCA), the target for the screen is a cellular phenotype of clinical interest, not necessarily a pre-identified molecular target. This redirects the target discovery stage to identifying robust repeatable cellular phenotypes rather than molecular targets. This broadens the number of potential molecular targets for the desired clinical outcome, and allows assessing drug effects directly in vivo, but with the tradeoff that the assay is no longer reducible to a color change, and instead requires acquiring and processing images of cells with a microscope to identify the phenotype of interest.

The disclosed process Auto High Content Screening (AutoHCS) changes how microscope images are analyzed in order to establish assays with reliable readouts based on target phenotypes. The disclosed process (AutoHCS) is thus useful throughout the early stages of drug development, from target discovery (or assay development), to screening, and lead optimization.

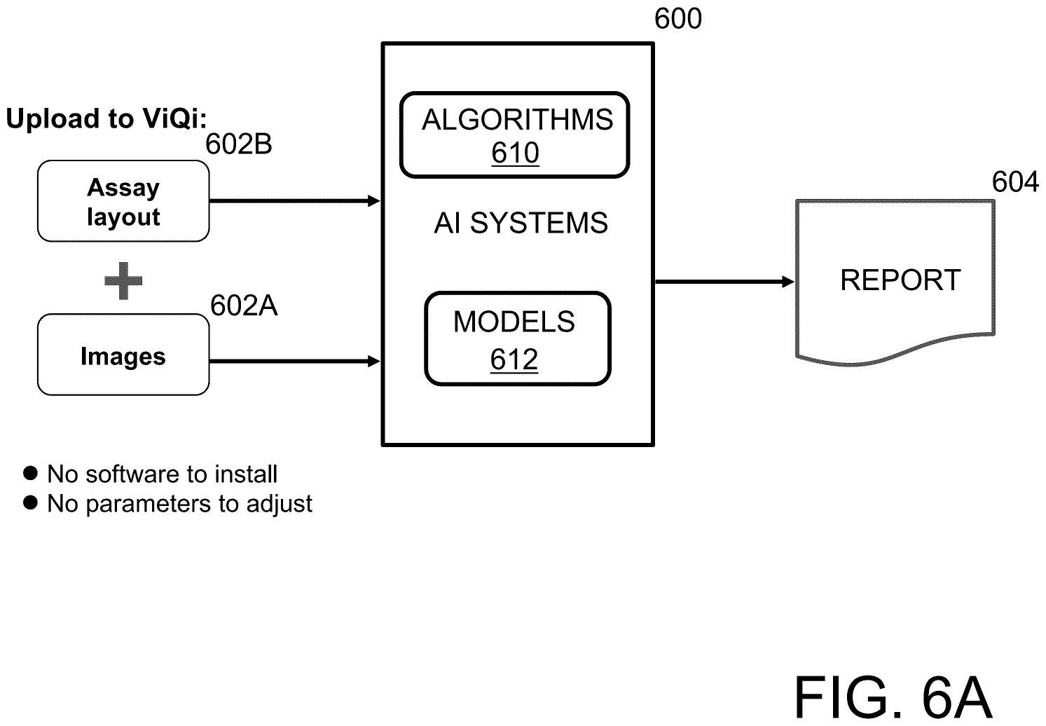

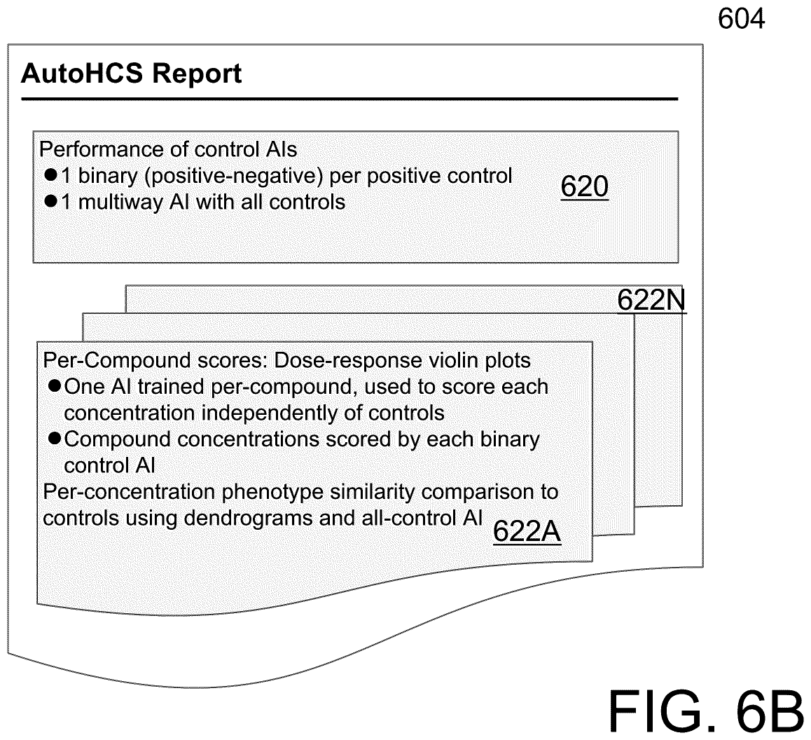

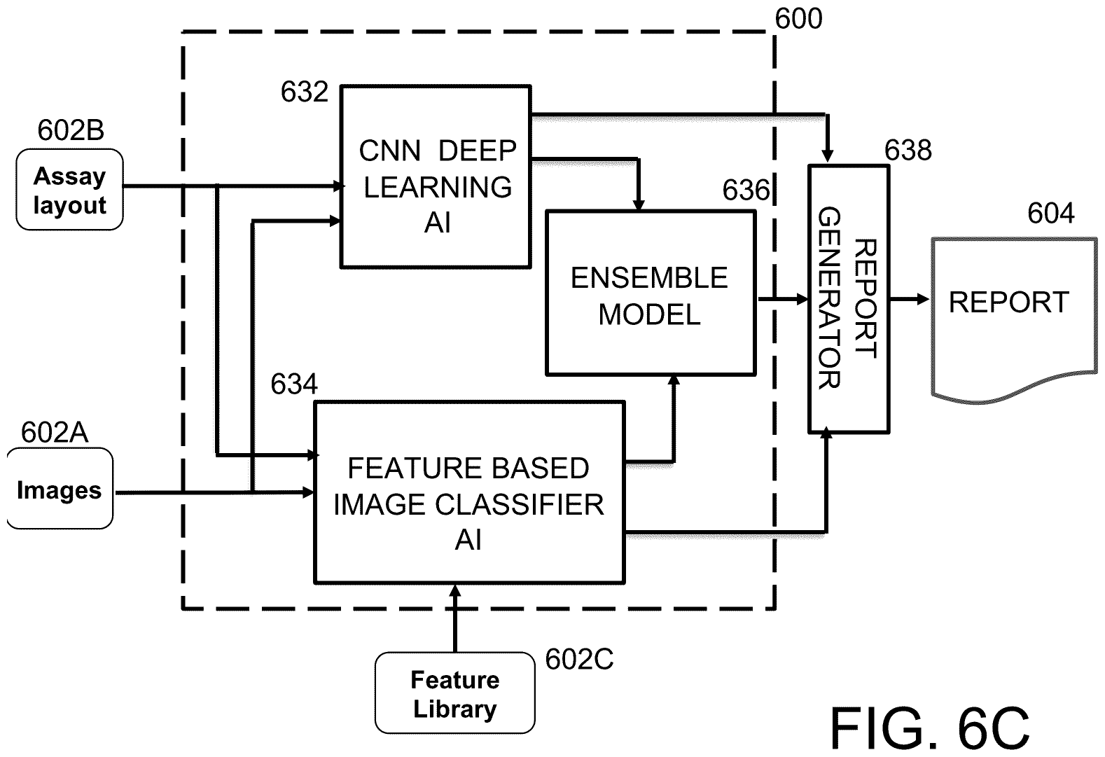

AutoHCS is a process by which image-based cellular assays for drug effects can be automated using machine learning and artificial intelligence. FIG. 6A illustrates a block diagram overview of an auto high content screening (AutoHCS or AHCS) system 600. The system 600 receives as inputs the titer images 602A of an assay from a high resolution plate imager and a plate layout 602B by means of a graphical user interface (GUI). The GUI may be used to point to a file folder or a drive to read the titer images 602A and a file that defines the plate layout 602B. The plate layout 602B can be a predefined spreadsheet file that comprises for example, the size of the titer plate, the location (and types) of different compounds, concentrations, repeats, positive and negative controls in the wells of the titer plate. The negative and positive controls are used for comparison with the new drug compounds that are being analyzed for their effects on the biological cells. Generally, the positive control sample will show an expected phenotype result with the biological cells. The negative controls are usually the biological sample without any drug compound that are not expected to change. However, a negative control can be a phenotype that is undesirable or some other condition of the biological cells. Generally, the plate layout information 602B provides some of the experimental parameters about the assay that can be used by the artificial intelligence to automate the generation of a standardized output report 604.

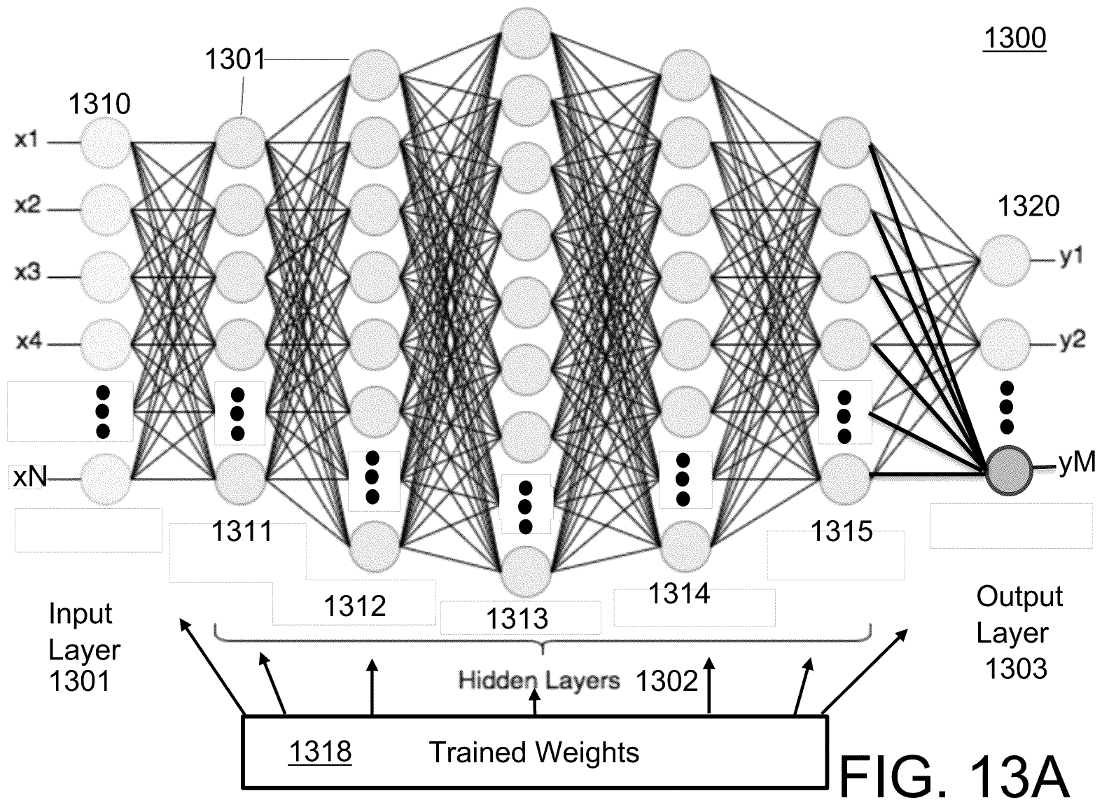

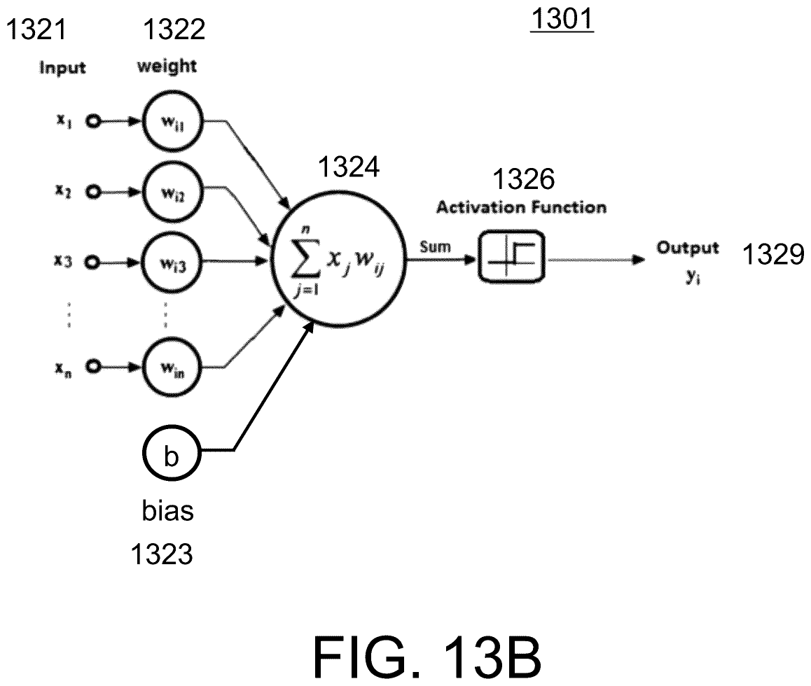

Using artificial intelligence algorithms 610 and artificial intelligence models 612, the AHCS system 600 analyzes the high content high resolution images of the wells in the plates and generates an AHCS output report 604 of how the drug (chemical) compounds effected the cells of interest. FIG. 12A illustrates a convolutional neural network system that can provide deep learning to analyze the tile input images of each well of the assay and generate the desired type output results. FIG. 13A illustrates a basic artificial neural network that can be adapted to provide certain functions in the convolutional neural network system. FIG. 13B illustrates a single artificial neuron that can be used by the neural network shown in FIG. 13A.

FIG. 6B illustrates the components of the AHCS output report 604. The AHCS output report 604 generally includes the performance 620 of control AI models and per-compound scores 622A-622N for the plurality of drug compounds. The performance scores 620 of control AI models includes one binary (positive-negative) performance for each positive control and the performance of one multiway AI model with all controls. The per-compound scores 622A-622N for the plurality of drug compounds is indicated by violin plots that shown the dose response by the cells to each drug compound. There is one AI model trained for each compound that is used to score each concentration independently of the controls. The concentrations for each compound are scored by each binary control AI. The per-concentration phenotype similarity comparisons are made by the AI model controls and an all-control AI model. The similarities and differences are shown by a similarity matrix and dendrogram plot.

Referring now to FIG. 7A, an example assay layout 700A is shown. The assay layout 700A is one of the inputs (assay layout 602B in FIG. 6C) into the AutoHCS system 600 to train the AI models 612. The assay layout may be input by a user through a graphical user interface 750A shown in FIG. 7C or as a soft copy input file 750B, such as a spreadsheet file, shown in FIG. 7D. The example assay layout 700A is shown for a 96 well titer plate. The assay layout 700A can obviously be extended to larger titer plates such as those with 384, 1536, or more wells. For example, the assay layout 700B shown in FIG. 7B has 384 titer wells associated with a titer plate that can receive chemicals, reagents, drug compounds at specified concentrations, drug solvents and/or carriers, and biological cells. It can provide three replicates and 16 rows for 16 different compounds. Each drug compound can have six different concentrations in a row of a replicant. In the 96 well titer plate layout 700A shown in FIG. 7A, eight total controls can be provided such that there can be one negative control and seven positive phenotype controls.

Referring now back to FIG. 7A, the assay layout 700A includes a number (e.g., 2, 3, r) of a plurality of replicate sections 702A-702B (replicates: Rep1, Rep2, etc.) of wells in the titer plate and a control section 720. The replicants are identically treated with the drug compounds. The control section 720 includes at least one negative control section (e.g., 1 Neg) of wells 720N; and one or more positive phenotype controls (e.g., 3 for Pos01 through Pos03) of wells 720P1-720P3 in the layout of the titer plate. The assay layout 700A further includes the numbers of a plurality of rows Y (e.g., 8) and the numbers of a plurality of columns X (e.g., 4) for each replicate 702A-702B. The plurality of rows (e.g., 1 through 8) of the replicates are typically used to test different drug compounds. The plurality of wells in the plurality of columns of each replicate are typically for increasing or decreasing dilutions of the given drug compound in the row. For the 96 well plate example, there are two replicates 702A-702B in the assay layout 700A each having four different concentrations of each drug compound in the wells of a row.

The assay layout 700A, the 96 well plate example, further includes a plurality of columns and rows of wells in the titer plate to provide controls of the overall assay. There are equal number of a plurality of wells (e.g., 8 wells) for each control (Neg, Pos01 through Pos03). In each control, each well is similarly filled to obtain multiple samples of high resolution photographs for statistical relevance. The positive controls are filled with a known phenotype of the cells that is an expected outcome from the drug compounds under test. The wells in negative control contain the same biological fluids without any drug compound in order to obtain capture multiple samples of high-resolution photographs for statistical relevance. In the 96 well plate example of the assay layout 700A, there are four controls total. There are three sets of positive control wells 720P1,720P2,720P3 for the positive (Pos01, Pos02, Pos03) controls for three different phenotypes. Each set of the one or more positive phenotype controls can target a different phenotype (e.g., a change) in the biological cells from the different compounds. With three positive phenotype controls in the layout of the 96-well titer plate, three different phenotypes of the biological cells can be targeted. In the positive control sets, there can be one positive control well for each row of a different drug compound. In the 96 well plate, each positive control set has eight control wells for the eight rows of different drug compounds.

There is one set of negative control wells 720N for the negative (Neg) control in the layout shown for the 96 well titer plate. In other titer plates with a greater number of wells, other negative control sets can be used if desired. The negative control wells 720N in the negative control have no chemical or drug compound added to the biological cells. Only inactive buffer type fluids are used for the cells in the negative control wells 720N that were also used in all other wells.

The artificial intelligence system compares each positive phenotype control with the negative control to determine if any of the drug compounds was effective to that specific targeted phenotype. If effective, one would expect to capture images of visible changes to the biological cells in the positive control wells for the positive phenotype control and no change to the biological cells in the negative control wells for the negative control.

The one or more sets of positive phenotype controls are used to generate results from one or more targeted cellular phenotypes (target phenotype) using known experimental conditions. A positive phenotype control can be used to target a known compound with a known effect, a known genetic manipulation with a known effect, or some other treatment that results in a known cellular phenotype that is of interest to be mimicked by one or more of drug compounds in a drug compound library. A positive phenotype control can itself be a known drug compound or chemical with a known effect on certain cells.

There is virtually an unlimited range of cell phenotypes that can be explored by a set of positive phenotype control wells with a high content screen. Example phenotypes include: Phenotypes exhibiting blocks in cellular division (mitosis) that can be used to find compounds that kill dividing cancer cells, which can be used for cancer chemotherapy. Phenotypes exhibiting cytotoxic effects can be screened by using apoptosis or necrosis as target phenotypes. Phenotypes exhibiting nonlethal cytotoxic effects can be used to screen for compounds affecting specialized cells, like neurons retracting their processes or hepatocytes becoming depolarized. Scratching a cellular monolayer can be used to find compounds that accelerate cell migration (phenotype) to “fix” the scratch in order to find compounds that accelerate wound healing. Scaffolds for three dimensional culture can be arranged to probe for compounds that enhance or degrade formation of 3D cultures, intercellular communication, viability, etc. of cells.

In general, any phenotypic screen can be arranged in a positive sense to find compounds that induce the target phenotype, or in a negative sense to find compounds that protect cells from an undesirable phenotype. For example, a low-level neurotoxic effect (phenotype) can be induced in all cells in a screen of a drug for neuroprotective compounds. In one embodiment, the positive controls can be the absence of a neurotoxic treatment and the negative control can be cells treated with the neurotoxic treatment. In an alternate embodiment the positive controls can be cells treated with a neurotoxic treatment along with one or more neuroprotectants while the negative control can be cells treated with the neurotoxic treatment.

For imaging, brightfield images of the wells in the title plate can be used by the AHSC system alone to probe changes in gross morphology of the cells due to the application of the drug compounds. Alternatively, brightfield images in the brightfield channel can be used with fluorescence channels by the AHSC system to probe changes in gross morphology of the cells due to the application of the drug compounds. Specific molecular biomarkers can be used in the wells of the titer plate as part of the assay to focus on specific cellular phenotypes. For example, one or more fluorescent probes can be used to mark specific phenotypes, like nuclear entry of a specific receptor. Alternatively, fluorescent probes can be used to highlight the gross morphology of cellular structures and subcellular organelles. In any case, brightfield images can be used by the AHSC system alone or with fluorescence channels to probe changes in gross morphology of the cells exposed to drug compounds.

### Graphical User Interface

Referring now to FIG. 7C, graphical user interface (GUI) 750A is shown displayed by a display device 752. The GUI 750A allows a user to set up his experiment and define the assay layout. The GUI 750A can also receive the path and/or file name for the drive that holds the high-resolution high content images that are to be input into the AHSC system and analyzed. The input field 759 can be used by the user to receive the path and/or file name for the image data.

The GUI 750A provides a number of data fields and/or pull-down menus to receive the information about the plate layout for the assays. The GUI 750A includes a plate layout panel 700 to graphically show the assay layout as the input fields are complete or selected. In the GUI 750A, a titer plate size input field/menu 753 can receive the size information about the size of the titer plate that was used in the assay. A replicant number input field/menu 754 can receive the number of replicants of rows of the drug compounds that are being analyzed for a given titer plate size. A drug compound number input field/menu 755 can receive the number of drug compounds (e.g., the number of rows) that are being analyzed in a replicate for a given titer plate size. A concentration number input field/menu 756 can receive the number of different concentrations along each row for the drug compounds (e.g., the number of columns) that are being analyzed in a replicate for a given titer plate size. An images number per well input field/menu 757 can receive the number of images that are captured for each well for a given titer plate size. A number of channels in the image data input field/menu 758 can receive the number of different channels used to capture the image data of each well in a given titer plate size. For example, the number of channels may be two, including one bright field channel and one fluorescent channel. This number of channels in the image data is related to the type of plate imager that is used to capture the images of the wells in the titer plate.

AI controls can also be specified using the GUI 750A. A positive control number input field/menu 761 can receive the number of positive controls being used for a given titer plate size. A negative control number input field/menu 762 can receive the number of negative controls being used for the given titer plate size.

While FIG. 7C illustrates a GUI 750A displayed by a display device or monitor 752, the information of the assay layout that is input could also be input into a file that is read into the ACHS system with its filename and file location. FIG. 7D illustrates a conceptual diagram of an input file with an assay layout that can be read into the AHCS system shown in FIG. 6A.

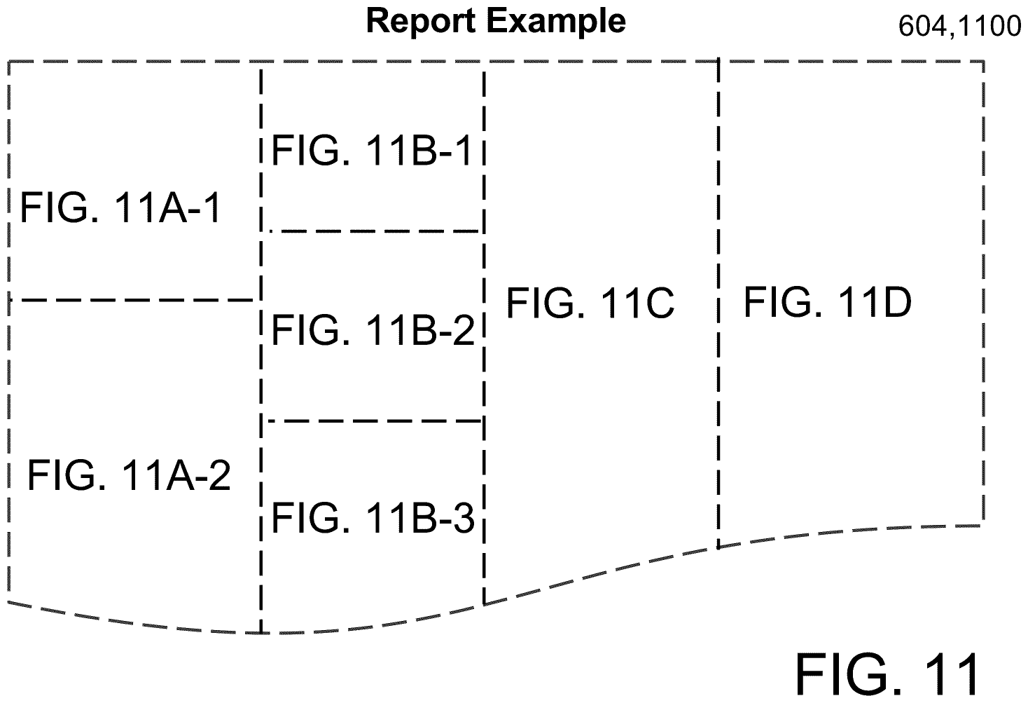

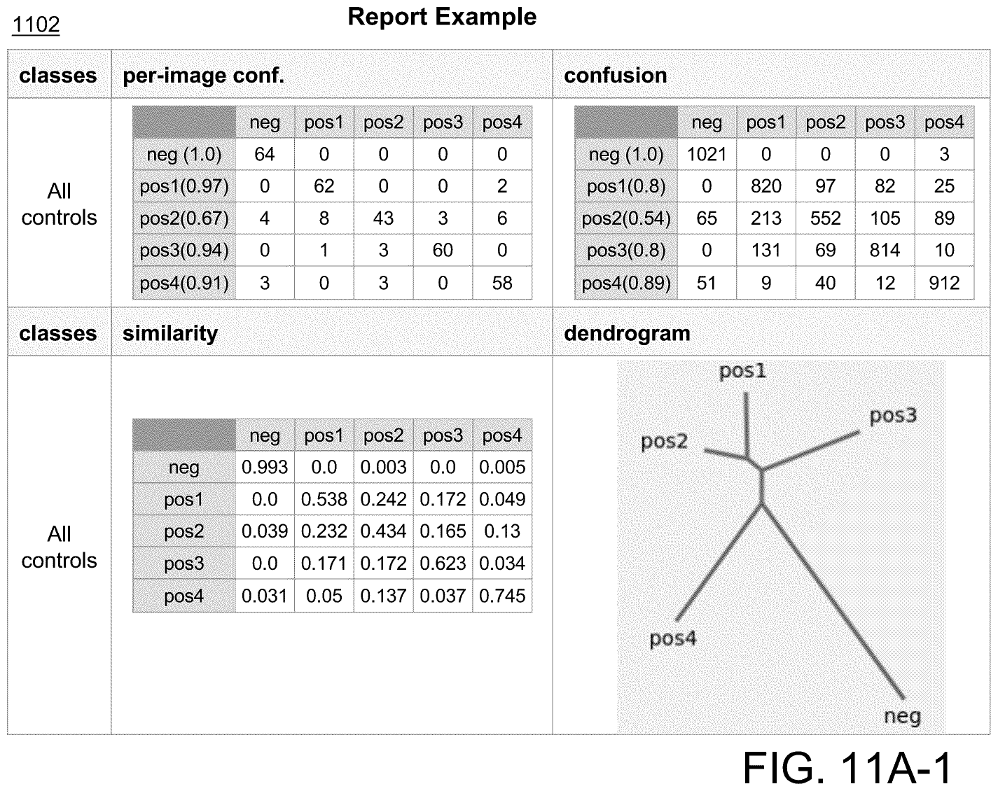

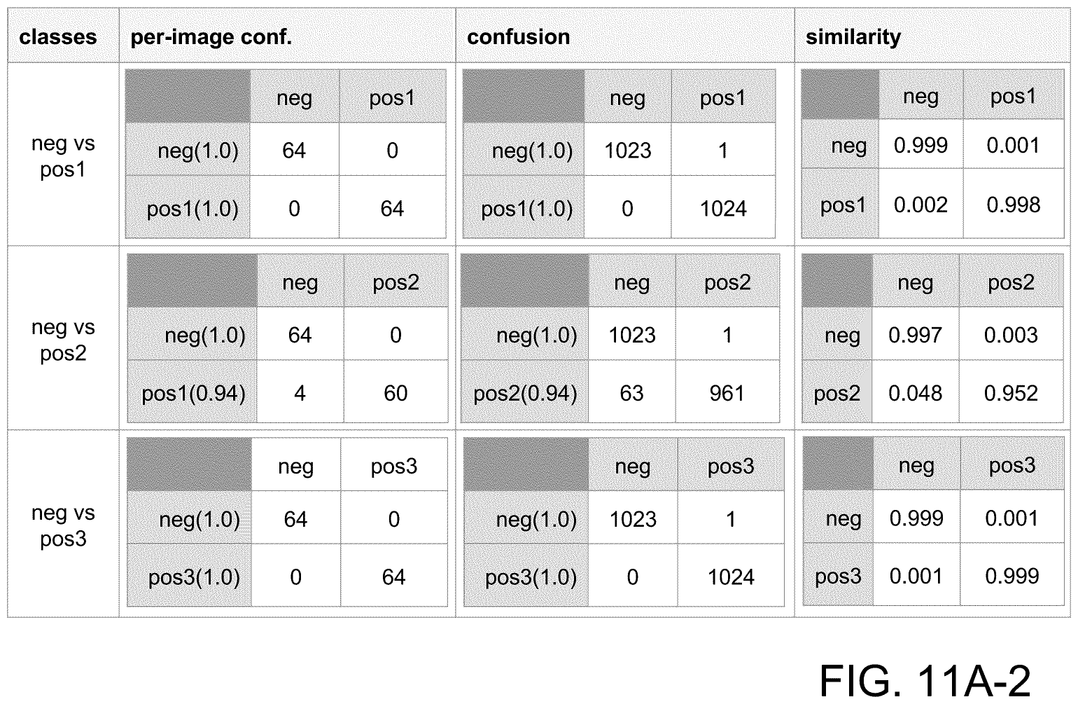

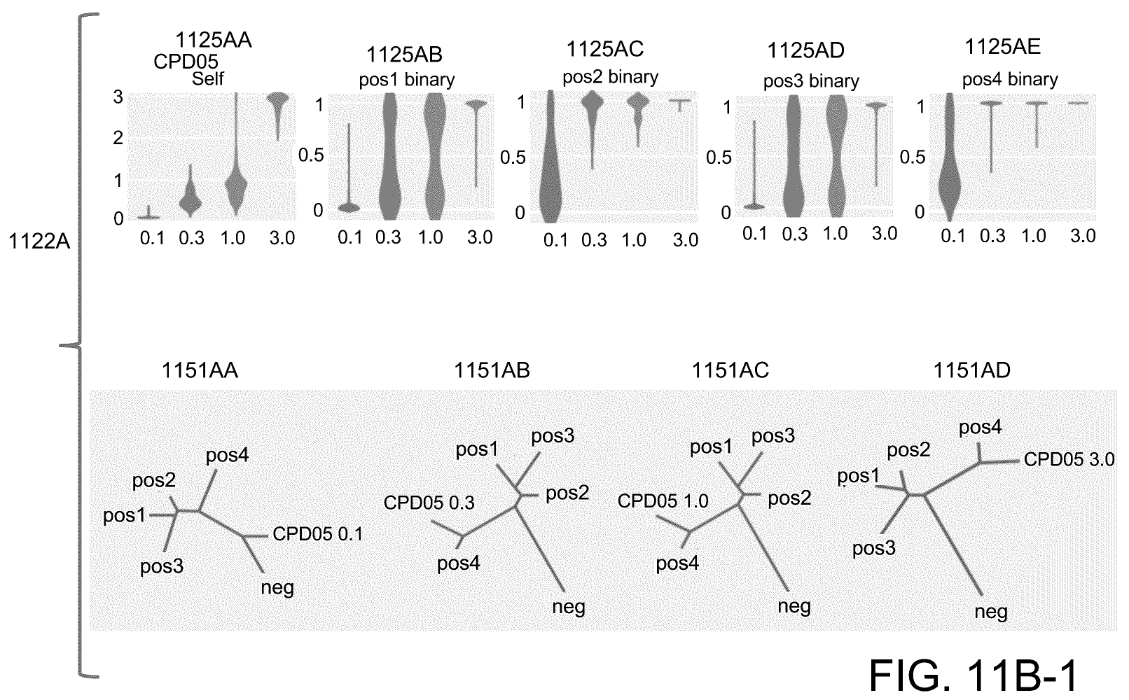

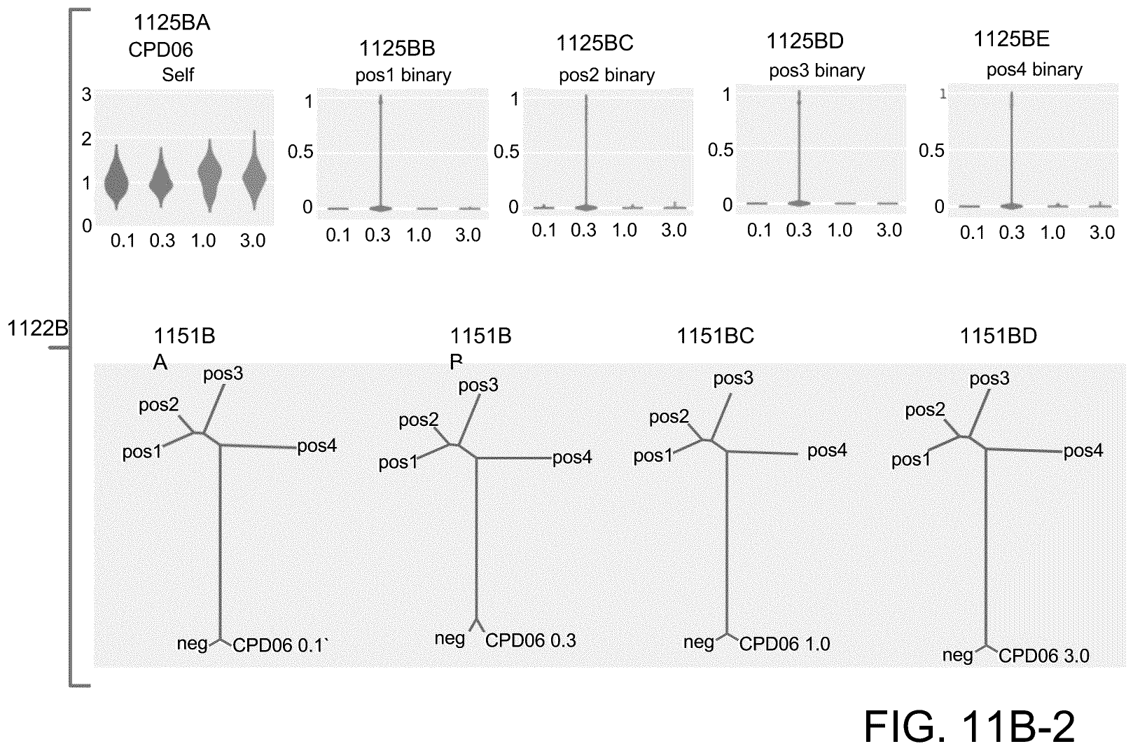

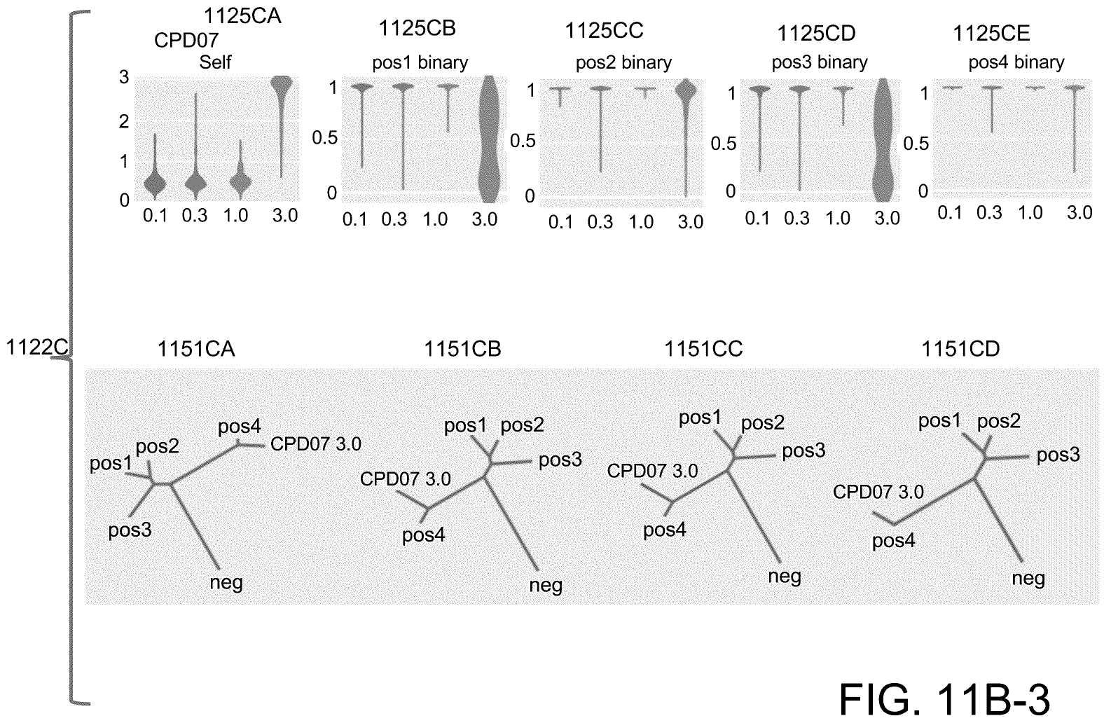

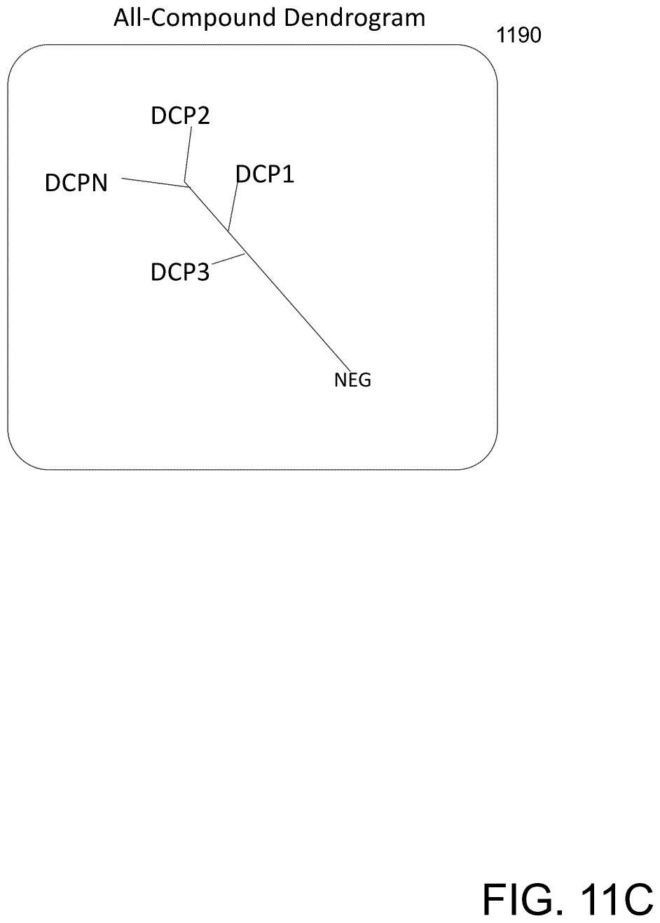

FIG. 7E illustrates a conceptual diagram of a spreadsheet input 732. The spreadsheet input 732 can be manually or automatically generated. The spreadsheet input associates the sample photographs with the assay layout for the N plates in the assay that are being analyzed. The assay name and some of the playout layout information can be shown along the first row or the last row. In the first column, the bar code of each plate of the N total plates in the assay is listed. A date and time of image capture and image name can be inserted into the next columns over. The well address, image field number of the well, and tile number associated with the image can be inserted into another set of columns. A sample photo identifying number (Sample ID) can be associated with each image name in order more readily refer to the images. The next columns Control, Replicant, Drug Compound Number, and Concentration associate the plate layout with each image. All of these inputs or at the least the Sample ID can be used in the spreadsheet raw output to associate the probability values for each image as explained herein with reference to FIG. 11D.

Referring now momentarily to FIG. 11D, a conceptual diagram of an output spreadsheet 1132 is shown. The assay name and some of the plate layout information can be shown along the first row or the last row of the output spreadsheet. In the first column, sample photo identifying number (Sample ID) associates the images with the raw data output. The sample photo identifying number (Sample ID) can also be used to associate the raw data output with the assay layout in the input spreadsheet 732. The output spreadsheet 1132 is further described to herein with respect to the output report 604.

### Advantages

The principal advantage of using machine learning for these types of AHCS screens is that the target phenotypes are used to train the AI models (AIs) by example rather than the traditional technique of describing the target phenotypes using conventional image processing algorithms. That is, the AI and its models avoids the generation of customized algorithms targeting specific phenotypes. The use of positive controls is not an imposition or a consequence of using AI models—controls have always been a necessary part of these screens. The advantage is that AI models can be trained to recognize these target phenotypes directly.

Another advantage of using AI models is that they can recognize phenotypic differences that humans cannot observe directly. It is not necessary for a human to confirm that a positive control has exhibited a phenotype by direct observation. If an AI model can be trained to differentiate the positive control from the negative control, then the phenotype is displayed with the imaging method used. If it cannot be trained, then the effect is either not there, or is too subtle for the AI model to recognize. The use of AI models for interpreting phenotypic changes has the potential to greatly increase the use of brightfield imaging alone to analyze the high content screens, dramatically reducing the costs of running such screens. As with other approaches, the feasibility of doing the screen using brightfield alone and the applicability of the AI model approach in general can be evaluated using the controls alone, before proceeding further with an entire screen or full screening of drug compounds.

### AI Models

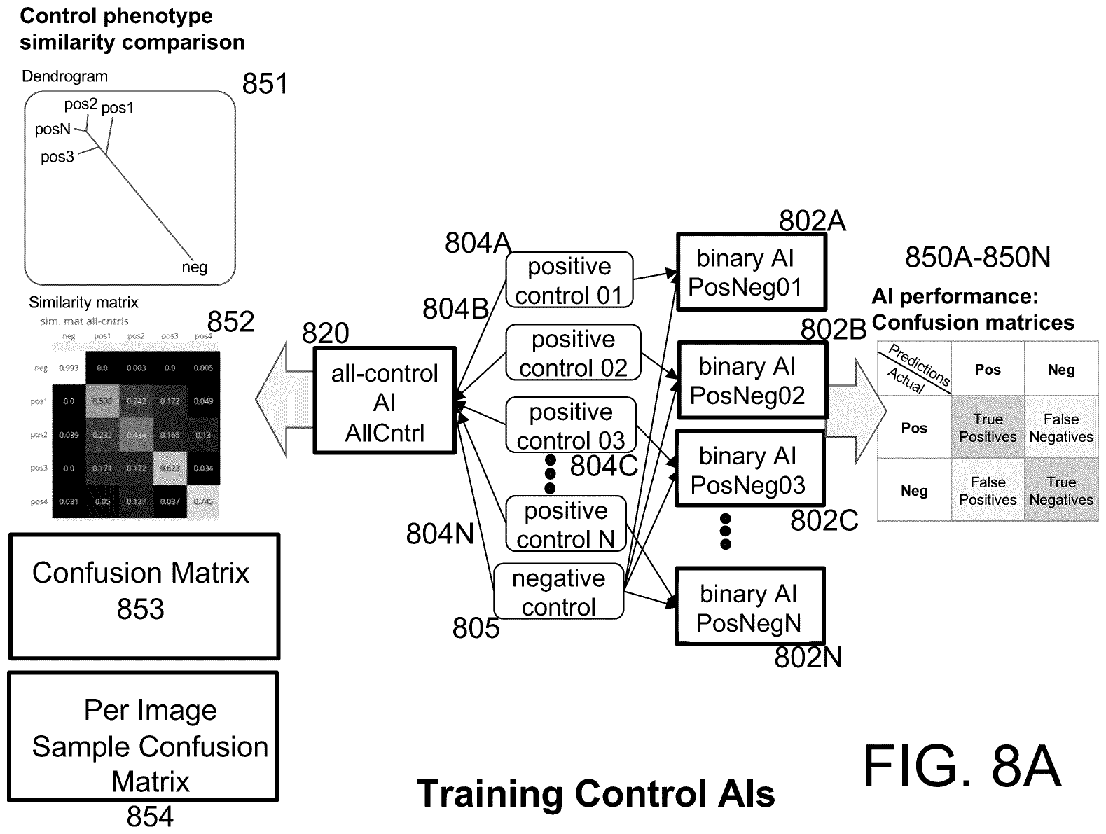

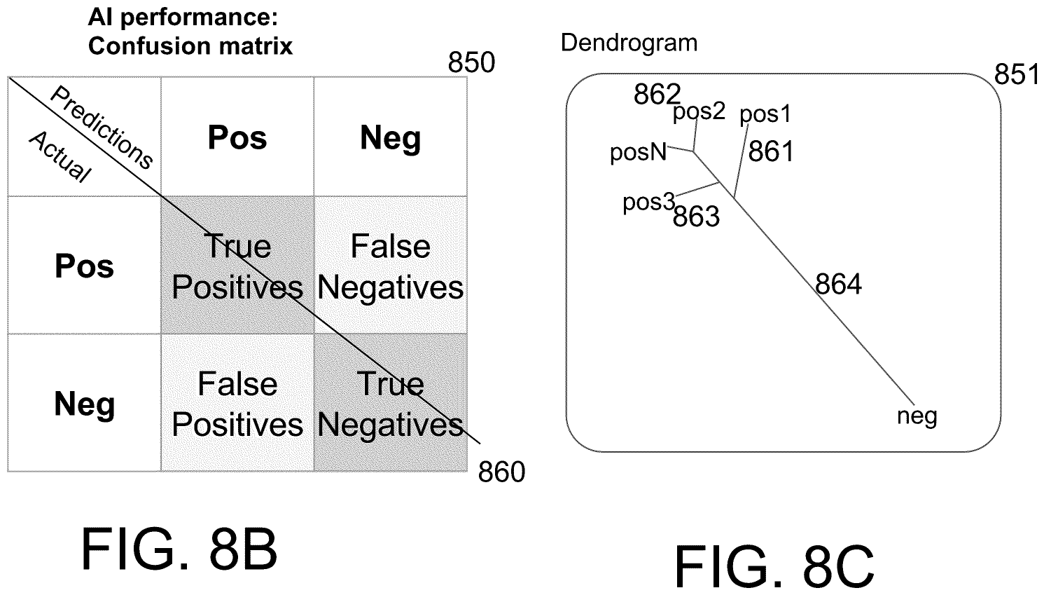

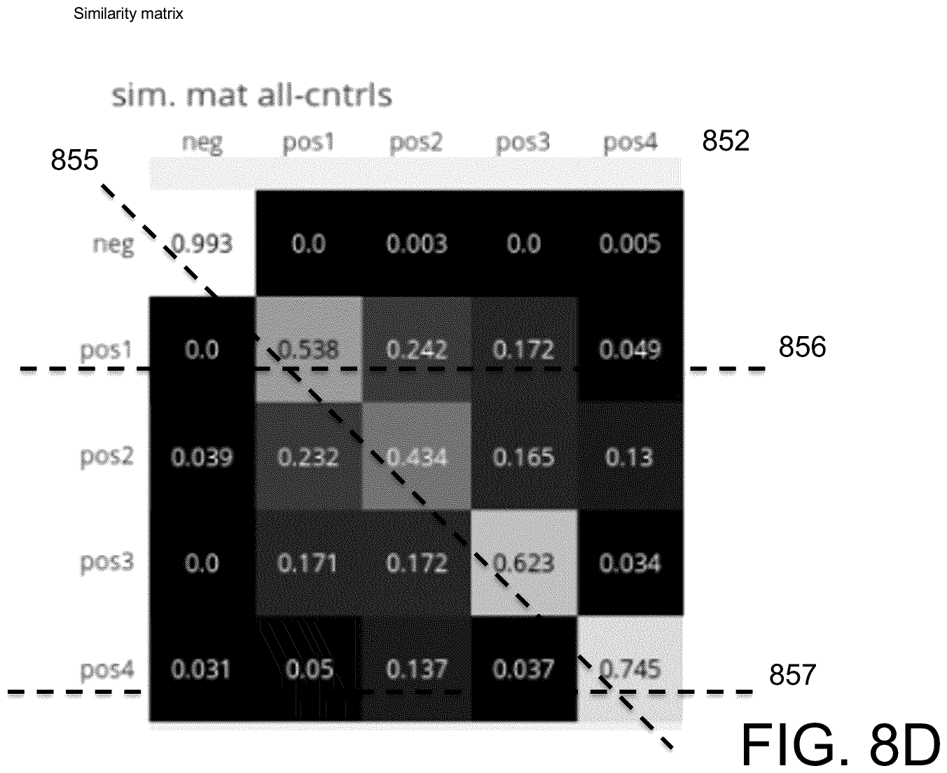

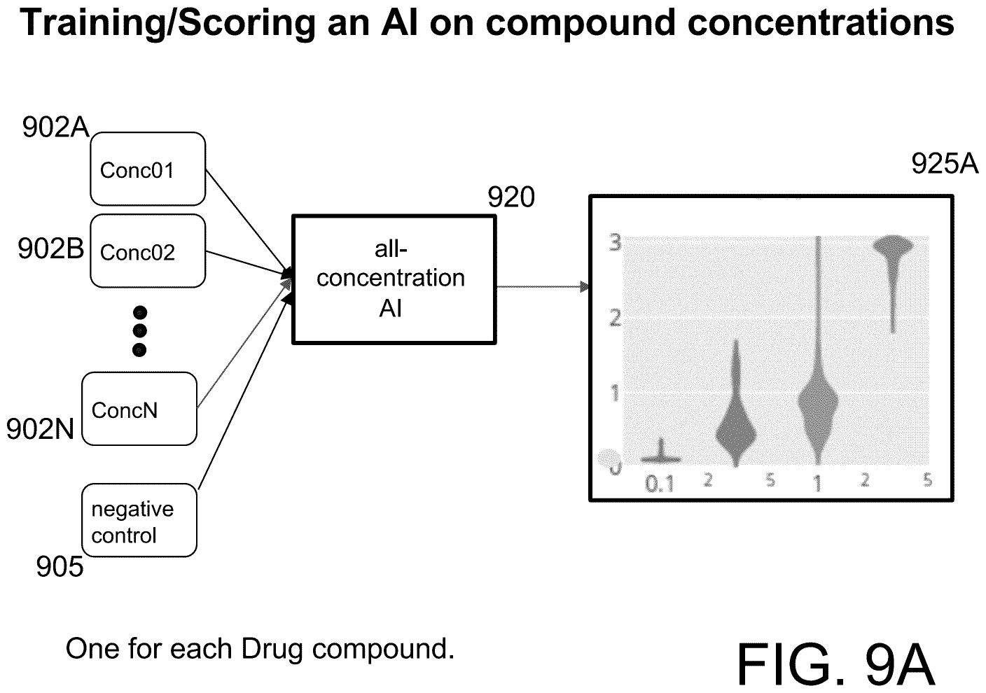

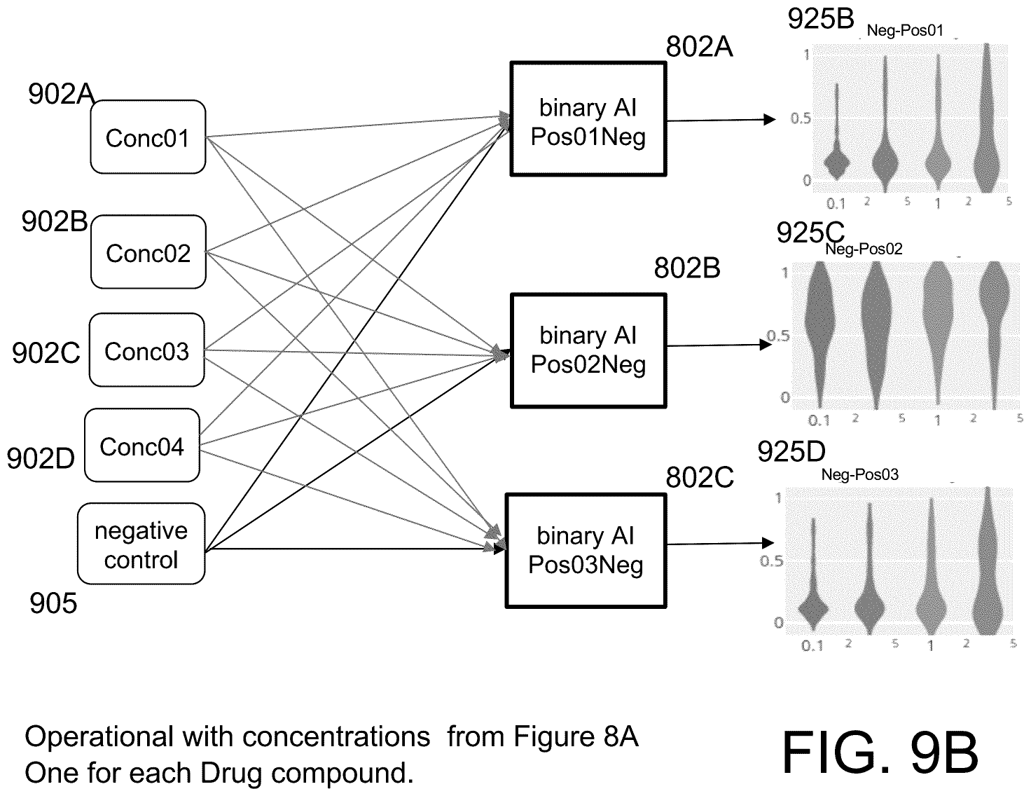

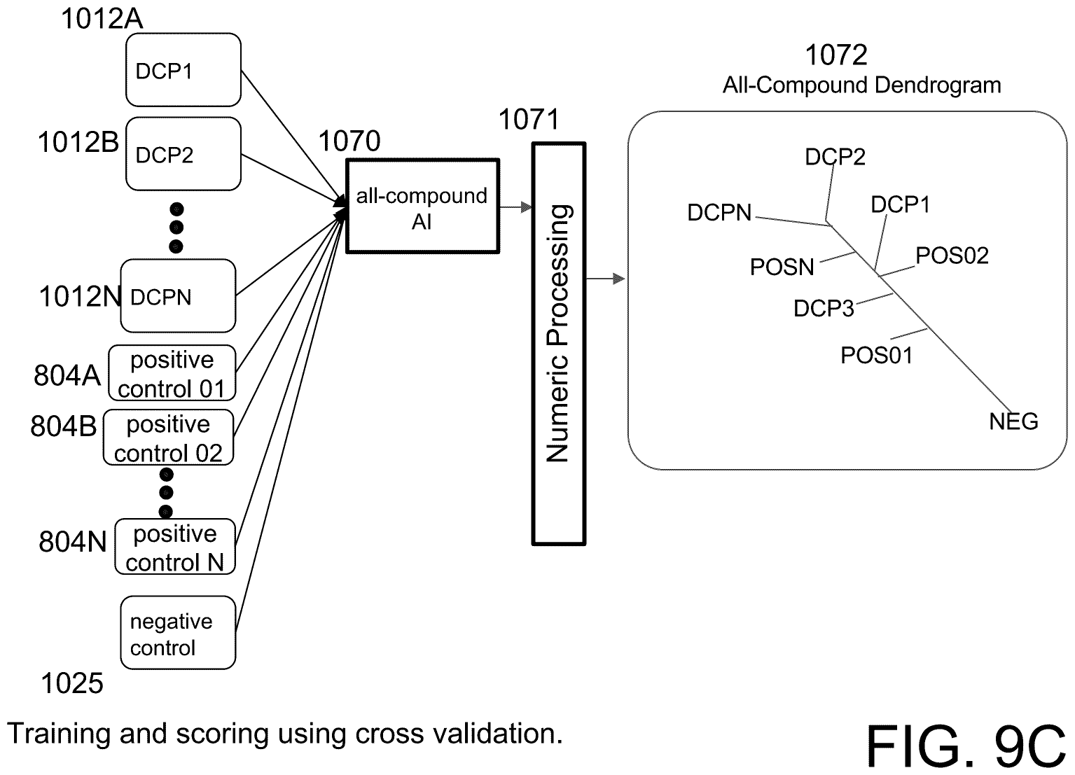

Referring now to FIGS. 8A and 9A-9C, four types of AI models 612 are trained to score drug compounds in the high content screening by the ACHS system. 1) There is one binary AI model 802A-802N for each positive control 804A-804N paired with and compared against each of the one or more negative controls 805. 2) There is one multi-class AI model 820 to combine all of the positive controls 804A-804N and the one or more negative controls 805 together. 3) There is an all-concentration AI model 920 for each drug compound with at least one negative concentration control 905 to evaluate dose-dependent effects for each compound independently of the positive and negative controls. 4) There is one all-compound concentration AI model 1070 using the highest dose of each compound (or lowest effective dose) to compare all of the compound phenotypes to each other. Each image in screening of the drug compound is scored by each of these four types of AI models.

### Training and Testing (Validation)

The AI models are trained using the scores of the controls and the outputs generated therefrom that are provided in the report 604. Training sets (controls, compound data for per-compound, and all-compound AIs) of images are scored using N-fold cross validation (typically a five-fold cross validation). In an N-fold cross-validation, the training data is randomly split into N equal pools (folds). In an initial training phase, N-1 of these pools of images are used as training data to train an AI model. The trained AI model is then scored using the remaining Nth pool of training data (sometimes referred to as testing data) for validation purposes. The training and testing (validation) of the AI model is repeated round-robin N times so that all of the images in each of the N pools are used as training data to train the AI model and at least once as testing data to score and validate the model. In other cases, an AI model can be trained on the control images from the controls and then used to score images from each of the test compounds.

Referring now to FIG. 6C, the ACHS system 600 can be flexible and utilize one or more artificial intelligence (AI) models based on AI technologies that can be selected from convolutional neural networks (CNN, or “deep learning”) imaging AI 632, and/or feature-based image classifiers 634. The feature-based image classifiers 634 uses a broad cell morphology feature library 602C (such as for example Compound Hierarchy of Algorithms Representing Morphology (CHARM) features) along with feature scoring and selection combined with modern classifiers such as support vector machines (SVM), random forest, etc. Both the deep learning AI 632 and the feature-based image classifier AI 634 can receive the assay layout 602B and the associated images 602A that are captured from the wells of the one or more titer plates that are used to run the assay. CNN-based models typically require more images to train and can be used for the control AI models (802A-802N; 820 shown in FIG. 8), and possibly the all-compound AI model 920 shown in FIG. 9.

If two or more AI models are selected (e.g., two or more CNN image AI models 632, two or more feature-based image classifiers 634, or a combination of CNN image AI models 632 and feature-based image classifiers 634), then the ACHS system includes an ensemble model 636, based on a feature-based classifier AI model or a convolutional neural network (CNN) based classifier AI model, can be used to combine results of the various AI algorithms that are used to obtain results that can be provided in the report 604. The ensemble model 636 is trained with the probability outputs from two or more upstream image classifier models (e.g., CNN deep learning image AI model 632 and/or Feature based image classifier AI model 634)) and the desired model output classes. Once trained, the ensemble model 636 can output probabilities for the classes to the report generator 638 similar to the two or more upstream image classifier models. These probabilities are aggregated and visualized in various ways by the report generator 638. The report generator 638 can perform various numeric processing, such as the numeric processing 1071 shown in FIG. 9C, to generate the visual reports such as the dendrograms, the violin plots, the confusion matrices, and the similarity matrices, as well as some of the tabular data found in the tables.

The per-compound AI models 920, one of which is shown in FIG. 9A for one drug compound, are used to detect any dose-dependent phenotype (#3 above) can use feature-based classifiers over a CNN based AI classifier. Exemplary feature-based classifiers include random forest classifiers, support vector machines, nearest neighbor classifiers, and bayes networks classifiers. For example, if there are N drug compounds with M concentrations, then there are N per-compound AI models 920 each with M concentration classes to evaluate the images of the dose-dependent phenotypes of the cells for each drug. In both cases, the disclosed ACHS software automates the training process by automatically trying a selected set of parameters. The ACHS software can either pick the best model or combine multiple models (CNN-based and/or feature-based) together into an ensemble model classifier 636 in order to generate the output report 604.

### Tile Images

The raw image input data 602A to the AI models are typically not whole images of a well, but tiles or sub-image regions of a well (with the biological cells and drug compounds therein) with a tile size chosen to maximize model performance for the control phenotypes. There is a tradeoff between tile size and sample number. Larger tile sizes present more contextual information to the AI system, typically leading to better performance, but with a fixed number of images. However, larger tile sizes also result in a smaller sample number, reducing the AI system's exposure to inter-sample variation, and making the AI system more susceptible to overtraining. The tile size is typically not a sensitive parameter. Accordingly, a range of tile sizes (e.g., two to four tile sizes) are initially tested with large steps in tile size (e.g., 2×2, 4×4, 5×5, 6×6, 8×8 tiling of a well) to choose a tile size which maximizes AI model performance for the control phenotypes. Individual tiles of an image are scored by the AI system which are then aggregated (e.g., averaged) together by an average per-image, per-well, per-replicate or per-compound concentration in the output reports for the drug compound screening.

### AI Control Models

The ACHS system uses a number of positive controls and at least one negative control in the assay and assay layout of titer plates. The positive controls are selected for detecting desired phenotypes in biological cells. In chemotherapy, for example, we would be interested in a new drug compound having a phenotype that blocks cellular division of cancer cells. If normal or cancer cells are blocked from cell division, they eventually can kill themselves. For example, five positive phenotype AI control models can be set up to investigate a variety of drug compounds to identify this desirable type of phenotype in the assays targeting specific blocks in specific cell division. The positive phenotype AI controls can investigate specific blocks of specific places and types of cell divisions, chromosome separation, and chromosome condensation due to the variety of drug compounds and concentrations. One possible negative phenotype AI control is one that identifies drug compounds that kill normal cells, not dividing, that is to be avoided. The overall score for a drug compound from these phenotype AI controls can narrow down drug compounds that don't work so well and improve the selection of those that do work well.

As another example, investigation of new drug compounds can look for those that effect neurons. Drugs that are neurotoxic or neuroprotective can be desirable. The neurons can be treated by an agent to withdraw their appendages which can be captured by high resolution high content images. You want a drug that acts as a neuroprotectant against this agent. One positive phenotype control would look for a drug compound that avoids the withdrawal of the appendages of the neuron to preserve their normal processes. Another positive phenotype control may look for drugs that are toxic to neurons to screen them out. For example, one such known compound is mercury that could be used as the positive phenotype control to identify this phenotype. Another source of positive phenotype controls could be drug compounds for a prior AHCS screen that showed the desired phenotype of the cells being tested. In any case, with the ACHS system a user can specify his/her experiment by setting up the desired phenotypes of cell to be identified in the images of the positive controls and look for those in the drug compound dosed cells in the wells of the replicates of the titer plate.

Referring now to FIG. 8A, the training of N binary AI control models 802A-802N with each respective positive control sample 804A-804N and the negative control 805 is shown to generate probability data (a measure of probability of confusion—non-recognition) for generation of respective AI performance confusion matrices 850A-850N. A first binary AI control model 802A, PosNeg01, is trained using the positive control sample POS01 804A and the negative control NEG 805 generate the first AI performance confusion matrix 850A. The Nth binary AI control model 802A, PosNegN, is trained using the positive control sample POS0N 804N and the negative control NEG 805 to generate the Nth AI performance confusion matrix 850N. Because the probability output from each binary AI is between 0 and 1, an AI performance similarity matrix can be readily generated by subtracting the probability values of confusion from the number one to obtain a probability value of similarity (a measure of probability of similarity—recognition).

A multiclass all-control AI model 820 is also trained to generate probability data for a dendrogram 851, a similarity matrix 852, a confusion matrix 853, and a per image sample confusion matrix 854. The multi-class all-control AI model 820 is trained using all the positive controls 804A-804N and the one or more negative controls 805. Numerical processing can be used on the raw data to combine all the probability data to generate a dendrogram 851 graphically representing a phenotype control comparison.

From the set of probabilities, a measure of probability is generated for each of the plurality of drug compounds, each of the one or more positive controls, and the negative control. A set of vectors is formed based on the measure of probability, for each of the plurality of drug compound, each of the one or more positive controls, and the negative control. A distance matrix for the set of vectors is calculated using the Euclidean distances (L2 norm) from each vector to every other vector in order to generate the visual representation (dendrogram).

A similarity matrix 852 can be more readily generated from the raw probability data generated by the multi-class all-control AI model 820. A confusion matrix 853, the opposite of the similarity matrix 852, can readily be generated by subtracting the values in the similarity matrix from the number one. FIG. 8D illustrates an overall control similarity matrix 852 to judge the performance of the multiclass all-control AI model 820 and the chosen positive and negative controls.

In FIG. 8A, one example confusion matrix 850, representing the confusion matrices 850A-850N, is shown for the performance of one of the N binary AI PosNegN AI models 802A-802N. The confusion matrix 850 plots positive and negative predictions along the X axis versus actual positive and negative outcomes along the Y axis. True positives and true negatives are indicated along a diagonal 860. False negatives and false positives are indicated outside the diagonal 860. A user has expectations that the positive controls are going to work with the negative control. Each of the confusion matrices 850A-850N gives the user an indication the given control works or does not. If not, the model can be further trained or different controls may be chosen by a user.

A plurality of graphical representations indicating the performance of the phenotype controls can be generated by the AI models. One graphical representation indicating phenotype controls is a dendrogram chart 851 (better seen in FIG. 8C). A dendrogram is a form of hierarchical tree diagram graphically shows clustering. The branch lengths 861-864 graphically show the level of similarity in the dendrogram chart. The AI model(s) generates probability numbers for the positive phenotype controls and the negative control(s) that undergo further numeric processing in order to generate the dendrogram chart 851. The chart 851 provides a visual comparison of the positive phenotypes with each other and the negative control. The chart 851 maps out the phenotypic relationships (phenotypic similarities) between the different drug compounds. The clustering of the plurality of positive phenotype controls (pos1 through posN) together show similarities while the different lengths of the branches 861-863 for example shows the differences between the phenotypes being examined with the positive phenotype controls. For example, cells may change shape differently in response to the different drug compounds with the differences shown by the branch lengths. As another example, the cells may change color similarly in response to the different drug compounds that can be shown by the clustering together. The negative control may not change color, for example, and be separated from the positive controls. In FIG. 8C, there is a large distance of separation by branch 864 between the negative control (NEG) and the cluster of positive controls (pos1-posN). If the positive phenotypes are appropriately selected, one would expect a good distance of separation in the branch between the negative control and the positive phenotype controls.

Referring now to FIG. 8D, another graphical representation indicating phenotype controls is a similarity matrix chart 852 that plots all the controls against each other. The numbers in each block of the matrix indicates a similarity value between zero and one for each given control versus another. The sum of the numbers in each block along a row totals to around one.

The numbers in the blocks along the diagonal 855 indicates a self-comparison of each control with itself. The probabilities of self-comparisons along the diagonal 855 in the similarity matrix should be higher than the comparisons against the negative control and the other positive controls. If not, there may be too much similarity between two controls for either one to be useful. But for the upper left corner and the lower right corner (two corners), the upper row and the left most column of the similarity matrix chart 852 show the difference between the negative control and the positive controls. The numbers in the other blocks are comparing the positive phenotypes controls against each other. If the numbers are generally greater (closer to one) in the similarity matrix, it shows more similarity between the two phenotypes. On the other hand, if the numbers are generally lower (closer to zero), it shows more dissimilarity between the two phenotypes.

Consider for example, the POS1 phenotype control for a given drug compound in row 856. Against the negative control, there is 0.0 probability of similarity between the POS1 phenotype control and the negative control in the images. Against itself, there is a 0.538 probability that the POS1 phenotype can be accurately picked out of the image. Against the second concentration and control POS2, there is a 0.242 probability of similarity with the POS1 phenotype. Against the third concentration and control POS3, there is a 0.172 probability of similarity. Against the fourth concentration and control POS4, there is a 0.049 probability of similarity. In row 856, the self-comparison of the POS1 phenotype control has the highest probability of making an accurate prediction. There is lessor probabilities of confusing the POS1 phenotype control with the other positive phenotype controls.

In row 857, the POS4 phenotype control has the 0.745, the largest similarity number in the matrix 852 indicating it is the most unique phenotype being explored with the positive phenotype controls. There is some minor similarity with the other three phenotypes as indicating by the low probability numbers 0.05, 0.137, and 0.037 for POS1, POS2, and POS3 controls respectively in row 857.

In the similarity matrix, the negative control should exhibit a lowest probability of being similar to any of the drug concentrations of the given drug compound. If not, the negative control may not be good, a positive control may not be good, or there may be something wrong with the experiments, such as possible contamination.

While the similarity matrix 852 has been discussed in detail, a confusion matrix 1112B over all controls shown in FIG. 11A-1 can be similarly generated. Furthermore, a per image confusion matrix 1112A shown in FIG. 11A-1 over all controls, for each sample image, can be similarly generated.

### AI Compound Concentration Models

Referring now to FIGS. 9A-9C, with the AI control models being trained, an All concentration AI model 920 can be trained with the concentration samples 902A-902N and the negative control 905. One All concentration AI model 920 can be trained for each of the N drug compounds DCP1-DCPN being analyzed by the assay. After training the All AI compound concentration models 920 for each drug compound, the AI control models and the all AI concentration models can be used to analyze and score each of the sample photographs of the plurality of wells in the plurality of plates in the assay. The output from the AI models and AI algorithms generate raw output data that can be numerically processed for further graphical display of plots and charts that is easier for a user to understand and make choices with regards to drug compounds and drug screening.

Referring now to FIG. 9B, for each drug compound and each phenotype, it is desirable to measure or score the effectiveness of each concentration of a given drug compound for each given phenotype of interest. To generate a score for a given phenotype and a given drug compound, the concentration samples 902A-902N for a given drug compound can be used with the binary AI control models, such as the binary AI control models 802A-802C for three phenotypes and the negative control 905. The samples of each concentration of a given drug compound are input into the binary AI control models 802A-802C to score how well they do with each phenotype. Graphs 925B-925D, generally histograms of the scores at each concentration, can be generated in order to present the scores in a useful manner to the user. The wider the band of the plot for a given concentration, the greater the number of scores exist at that value between zero and one. The narrower the band of the plot for a given concentration, there are fewer scores at that value between zero and one. The molar concentrations of the given drug compound are plotted along the X axis while an effectiveness score or change score is plotted along the Y axis.

The binary AI control models 802A-802C are looking to score between one and zero the probability that the phenotype changes in the biological cells can be found in the concentrations of a given compound. That is, across all of the different concentrations for each given drug compound, scores are generated by the AI system based on the binary AI models, and the all-compound concentration AI model 920 shown in FIG. 9A. There is one set of these AI models for each different drug compound in each row of the titer plate. Overall, the all-compound concentration AI model 920 generates a graphical representation of the concentration scores by generating a banjo or violin morphism plot (dose response chart) 925A for each compound across all phenotypes. The violin plot 925A is a kernel-density smoothed histogram of the score distributions for the image tiles for a given compound across the compound concentrations. In the violin plot 925A, the molar concentrations of the drug compound are plotted along the X axis while an effectiveness score or change score is plotted along the Y axis. During prediction, the probabilities provided by the image AI model per drug compound are used to interpolate an effective dosage of the drug compound being evaluated.

The prediction by the binary AI of each tile image of a well is a probability distribution, with one marginal probability assigned to each of the concentration classes, such that all marginal probabilities across all concentrations add up to 1.0. To get an “effective dose” score, the drug concentration with the highest marginal probability can be selected as the effective dose score. This results in discrete AI predictions corresponding to the discrete drug concentrations that are used. Alternatively, to get a continuous effective dose score for each tile image, the marginal probabilities for each dose are multiplied by their corresponding concentrations and added together.

Referring now to FIG. 9C, the drug compounds can themselves be scored against each other to see which provides the better scores for selection to undergo further drug development steps, such as clinical trials. An all-compound AI model 1070 is trained with the scores (samples) 1012A-1012N for all N drug compounds DCP1-DCPN from each All concentration AI model 920; the samples of the N positive controls 804A-804N, and the samples of the one or more negative controls 1025. After training, during operation, the all-compound AI model 1070 can be used to score the drug compounds, the positive controls representing the phenotypes, and the negative control together and generation an all-compound dendrogram 1072.

For each drug compound, image samples having the highest drug concentration are selected in one case. In another case, image samples having the most effectiveness of each drug compound are selected. Across all drug compounds, an all-compound AI model is trained based on the highest drug concentration image samples from the plurality of differing drug concentrations, all of the one or more positive phenotype controls, and the at least one negative control to generate a set of probabilities for each drug compound, for each of the one or more positive phenotype controls, and for each of the at least one negative control. A visual representation of the set of probabilities scores is generated to show how the phenotypes of the drug compounds cluster relative to each other, the one or more positive phenotype controls, and the at least one negative control.

In FIG. 9C, the new scores 1012A-1012N for all N drug compounds DCP1-DCPN, new scores of the N positive controls 804A-804N, and the new scores of one or more negative controls are all coupled into the all-compound AI model 1070. The AI algorithm and AI model 1070 generate data for vectors to plot positions on the all-compound dendrogram 1072. The raw data generated by the AI model 1070 is numerically processed by numeric processing algorithms 1071 with a processor into useful information to generate the plot in the all-compound dendrogram 1072.

For each compound and each concentration, the probability vectors are averaged to yield a centroid. Distances between the centroid and all the controls are determined to form a distance matrix. The distances are plotted of the distance matrix to form a dendrogram for each compound and each concentration to visualize at each concentration what is the most similar control to the respective compound concentration.

The all-compound dendrogram 1072 illustrates how the N drug compounds score against each other; against the positive controls POS01-POSN; and the one or more negative controls NEG. The length of the branches indicate the separation between the scores of each drug compound from each other and from the negative control NEG. In the example dendrogram shown in FIG. 9C, the drug compounds DCP2 and DCPN are shown at the top of the scoring for all the analyzed phenotypes.

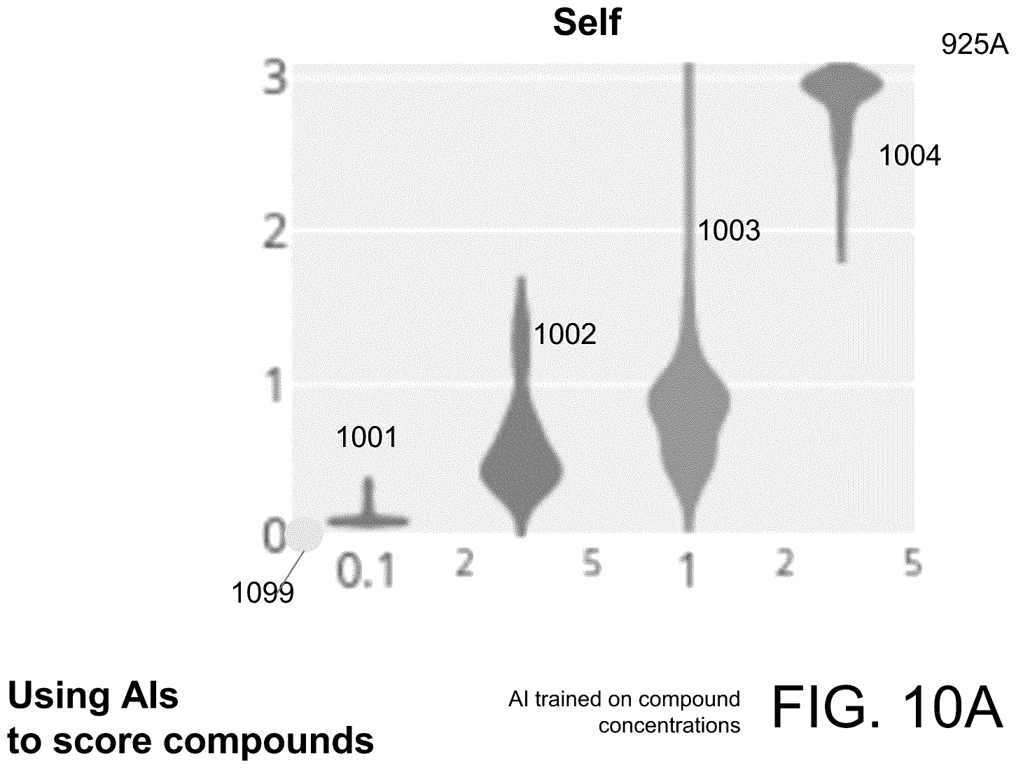

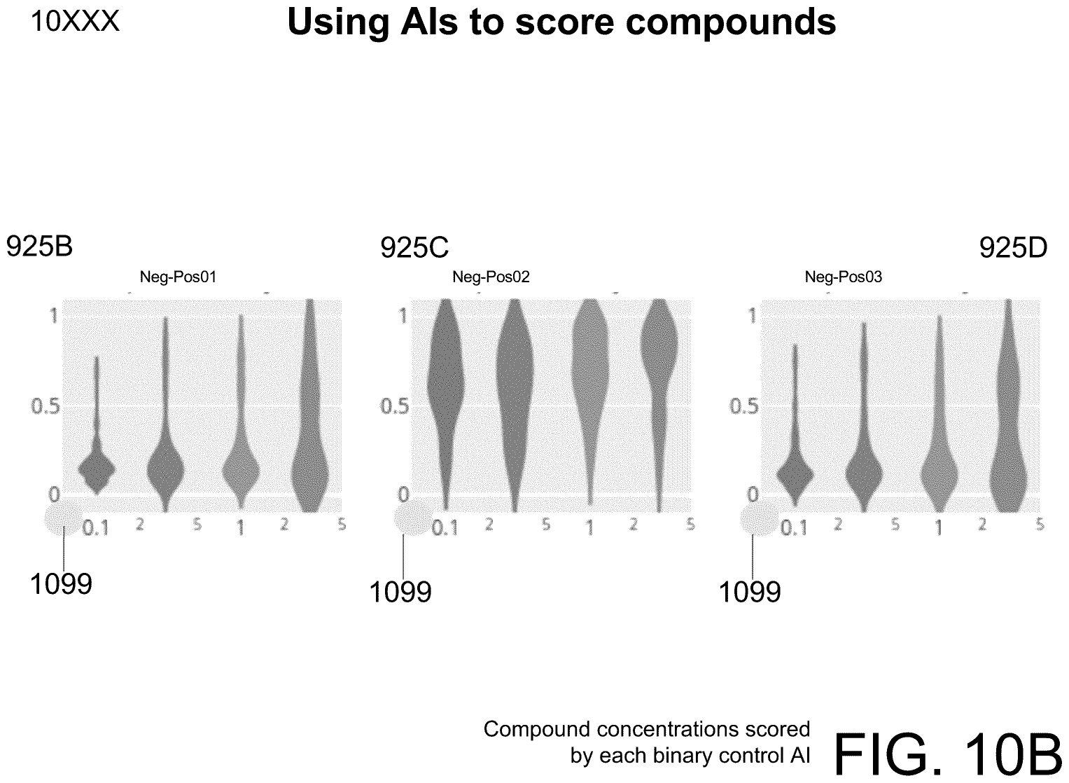

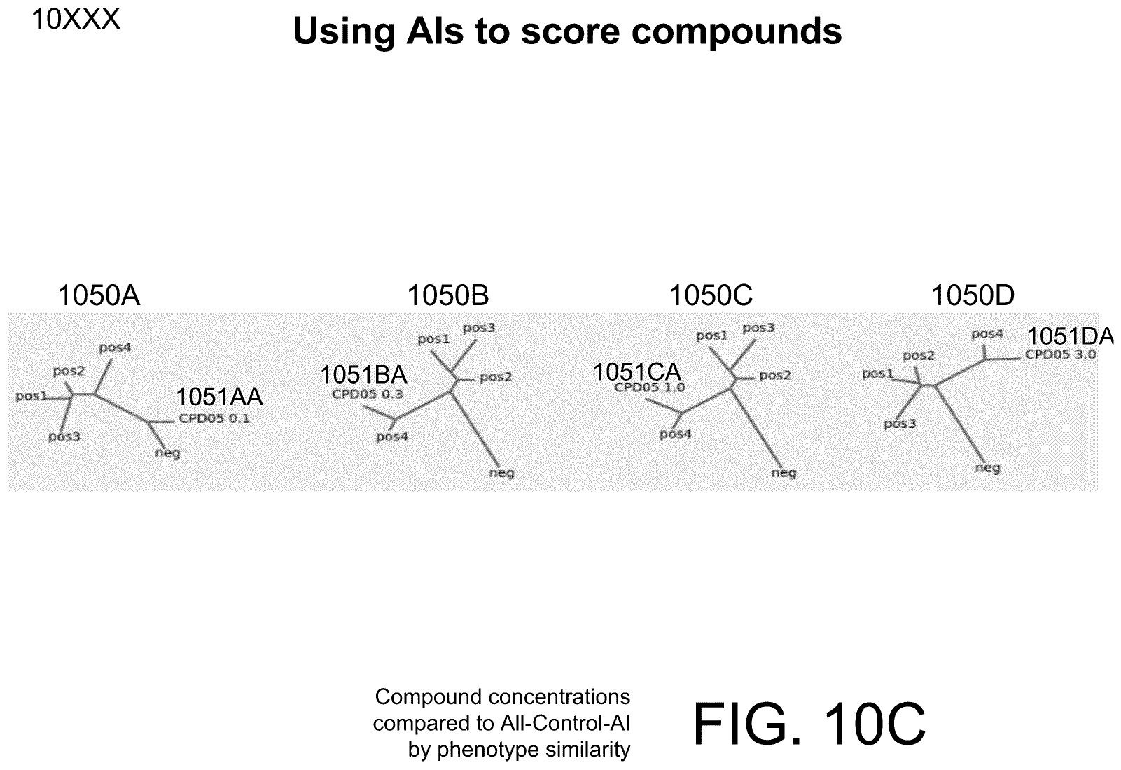

Referring now to FIG. 10A, the self-dose response chart 925A is shown that provides an effectiveness scoring of each concentration of the given compound without regards to the positive controls. In this case, the AI models are trained on the compound concentrations. The scoring indicates any kind of effect, as well as all effects, on the cells from each dose concentration. Each of dose responses 1001-1004 for the drug concentrations in the chart are generally a histogram of the different cell samples in the wells that change due to the given drug concentration. If there are N different drug compounds, there would be N sets of the self dose response charts, one for each different drug compound, in the report.

FIGS. 10B illustrates violin or banjo dose response (morphism) charts 925B-925D over four concentrations of a given drug compound for three different phenotypes and their positive phenotype controls (POS01,POS02,POS03) in comparison with the negative control. The score of the negative control is also plotted at zero concentration of the drug compound. The score of the negative control should be around zero, unless contaminated or some other effect is expected. If there are N different drug compounds for the three given phenotypes, there would be N sets of the three violin or banjo dose response (morphism) charts 925B-925D in the report.

If a drug compound has no effect whatsoever on the cells, then we expect to see a flat line of zero plotted across all the concentrations. If a drug compound has some effect on the phenotype of the cells, then we would see shifts in in the plots 1001-1004 for each concentration, such as shown by chart 925A illustrated in FIG. 10A. A score 1099 of the negative control at zero with zero concentration is shown in each of the dose response (morphism) charts 925B-925D to which a comparison is made with the positive phenotype controls at each concentration. If the negative control shows some sort of score, something likely went wrong with the assay and should be redone.

FIG. 10C illustrates dendrograms 1050A-1050D illustrating phenotype similarity for each drug compound concentration compared to the all-control (multiclass) AI model, the positive phenotype controls, and the negative control. The dendrogram charts 1050A-1050D are for the same drug compound (CPD05). Each dendrogram charts 1050A-1050D represent a different concentration of the same drug compound. For example, dendrogram chart 1050A is for the 0.1 concentration of the drug compound CPD05. Dendrogram chart 1050A is for the 0.1 concentration of the drug compound CPD05. Dendrogram chart 1050B is for the 0.3 concentration of the drug compound CPD05. Dendrogram chart 1050C is for the 1.0 concentration of the drug compound CPD05. Dendrogram chart 1050D is for the 3.0 concentration of the drug compound CPD05. If there are N different drug compounds with the four different concentrations, there would be N sets of the four dendrogram charts in the output report.

The dendrogram charts 1050A-1050D indicate where the respective concentration of the compound 1051AA, 1051BA, 1051CA, 1051CD fits amongst the plurality of controls. For example, in the dendrogram chart 1050A, the lowest concentration (0.1) of the drug compound 1051AA is plotted near the negative control by short branches. Accordingly, at this concentration the drug compound looks like the negative control, indicating that at this concentration the drug compound has little effect for any of the positive phenotype controls. In the other dendrogram charts 1050B-1050D, the drug compound 1051BA, 1051CA, 1051DA is shown clustered together with the positive controls indicating that at these concentrations the drug has some effects. In each case of the respective concentrations, the dendrogram charts 1050B-1050D show the drug compound 1051BA, 1051CA, 1051DA being nearest the fourth positive phenotype control (pos4) separated by short branches. Accordingly, at these concentrations the drug compound looks like the fourth positive control. In the dendrogram chart 1050D, the fourth and highest concentration of the drug compound, the drug compound 1051DA is shown near the fourth positive phenotype control (pos4) but above the other positive controls (POS01, POS02, POS03) and well distanced from the negative control (NEG). Accordingly, at this concentration of the drug compound in the dendrogram chart 1050D, we would expect it would look like the fourth positive phenotype control and outperform all the other positive phenotype controls.

### Example Report

FIG. 11 (FIGS. 11A-1, 11A-2, 11B-1, 11B-2, 11B-3, 11C, 11D) illustrates an example ACHS AI screening report 604,1100 for drug compounds tested in an assay of a titer plate. FIG. 11A-1 illustrates information 1102 of the performance of all the control AI models. FIGS. 11A-11A2 illustrates information for the performance of each control. FIGS. 11B-1, 11B-2, 11B-3 illustrate the concentration scores 1120 of the AI models for three different drug compounds 1122A-1122C that are being analyzed over four different concentrations for four positive phenotype controls and one negative control.

FIGS. 11A-1 and 11A-2 illustrate a report of graphical results (charts, plots, diagrams) of control AI performance scores 1102 (similar to performance scores 620) including three positive controls versus a negative control shown in FIG. 7A, and an ensemble score for all controls representing the performance of the AI models. There are three columns of matrices and one column of a dendrogram. The first column of matrices is for per-image confusion over the rows. The second column of matrices is for overall confusion over the rows. The third column of matrices is for similarity over the rows. The rows indicate various classes of controls for which the matrices are constructed. In the top or first row 1104, the class is all controls. In the second row 1105, the class is the comparison of the first positive phenotype control (pos1) compared with the negative control (neg). In the third row 1106, the class is the comparison of the second positive phenotype control (pos2) compared with the negative control (neg). In the fourth row 1107, the class is the comparison of the third positive phenotype control (pos3) compared with the negative control (neg).

In FIG. 11A-1, the top row 1104 illustrates a per image confusion matrix 1112A for all controls, a confusion matrix 1112B for all controls, a similarity matrix 1112C for all controls, and a dendrogram 1112D for all controls (neg., pos1, pos2, pos3, pos4). The top row of matrices 1112A-1112C combine the performances of the lower rows of matrices for the individual performances of different concentrations of the given drug compound. In FIG. 11A-2, the second row 1105 from the top illustrates a comparison of a positive control for a first compound concentration versus the negative control, including a per-image confusion matrix 1114A, a confusion matrix 1114B, and a similarity matrix 1114C. The third row 1106 from the top illustrates a comparison of a positive control for a second compound concentration versus the negative control, including a per-image confusion matrix 1115A, a confusion matrix 1115B, and a similarity matrix 1115C. The fourth row 1107 from the top illustrates a comparison of a positive control for a third compound concentration versus the negative control, including a per-image confusion matrix 1116A, a confusion matrix 1116B, and a similarity matrix 1116C.

In the matrices, we expect the control scores on the diagonal from the upper left corner to the lower right corner to be significantly greater than the off diagonal scores. If not, then the AI models can use more training, or alternatively, the AI neural network architecture can be designed differently to perform better.

Referring now to FIGS. 11B-1 through 11B-3, an example of compound concentration scores (collectively referred to as 1122 similar to scores 622) for three different drug compounds (CPD05, CPD06, CPD07) 1122A-1122C as part of the report is shown. The report can further include an additional five different drug compounds which can be accommodated by the 96-well titer plate shown in FIG. 7B for a total of eight rows of eight different drug compounds. With the 384-well titer plate shown in FIG. 7B, the report can further include an additional thirteen different drug compounds for a total of sixteen different drug compounds in sixteen rows. Titer plates with even more wells can be used to generate even larger reports with more compounds.

Each compound concentration score 1122A-1122C for each drug compound respectively includes a plurality of violin or banjo dosage scores 1125AA-1125AE;1125BA-1125BE;1125CA-1125CE and a plurality of dendrogram charts 1151AA-1151AD;1151BA-1151BD;1151CA-1151CD.

Referring now to FIG. 11C, the all-compound dendrogram 1190 in the report 604 can be used to select a few of the better drug compounds, out of the 100s or 1000s analyzed, that are effective for the desired phenotypes on the biological cells. Some of the compounds may cluster together around the selected phenotypes. An all-compound dendrogram 1190 provides a graphical representation of the scoring of each compound, drug compound one DCP1 through the Nth drug compound DCPN against a negative control NEG by the ACHS system. The top drug compound candidates are those that likely rise with the top scores in the dendrogram 1190, such as drug compounds DCP2 and DCPN. Instead of wading through raw data, the ACHS system uses AI to find the best scores of the analyzed compounds to select for further testing. With the output report 604, the AHCS system provides a standardized scoring system that can be used over and over again by drug companies to more efficiently investigate drug compounds.

The AutoHCS report 604,1100 can further include a table of information indicating a summary of a matching operation between file names and the plate layout for quality control of receiving and interpreting the input files (e.g., the images 602A and the assay layout 602B). The table of information indicating the summary can include a titer plate size; a number of wells and a number of images for each replicate of each compound and each concentration; a number of wells and a number of images for each dilution of drug compounds; a number of wells and a number of images for all wells containing drug compounds; a total number of the one or more differing drug compounds; a number of wells and a number of images for each positive control; and a number of wells and a number of images for each negative control.

An output device is configured to display the AutoHCS report 604,1100. The output device can be a display device (e.g., monitor 752, 1402) having a display screen to display the AutoHCS report to a user. Alternatively, the output device can be a printer (e.g., laser printer, ink jet printer, thermal printer) having a print means (e.g., laser with dry ink, ink jet head with wet ink) to printout the AutoHCS report onto paper for display to the user.

Referring now to FIG. 11D, the report 604 can further include an output results spreadsheet 1132 (likely at the end of the report) of the raw score data to provide backup to the plurality of matrices, graphs, charts, and diagrams that are generated. FIG. 11D illustrates a conceptual diagram of an output spreadsheet 1132 that would be filled in with raw scores for values, probabilities, and vectors. The last row in the spreadsheet 1132 can indicate an aggregation of the values over all the sample photos that are generated. The sample photograph identifier (Sample ID) used in the output spreadsheet 1132 is the same one assigned in the input spreadsheet 732 shown in FIG. 7E. Accordingly, the output numbers are associated with the input spreadsheet to associate the output result with the plates and the layout of the assay. While only one common column (sample ID) is shown, all the input columns of the input spreadsheet can also be included in the output spreadsheet. For reasons of simplicity, other input columns are not shown in FIG. 11D.

Besides the sample ID column, the output spreadsheet 1132 can further include columns of values for the probability output results of the Binary AI controls (FIG. 9B) for each of the N drug compounds DCP1 through DCPN. The output spreadsheet 1132 can further include columns of values (FIG. 9A) for each vector (each concentration Conc01-ConcN and NEG control) generated by the All Concentration AI model/numeric processing for each of the N drug compounds DCP1 through DCPN. The output spreadsheet 1132 can further include columns of values (FIG. 9C) generated by the All Concentration AI model/numeric processing including each vector for the negative control, each positive control, and for each of the N drug compounds DCP1 through DCPN that are used to form the dendrogram 1072.

After a first screening, a second screening can take place with the same or a subset of the drug compounds are further analyzed. The same assay with the same different drug compounds can be repeated to be sure the results are similar to those initially achieved by the ACHS system. Alternatively, for those drugs compounds narrowed down that are most effective or promising, further dilutions and/or further replicates of the selected drug compounds can undergo further ACHS screening by the ACHS system to better understand the results. Alternatively, different factors or phenotypes may be investigated after the first screening with the ACHS to investigate other facets of the drug compounds (e.g., solubility, stabilized) that may be interesting. Furthermore, the assays and chosen phenotypes can be orchestrated with the ACHS system such that that the drug compounds can be more quickly narrowed down to a chosen few in order to reduce the time to market. In any case, the drug compounds finally selected from the results of the ACHS system can then go forward in the drug development process for the more detailed clinical drug testing. With the results from the ACHS system, the number of clinical trials that are needed can be reduced and lower expenses.

The AHCS system can be used to broadly screen thousands of drug compounds down to a handful, that can go forward with the more expensive and time consuming clinical trials. Alternatively, the AHCS system can also be used to broadly detect different cell activity from a single drug compound with numerous phenotypes being selected and identified by the AI and its models.

### Artificial Intelligence Networks

Referring now to FIG. 12A, a conceptual block diagram of a convolutional neural network (CNN) 1200A is shown. The convolutional neural network 1200A can be used as the CNN deep learning AI model 632 shown in FIG. 6C. CNNs are explained by Thomas Wood article titled Convolutional Neural Network at URL www.deepai.org/machine-learning-glossary-and-terms/convolutional-neural-network. Deep learning with CNNs are further described in Chapter 7 of Deep Learning with R by Abhijit Ghatak, Copyright 2019, published by Springer Nature Singapore Pte Ltd. The CNN is generally being used to find features of the cells in the positive and negative controls and the changes from the cells by being exposed to the different concentration of drug compounds.

Generally, each tile image input 1201 from the sample photograph of the wells in the titer play undergoes a plurality of initial convolutions 1210 in a first convolution layer to generate a first set of feature maps 1211A. The initial convolutions 1210 have convolution kernels that analyze portions of the tile image to generate a plurality of initial feature images in the feature set. The initial convolutions 1210 can identify basic features in the images, such as straight edges and corners. Generally, the convolutions scale up the amount of image data to analyze in separate chunks.

The first set of feature maps then undergoes a first subsampling process 1212 to generate a second set of feature maps 1211B. The subsampling process in a subsampling layer or average pooling layer, scales down the size of the plurality of initial feature images in the feature set by averaging multiple pixels down to a single pixel. Generally, the subsampling process scales down the amount of image data to analyze in the next step.

The second set of feature maps 1211B then undergoes a second plurality of convolutions 1214 to generate a third set of feature maps 1211C. The third set of feature maps 1211C then undergoes a second subsampling process 1216 to generate a fourth set of feature maps 1211D. Additional levels of convolutions and subsampling process may be use to generate lower levels of feature maps to further simplify the analysis of the tile input 1201. With the last set of feature maps being generated, such as the fourth set of feature maps 1211D, are no longer an image but generally a one dimensional array of data with a finite length. The array of data can then be coupled into a fully connected artificial neural network 1218 that is trained to recognize certain patterns of the biological cells and then used to analyze the final feature maps and generate the desired outputs 1202.

Referring now to FIG. 12B, a conceptual block diagram of a feature-based AI system 1200B is shown. The feature-based AI system 1200B can be used as the feature-based image classifier AI model 634 shown in FIG. 6C. Feature based AI is generally discussed in CP-CHARM: SEGMENTATION-FREE IMAGE CLASSIFICATION MADE ACCESSIBLE by Uhlmann et al.; published in BMC Bioinformatics (2016) 17:51 DOI 10.1186/s12859-016-0895-y. Generally, in the feature based system, feature extraction 1230 is initially performed on sample images captured of the wells in the assay to form feature vectors. A dimension reduction process 1232 can then be performed on the feature images to reduce the dimensionality of the feature space for the feature vectors prior to classification. A classification process 1234 can be performed where a classifier is trained and used to process the reduced feature vectors of the images to determine the cellular features therein. A validation process 1236 can then be performed to validate the performance of the classifier. In the case of drug screening, the AI models can be trained and used in the classification process.

Referring now to FIG. 13A, a conceptual block diagram of a single artificial neural network system 1300 is shown. FIG. 13B illustrates a block diagram of an artificial neuron 1301 for the plurality of artificial neurons found in each layer of the neural network system 1300 shown in FIG. 13A. Each different image AI model type can be implemented with one of a plurality of artificial neural network systems 1300 with given image inputs and trained to determine the desired output results for the drug investigation and design. For example, the artificial neural network system 1300 can be trained to be used as an instance of the CNN deep learning AI model 632. As another example, the artificial neural network system 1300 can be trained to be used as the entire system 600 (less report generator) to generate results for the report 604 shown in FIG. 6C. Alternatively, a single feature-based image classifier 634 can be trained to be used as the entire system 600 (less report generator) to generate results for the report 604 shown in FIG. 6C.

A plurality of AI models of the same type can be trained and used in parallel together to provide a more accurate prediction when analyzing the tile images of the assay wells and generating desired output results from the given image inputs knowing the assay layout 602B. The results for each AI model prediction for an image tile (sub-image region) from the plurality of AI models of the same type can then be logically aggregated together by an ensemble AI model, along with the other AI model types, to form the results for the drug assay and generate the report with a report generator.

The components and functions of an artificial neural network are described by Thomas Wood in the appendix titled Convolutional Neural Network downloaded from URL www.deepai.org/machine-learning-glossary-and-terms/convolutional-neural-network, incorporated herein by reference for all intents and purposes. The components and functions of an artificial neural network are also described in Chapter 2 of Deep Learning with R by Abhijit Ghatak, Copyright 2019, published by Springer Nature Singapore Pte Ltd., incorporated herein by reference for all intents and purposes.

The artificial neural network 1300 includes an input layer 1301, one or more of a plurality of hidden neural network layers 1302, and an output layer 1303. The input layer 1301 can have at least one or more or a plurality of inputs X1-XN 1310. The output layer 1303 can have at least one or more or a plurality of outputs Y1-YM 1320. The plurality of hidden neural network layers 1302 includes one or more neural network layers 1311-1315 formed of a plurality of artificial neurons 1301. A first neural network layer 1311 of artificial neurons is coupled to the inputs 1310. A last neural network layer 1315 of artificial neurons is coupled to the outputs Y1-YM 1320. One or more neural networks layers 1312-1314 of artificial neurons can be configured between the first neural network layer 1311 and the last neural network layer 1315. Generally, the outputs of one layer of artificial neurons are coupled into inputs of each and every artificial neuron in the next layer. With fewer layers used in an artificial neural network, ordinary machine learning can be used to generate output results from the inputs.

The artificial neural network 1300 further includes a plurality of weights 1318 coupled to the plurality of computational neural networks. The plurality of weights 1318 can be trained (as trained weights) to classify objects in the sub-image tiles and to detect target phenotypes in biological cells in response to the drug compounds and concentrations in the plurality of sub-image tiles of each well in a titer plate.

FIG. 13B illustrates a block diagram of an artificial neuron 1301 for the plurality of neurons found in each layer of the neural network system shown in FIG. 13A. The neuron 1301 has a plurality of inputs x1-xN 1321, that are weighed by respective weights w1-wN 1322 and coupled into a summing function 1324. The artificial neuron 1301 further has a bias input b 1323 that is added into the summing function 1324 without any weight. Generally, the weights and the bias are the parameters of the neuron that are set by the training of the AI models.

The artificial neuron 1301 further includes an activation function 1326 that receives the summation output from the summing function 1324 and determines whether or not a value for an output yi 1329 is to be generated by the neuron or not. In some embodiments, the activation function 1326 can be a rectified linear unit (ReLU) function that outputs zero if its input is negative, and directly outputs the input value if zero or positive. In some embodiments, the activation function 1326 can be a ReLU function derivative that outputs 1 if the input value is positive, outputs zero if the input value is negative, and is an indefinite output if there is a zero input. Backpropagation during training of the weights can be an issue if zero input is reached so that the weight of the neuron never changes. In some embodiments, the parametric rectified linear unit (PReLU) function can be used as the activation function 1326. In this case, if the input to the activation function is greater than or equal to zero, then the output value is the input value. If the input to the activation unit is less than zero, then the output value is the input value multiplied by a constant number less than one, such as 0.1 for example. In some embodiments, the activation function is a logistic sigmoid function that has as its output a value between zero and 1 for all input values. In other embodiments, the activation function is a derivative of the logistic sigmoid function. While the logistic sigmoid function and its derivative are beneficial for training the artificial network because they are non-zero for all values of the input, they are more complex to compute than the rectified linear unit (ReLU) function, its derivative, and the PRELU function.

### Computer Network

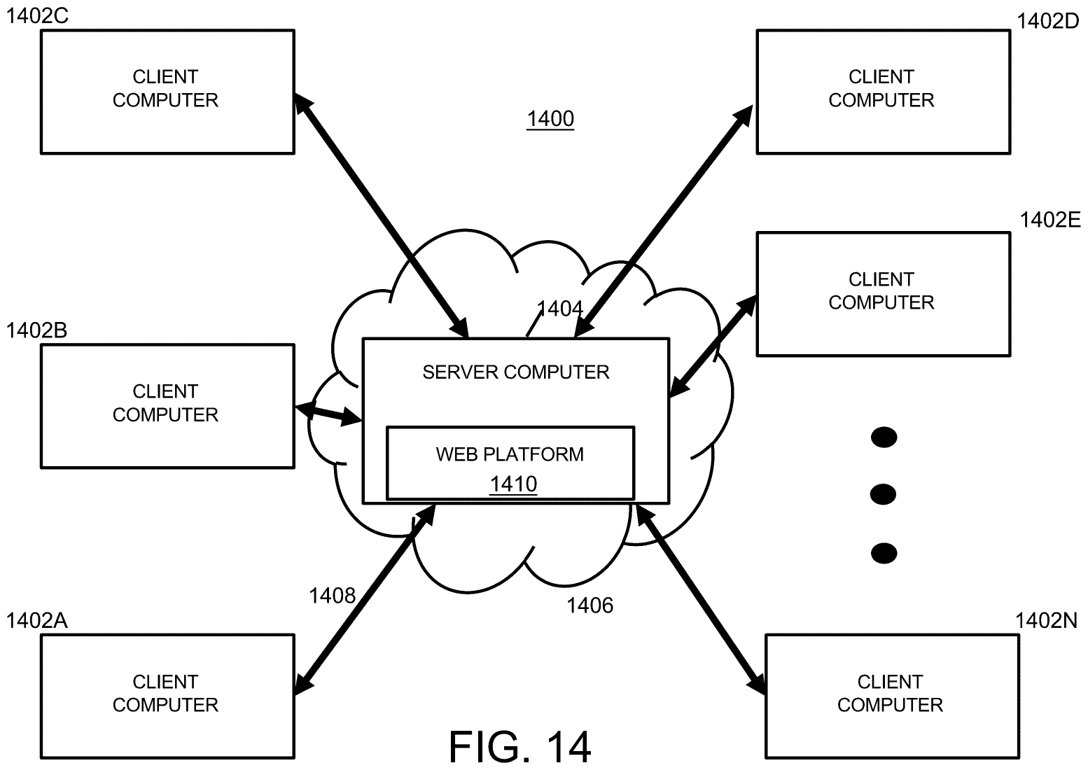

Referring now to FIG. 14, a block diagram of a client-server computer system 1400 is shown. The client-server computer system 1400 includes a plurality of client computers 1402A-1402N in communication with one or more computer servers 1404 in a server center (or the cloud) 1406 over a computer network 1408, such as a wide area network of the internet. The web-based scalable image analysis platform 1410 for AHCS drug compound screening can be executed on the one or more computer servers 1404 for access by the plurality of client computers 1402A-1402N to analyze the tile images of the wells in the titer plates of the assays. To provide the neural network nodes, the computer servers 1404 can use a plurality of graphical processing units (GPUs) that can be flexibly interconnected to process input image data and generate the desired output results established by the AI models.

### Computer System

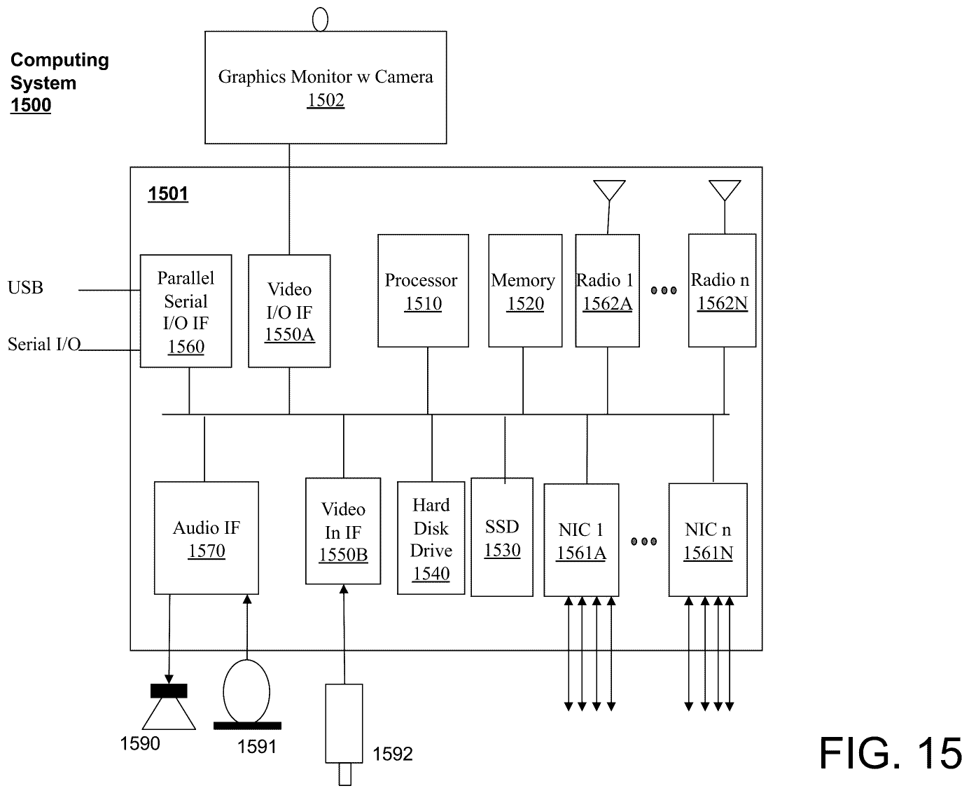

Referring now to FIG. 15, a block diagram of a computing system 1500 is shown that can execute the software instructions for the web-based scalable image analysis platform 1410 for biological cells. The computing system 1500 can be an instance of the one or more servers executing stored software instructions to perform the functional processes described herein. The computing system 1500 can also be an instance of a plurality of instances of the client computers in the wide area network executing stored software instructions to perform the functional processes described herein of a client computer to provide and display a web browser with the various window viewers described herein.

In one embodiment, the computing system 1500 can include a computer 1501 coupled in communication with a graphics monitor 1502 with or without a microphone. The computer 1501 can further be coupled to a loudspeaker 1590, a microphone 1591, and a camera 1592 in a service area with audio video devices. In accordance with one embodiment, the computer 1501 can include one or more processors 1510, memory 1520; one or more storage drives (e.g., solid state drive, hard disk drive) 1530,1540; a video input/output interface 1550A; a video input interface 1550B; a parallel/serial input/output data interface 1560; a plurality of network interfaces 1561A-1561N; a plurality of radio transmitter/receivers (transceivers) 1562A-1562N; and an audio interface 1570. The graphics monitor 1502 can be coupled in communication with the video input/output interface 1550A. The camera 1592 can be coupled in communication with the video input interface 1550B. The speaker 1590 and microphone 1591 can be coupled in communication with the audio interface 1570. The camera 1592 can be used to view one or more audio-visual devices in a service area, such as the monitor 1502. The loudspeaker 1590 can be used to communicate out to a user in the service area while the microphone 1591 can be used to receive communications from the user in the service area.

The data interface 1560 can provide wired data connections, such as one or more universal serial bus (USB) interfaces and/or one or more serial input/output interfaces (e.g., RS232). The data interface 1560 can also provide a parallel data interface. The plurality of radio transmitter/receivers (transceivers) 1562A-1562N can provide wireless data connections such as over WIFI, Bluetooth, and/or cellular. The one or more audio video devices can use the wireless data connections or the wired data connections to communicate with the computer 1501.

The computer 1501 can be an edge computer that provides for remote logins and remote virtual sessions through one or more of the plurality of network interfaces 1561A-1561N. Additionally, each of the network interfaces support one or more network connections. Network interfaces can be virtual interfaces and also be logically separated from other virtual interfaces. One or more of the plurality of network interfaces 1561A-1561N can be used to make network connections between client computers and server computers.

One or more computing systems 1500 and/or one or more computers 1501 (or computer servers) can be used to perform some or all of the processes disclosed herein. The software instructions that performs the functionality of servers and devices are stored in the storage device 1530,1540 and loaded into memory 1520 when being executed by the processor 1510.

In one embodiment, the processor 1510 executes instructions residing on a machine-readable medium, such as the hard disk drive 1530,1540, a removable medium (e.g., a compact disk 1599, a magnetic tape, etc.), or a combination of both. In a server, the video interfaces 1550A-1550B can include a plurality of graphical processing units (GPUs) that are used to execute instructions to provide the neural network nodes for the AI neural network in order to perform the functions of the disclosed embodiments. The instructions can be loaded from the machine-readable medium into the memory 1520, which can include Random Access Memory (RAM), dynamic RAM (DRAM), etc. The processor 1510,1550A-1550B can retrieve the instructions from the memory 1520 and execute the instructions to perform operations described herein.

Note that any or all of the components and the associated hardware illustrated in FIG. 15 can be used in various embodiments of a computer system 1500. It should be appreciated that other configurations of the computer system 1500 can include more or less devices than those shown in FIG. 15.

### CLOSING

Some portions of the preceding detailed description have been presented in terms of algorithms and symbolic representations of operations on data bits within a computer memory. These algorithmic descriptions and representations are the tools used by those skilled in the data processing arts to most effectively convey the substance of their work to others skilled in the art. An algorithm is here, and generally, conceived to be a self-consistent sequence of operations leading to a desired result. The operations are those have physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be kept in mind that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The embodiments are thus described. While certain exemplary embodiments have been described and shown in the accompanying drawings, it is to be understood that such embodiments are merely illustrative of and not restrictive, and that the embodiments are not limited to the specific constructions and arrangements shown and described, since various other modifications may occur to those ordinarily skilled in the art.

When implemented in software, the elements of the disclosed embodiments are essentially the code segments to perform the recited functions. The program or code segments can be stored in a processor readable medium or transmitted by a computer data signal embodied in a carrier wave over a transmission medium or communication link. The “processor readable medium” may include any medium that can store information. Examples of the processor readable medium include an electronic circuit, a semiconductor memory device, a read only memory (ROM), a flash memory, an erasable programmable read only memory (EPROM), a floppy diskette, a CD-ROM, an optical disk, a hard disk, a fiber optic medium, a radio frequency (RF) link, etc. The computer data signal may include any signal that can propagate over a transmission medium such as electronic network channels, optical fibers, air, electromagnetic, RF links, etc. The code segments may be downloaded using a computer data signal via computer networks such as the Internet, Intranet, etc. and stored in a storage device (processor readable medium).

While this specification includes many specifics, these should not be construed as limitations on the scope of the disclosure or of what may be claimed, but rather as descriptions of features specific to particular implementations of the disclosure. Certain features that are described in this specification in the context of separate implementations may also be implemented in combination in a single implementation. Conversely, various features that are described in the context of a single implementation may also be implemented in multiple implementations, separately or in sub-combination. Moreover, although features may be described above as acting in certain combinations and even initially claimed as such, one or more features from a claimed combination may in some cases be excised from the combination, and the claimed combination may be directed to a sub-combination or variations of a sub-combination. Accordingly, while embodiments have been particularly described, they should not be construed as limited by such disclosed embodiments.

1. A method for drug discovery assays using one or more artificial intelligence (AI) models, the method comprising:
receiving an assay layout defining one or more positive phenotype controls, at least one negative control, a plurality of drug compounds, a plurality of drug concentrations and their replicates in a plurality of wells of one or more plates, wherein the plurality of wells in the one or more plates to receive biological cells, drug compounds at specified concentrations, drug solvents, and/or carriers;
receiving one or more images of each of the plurality of wells in the one or more plates, wherein each image includes a plurality of tiles or one or more sub-image regions;
training one or more binary AI models based on the one or more positive phenotype controls versus negative control to generate probabilities of an input image being the positive control to which the one or more binary AI models were trained;
training an all-control AI model based on all of the one or more positive phenotype controls and the at least one negative control to generate a set of probabilities of an input image being one of the one or more positive phenotype controls or the at least one negative control; and
generating one or more visual representations of the set of probabilities to evaluate performance of the trained all-control AI model and the one or more binary AI models.

2. The method of claim 1, further comprising:
generating one or more visual representations of the set of probabilities to evaluate the phenotypes induced by the plurality of drug compounds and drug concentrations for their similarity to the control phenotypes.

3. The method of claim 1, further comprising:
for each drug compound of the plurality of differing drug compounds,
training an all-concentration AI model based on image samples from the plurality of differing drug concentrations to generate a set of probabilities for each image sample corresponding to the plurality of differing drug concentrations it was trained with; and
generating an effective concentration score for each image sample based on the set of probabilities.

4. The method of claim 3, wherein the effective concentration score is generated by
multiplying each probability output from the all-concentration AI model by each corresponding compound concentration of the plurality of differing drug concentrations to generate a plurality of products;
summing the plurality of products together to generate a plurality of effective concentration scores; and
generating a visual representation of the plurality of effective concentration scores versus the plurality of differing drug concentrations.

5. The method of claim 1, further comprising:
for each drug compound, selecting highest drug concentration image samples having the highest drug concentration;
across all drug compounds, training an all-compound AI model based on the highest drug concentration image samples from the plurality of differing drug concentrations, all of the one or more positive phenotype controls, and the at least one negative control to generate a set of probabilities for each drug compound, for each of the one or more positive phenotype controls, and for each of the at least one negative control; and
generating a visual representation of the set of probabilities scores to show how the phenotypes of the drug compounds cluster relative to each other, the one or more positive phenotype controls, and the at least one negative control.

6. The method of claim 1, further comprising:
for each drug compound, selecting most effective drug concentration image samples having the most effective drug concentration;
across all drug compounds, training an all-compound AI model based on the highest drug concentration image samples from the plurality of differing drug concentrations, all of the one or more positive phenotype controls, and the at least one negative control to generate a set of probabilities for each drug compound, for each of the one or more positive phenotype controls, and for each of the at least one negative control; and
generating a visual representation of the set of probabilities scores to show how the phenotypes of the drug compounds cluster relative to each other, the one or more positive phenotype controls, and the at least one negative control.

7. The method of claim 5 wherein,
the generating of the visual representation includes
generating, from the set of probabilities, a measure of probability for each of the plurality of drug compounds, each of the one or more positive controls, and the negative control;
forming a set of vectors based on the measure of probability, for each of the plurality of drug compound, each of the one or more positive controls, and the negative control; and
calculating a distance matrix for the set of vectors comprising the Euclidean distances (L2 norm) from each vector to every other vector.

8. The method of claim 5 wherein,
the generating of the visual representation includes
generating, from the set of probabilities, a measure of probability for each of the plurality of drug compounds, each of the one or more positive controls, and the negative control;
forming a set of vectors based on the measure of probability, for each of the plurality of drug compound, each of the one or more positive controls, and the negative control; and
calculating a distance matrix for the set of vectors comprising the Euclidean distances (L2 norm) from each vector to every other vector.

9. The method of claim 1 wherein,
the training of the AI models is supervised learning over input training images with known classifications and desired probability outputs, so that after receiving image samples, the trained model generates probabilities for each of the classes defined during training; and
the classes are negative and positive controls for the binary AI models, all of the controls for an all-control AI model; the concentrations for each compound for a self AI model; and all of the compounds at their respective active concentrations for an all-compound AI model.

10. The method of claim 1 wherein,
all sample images participate in training of the AI models and prediction with the AI models using N fold cross validation.

11. The method of claim 1 wherein,
the one or more binary AI models and the all-control AI model is based on AI technology of at least one of a group consisting of
CNN AI (deep learning); and
feature-based classifiers, such as random forest classifiers, support vector machines, nearest neighbor classifiers, and bayes networks classifiers.

12. The method of claim 1 wherein,
the training of the AI models is supervised learning over input training images with known classifications and desired probability outputs, so that after receiving image samples, the trained model generates probabilities for each of the classes defined during training; and
the classes are negative and positive controls for the binary AI models, all of the controls for an all-control AI model; the concentrations for each compound for a self AI model; and all of the compounds at their respective active concentrations for an all-compound AI model.

13. The method of claim 1 wherein,
all sample images participate in training of the AI models and prediction with the AI models using N fold cross validation.

14. A system with one or more artificial intelligence (AI) models for drug design assays using machine learning, the system comprising:
a first storage device storing one or more captured images captured at a subcellular resolution, each captured image capturing a plurality of biological cells treated with one or more known compounds over one or more concentrations;
a computer system in communication with the first storage device, the computer system including a processor and a second storage device storing instructions for execution by the processor;
a plurality of imaging artificial intelligence (AI) models stored in the second storage device for use by the processor, the plurality of imaging AI models including
one or more imaging AI models to be trained to compare each concentration of each drug compound to target phenotypes of biological cells as defined by positive controls differentiating it from a negative control,
one image AI model to be trained to distinguish all of the positive controls and the negative control from each other;
one image AI model per drug compound to be trained to distinguish the concentrations of each drug compound to detect any concentration dependent phenotype for each drug compound independently of the target phenotypes, and
one image AI model to compare all drug induced phenotypes to each other to detect phenotypic similarity between drug compounds;
wherein the plurality of imaging AI models are used with instructions executed by the processor to process the one or more captured images stored in the first storage device to generate probabilities representing a mapping between cell observations of cells captured in the images and drug compound effectiveness for each trained AI model.

15. The system of claim 30 wherein,
for the one or more imaging AI models trained to compare each concentration of each drug compound, a mapping between cell observations of cells captured in the images and the drug compound effectiveness, used during prediction, generates probabilities that are representative of the degree of similarity to the target phenotype as defined by the corresponding positive control.

16. The system of claim 15, wherein the second storage device further stores instructions for execution by the processor to
grouping the samples by drug concentration along an x axis of a chart, and
graph the probabilities for each sample into a distribution for each drug concentration along a Y axis on the chart to form a violin plot.

17. The system of claim 16 wherein,
for the one image AI model to be trained to distinguish all of the positive controls and the negative control from each other, used during prediction, the probabilities output from the AI are used as components of a probability vector to determine vector distances between vectors, wherein the vector distances represent similarity of a specific drug and concentration to a set of controls comprising the one or more positive controls and the negative control.

18. The system of claim 17, wherein the second storage device further stores instructions for execution by the processor to
for each compound and each concentration, averaging the probability vectors to yield a centroid,
determining distances between the centroid and all the controls to determine a distance matrix,
plotting the distances of the distance matrix to form a dendrogram for each compound and each concentration to visualize at each concentration what is the most similar control to the respective compound concentration.

19. The system of claim 14 wherein,
for the one image AI model per drug compound trained to distinguish the concentrations of each drug compound, used during prediction, the probabilities are used to interpolate an effective dosage of the drug compound being evaluated.

20. The system of claim 19, wherein the second storage device further stores instructions for execution by the processor to
grouping the samples by drug concentration along an x axis of a chart, and
graphing the interpolated effective dosage for each sample into a distribution along a Y axis for each drug concentration on the chart to form a violin plot.

21. The system of claim 14 wherein,
for the one image AI model to compare all drug induced phenotypes to each other, used during prediction, the probabilities for each sample represents a probability vector in a space defined by the phenotypes generated by all of the drug compounds to find similarities between phenotypes.

22. The system of claim 21 wherein,
a distance between centroids of the vectors for each compounds describes phenotype similarity between the compounds.

23. The system of claim 22, wherein the second storage device further stores instructions for execution by the processor to
for each compound at its effective concentration, averaging the probability vectors to yield the centroid,
determining distances between all-compound centroids to form a distance matrix,
plotting the distances of the distance matrix to form a dendrogram for each compound and to visualize similarities between compound induced phenotypes in the biological cells.

24-42. (canceled)
